(19) United States
(12) Patent Application Publication
(10) Pub. No.: US 2010/0150942 A1
(43) Pub. Date: Jun. 17, 2010

(54) AFFINITY PURIFIED HUMAN POLYCLONAL ANTIBODIES AND METHODS OF MAKING AND USING THEM

(76) Inventor: Thomas L. CANTOR, El Cajon, CA (US)

Correspondence Address: MORRISON & FOERSTER LLP, 12531 HIGH BLUFF DRIVE, SUITE 100, SAN DIEGO, CA, 92130-2040, US

(21) Appl. No.: 12/628,998

(22) Filed: Dec. 1, 2009

Related U.S. Application Data
(60) Provisional application No. 61/119,648, filed on Dec. 3, 2008.

(51) Int. Cl. A61K 39/40

(52) U.S. Cl. 424/163.1; 424/164.1

(57) ABSTRACT

The present invention describes a method for treating, removing or preventing a bacterial infection, which method comprises administering to a human suffering, suspected of suffering or at risk of suffering from Staphylococcus aureus (S. aureus) infection, a Streptococcus infection, Escherichia coli (E. coli) infection, Pseudomonas aeruginosa (P. aeruginosa) infection, Acinetobacter baumannii (A. baumannii) infection, Enterococcus faecium (E. faecium) infection and/or Clostridium difficile (C. difficile) infection, an effective amount of human polyclonal antibodies affinity purified from a human blood sample with an antigenic preparation comprising cellular and/or secreted antigen(s) from bacterial cells selected from S. aureus, a Streptococcus, E. coli, P. aeruginosa, A. baumannii, E. faecium, C. difficile or a combination thereof, and optionally, wherein said affinity purified human polyclonal antibodies are purified (e.g., as made more concentrated as compared to the starting or unpurified material) relative to the same human polyclonal antibodies in the unpurified or non-affinity-purified human blood sample, e.g., intravenous immunoglobulin (IVIG) sample, and/or also optionally, wherein said affinity purified human polyclonal antibodies are specific for the bacterial antigens used in the affinity purification, and/or further optionally wherein the affinity purified human polyclonal antibodies are substantially free of human antibodies that specifically bind to non-bacterial antigens in the human blood sample. Pharmaceutical compositions for treating bacterial infections, comprising an effective amount of human polyclonal antibodies affinity purified from a human blood sample with an antigenic preparation comprising cellular and/or secreted antigen(s) from S. aureus, Streptococcus, E. coli, P. aeruginosa, A. baumannii, E. faecium, C. difficile or a combination thereof, are also provided.

### CROSS-REFERENCE TO RELATED APPLICATIONS

This application claims the benefit of U.S. Ser. No. 61/119,648, filed Dec. 3, 2008, which is incorporated herein by reference in its entirety.

### FIELD OF THE INVENTION

This invention generally relates to the field of bacterial infections, particularly to immunological compositions and therapeutic uses thereof, i.e., methods for treating and preventing bacterial infections, and more specifically to the use of affinity purified human polyclonal antibodies for the prevention, removal, treatment and/or monitoring of Staphylococcus aureus, Streptococcus, Escherichia coli, Pseudomonas aeruginosa, Acinetobacter baumannii, Enterococcus faecium and/or Clostridium difficile infections.

### BACKGROUND OF THE INVENTION

Despite great advances in the treatment and prevention of bacterial infections, they remain a significant cause of illness and death in both clinical and non-clinical settings. Staphylococcus aureus (S. aureus), Streptococcus, Escherichia coli (E. coli), Pseudomonas aeruginosa (P. aeruginosa), Acinetobacter baumannii (A. baumannii), Enterococcus faecium (E. faecium), and Clostridium difficile (C. difficile) account for a significant portion of infections in the U.S. and abroad. A S. aureus infection can cause a broad range of illnesses from minor skin infections, such as atopic dermatitis, impetigo, boils, cellulitis, folliculitis, furuncles, carbuncles, scalded skin syndrome and abscesses, to life-threatening diseases such as bacteremia (bacterial infection of the bloodstream), pneumonia, meningitis, osteomyelitis, endocarditis, staphylococcal toxic shock syndrome (TSS) and septicemia. A Streptococcus infection can similarly lead to a number of serious conditions, such as bacteremia, pneumonia, meningitis, pharyngitis (“strep throat”), otitis media, scarlet fever, acute rheumatic fever, cellulitis, endocarditis, streptococcal TSS and perinatal Group B streptococcal disease. An E. coli infection can produce pneumonia, gastroenteritis, a urinary tract infection, neonatal meningitis, hemolytic-uremic syndrome (HUS), peritonitis, mastitis and septicemia. A P. aeruginosa infection commonly affects immunocompromised patients, such as those with cystic fibrosis or AIDS. Infection can affect many different parts of the body, but typically target the respiratory tract (e.g., patients with cystic fibrosis or those on mechanical ventilation), causing bacterial pneumonia. In addition to pneumonia, P. aeruginosa can cause bacteremia, septicemia, a urinary tract infection, a gastrointestinal infection, ear and eye infections, a chronic lung infection, endocarditis, dermatitis and osteochondritis. It is the most common cause of infections in burn victims. Multidrug-resistant A. baumannii is a common problem in many hospitals in the U.S. and Europe. An A. baumannii infection can cause nosocomial pneumonia and various other infections, such as skin and wound infections, bacteremia and meningitis. Severe clinical disease caused by A. baumannii bacteremia is reported to be associated with a high mortality rate of up to 75%. An Enterococcus can cause urinary tract infections, bacteremia, bacterial endocarditis, diverticulitis, and meningitis. Some Enterococci are resistant to β-lactam-based antibiotics (some penicillins and virtually all cephalosporins) as well as many aminoglycosides. Certain virulent strains of Enterococcus that are resistant to vancomycin have caused nosocomial infections of hospitalized patients especially in the US and other developed countries. A C. difficile infection is a common cause of colitis and the most significant cause of pseudomembranous colitis, a severe infection of the colon often resulting after normal gut flora is eradicated by excessive use of antibiotics. In addition to colitis and pseudomembranous colitis, a C. difficile infection may cause severe diarrhea, toxic megacolon, intestinal perforation and even death. A C. difficile infection presents particularly high risk to the elderly and individuals who require prolonged use of antibiotics, such as patients who are immunocompromised, have recently undergone gastrointestinal surgery, or have a serious underlying illness.

One of the most troubling aspects of S. aureus, Streptococcus, E. coli, P. aeruginosa, A. baumannii, E. faecium and C. difficile infections is the recent proliferation of bacterial strains that are resistant to a broad spectrum of antibiotics. For example, a 2007 report by the U.S. Centers for Disease Control and Prevention (CDC) estimated that the number of methicillin-resistant S. aureus (MRSA) infections treated in hospitals doubled nationwide, from approximately 127,000 in 1999 to 278,000 in 2005, while the number of deaths increased from 11,000 to more than 17,000 at the same time. See Klein et al., Emerg. Infect. Dis. 2007, 13:1840-1846. Another recent CDC study estimated that MRSA was responsible for 94,360 serious infections and was associated with 18,650 hospital stay-related deaths in the United States in 2005. See Klevens et al., J.A.M.A. 2007, 298:1763-1771; CDC Features, “MRSA: Methicillin-resistant Staphylococcus aureus in Healthcare Settings,” Oct. 17, 2007. Similarly, active vaccination strategies are not always effective because of the constant evolution of new bacterial strains that do not express the antigens used to induce immune response in a vaccinated individual. Moreover, active immunization takes time to achieve its full effect, whereas many acute S. aureus, Streptococcus, E. coli, P. aeruginosa, A. baumannii, E. faecium and C. difficile infections require immediate intervention. A vaccination is designed to be effective against the particular bacterial strain(s) selected by the vaccine maker. At least in certain embodiments, the present antibodies and methods are designed to be effective against endogenous flora of bacterial strain(s) that the individual is exposed to.

Antibody-based therapeutics have a number of advantages over other immune-modulating strategies such as vaccines because antibodies function immediately upon administration, irrespective of whether the patient has a fully functional immune system. Since their first administration in the form of antisera in the 1890s, they have come a long way with the development of monoclonal antibodies (mAbs), antibody fragments, domain antibodies and polyclonal antibodies today. The original infusion of immunoglobulins extracted from human plasma had the advantage of reflecting the natural immune response, relating to the breadth of its repertoire and its diversity. However, several limitations including scarcity of suitable immune plasma, batch-to-batch variation, cost and safety issues have prevented the widespread use of immunoglobulin therapy in its original form.

The development of the hybridoma technique revolutionized the antibody field. This technique allows virtually unlimited production of pure, highly specific monoclonal antibodies in vitro. mAbs have a number of disadvantages, however, which are related to their narrow specificity. Their effects do not cover the full spectrum of effector mechanisms of a natural immune response and mAbs are, therefore, less effective in the treatment of diseases that have complex target antigens. In cases of antigen mutation, or when facing a disease caused by a pathogen with multiple strains, mAbs can also become ineffective. In addition, in spite of efforts to humanize the monoclonal antibodies, there is still a problem with induction of human antibodies against the therapeutic monoclonal antibodies leading to inactivation of the therapeutic monoclonal antibodies and risk of anaphylaxis.

The so-called multi-hit theory teaches that neutralization of a given pathogen depends primarily on achieving a sufficient antibody density on the pathogen's surface and less on the specific epitopes utilized. Since mAbs inherently target a single epitope, pathogen-specific mAbs may, even at high concentrations, be unable to provide a sufficient antibody coating density to mediate bacterial neutralization or elimination, including neutralization or elimination of bacterial toxins. Under normal conditions, the diversity of the human antibody repertoire comprises antibodies against multiple epitopes on the pathogen's surface, thereby securing sufficient antibody coverage to neutralize and eliminate the pathogen. Additionally, the polyclonal nature of the human antibody response reduces the likelihood of immune escape, since a bacterial cell would need to simultaneously acquire escape mutations in several, if not all of the targeted epitopes.

Early beginnings of passive antibody therapy involved the purification of the immunoglobulin fraction of human donor plasma and its infusion into patients. Plasma-derived immunoglobulin from normal healthy donors offers the advantage of mimicking the polyclonal natural immune response with a diverse and specific repertoire, and remains a preferred choice in the treatment of selected conditions. Plasma-derived immunoglobulins reflect the breadth of the human antibody repertoire and, yet, the specificity of the antibody response, with the presence of several antibodies against the pathogen's multiple epitopes increasing the chance of triggering effector mechanisms.

Deriving immunoglobulin from whole human plasma, reflecting the multitude of binding specificities in the natural antibody, implies that only a small fraction of all the immunoglobulin injected is targeting the antigen of interest. This can be partially overcome by the injection of hyperimmune immunoglobulin-derived from individuals who have developed a high titre of antibodies against certain disease-related antigens following (for instance) recovery from infection. Today, hyperimmune immunoglobulin is used for prophylaxis or therapy against infections with hepatitis B virus, respiratory syncytial virus (RSV), cytomegalovirus (CMV) and rabies virus, as well as tetanus, botulinum intoxication and Rhesus D (RhD) alloimmunization.

A more widespread use of immunoglobulin products has been prevented by the fact that the products are highly dependent on donor blood availability, both in terms of quantity and suitability, resulting in considerable variation between batches. Additionally, since only a small fraction of immunoglobulins are specific to the bacterial pathogens of interest, e.g., bacterial toxins, a relatively large amount of immunoglobulins must be administered to a patient in order to achieve the desired bacterial neutralization. Given the advantages of polyclonal antibodies in the immunity to bacteria, bacterial toxins, and the challenges associated with developing effective mAb-based drugs to most bacterial infections, technologies to identify and produce more complex antibody compositions have been developed. Thus, the combination of two or more mAb into cocktails has been attempted, and this approach may in some cases circumvent limitations associated with anti-viral mAb products. However, the cost associated with production and characterization of separate batches of individual mAb components may limit the number of antibodies feasibly included in such cocktails and thereby possibly their efficacy and applicability. Alternative strategies to overcome these challenges rely on using animals such as cows transgenic for human antibody genes for production of plasma-derived polyclonal antibodies after immunization with a given pathogen. Although these technologies appear promising, they suffer from the reduced specific activity due to the presence of a predominance of irrelevant antibody molecules, the need for knocking-out the animal's endogenous antibody genes, and the risk of transferring zoonosis or prions to the recipient.

Norrby et al., Infect. Immun., 64(12):5395-8 (1996), demonstrated that normal polyspecific immunoglobulin given intravenously (IVIG) and plasma samples from patients treated with IVIG neutralize the mitogenic and cytokine-inducing activities of group A streptococcal (GAS) superantigens. Norrby et al. investigated whether this neutralizing activity is mediated by antibodies to these superantigens. IVIG and plasma samples collected from a patient with GAS necrotizing fasciitis post-IVIG infusions markedly inhibited the mitogenic activity elicited by the streptococcal pyrogenic exotoxins SpeB and SpeC, as well as by GAS culture supernatant Immunoblot analysis showed marked increases in the levels of antibodies to SpeC and proteins in the GAS culture supernatant in post-IVIG over those of pre-IVIG plasma samples. Removal of antisuperantigen antibodies in IVIG by adsorption to SpeC- and GAS culture supernatant-coupled Sepharose markedly reduced the neutralizing ability of IVIG against respective stimuli. The neutralizing activity was totally recovered in the eluted antibodies. By contrast, although pre- and post-IVIG plasma samples contained antibodies to SpeA, these antibodies did not block the activity of this superantigen. Nonspecific immunomodulatory activity of IVIG was ruled out because neither the IVIG nor the affinity-purified antibodies significantly inhibited the response to the polyclonal T-cell mitogen phytohemagglutinin A. Norrby et al. stated that these data provide direct evidence that the neutralizing activity in IVIG and in patient plasma samples following IVIG treatment is mediated by antibodies to superantigens and indicate that the quality rather than the quantity of these antibodies may be more clinically relevant.

LeClaire and Bavari, Antimicrob. Agents Chemother., 45(2):460-3 (2001), stated that bacterial superantigens (BSAgs) cause massive stimulation of the immune system and are associated with various pathologies and diseases. To address the role of antibodies in protection against BSAgs, LeClaire and Bavari screened the sera of 29 human volunteers for antibodies to the SAgs staphylococcal enterotoxin A (SEA), SEB, SEC1, and toxic shock syndrome toxin 1 (TSST-1). Although all volunteers had detectable levels of antibodies against SEB and SEC1, many (9 out of 29 volunteers) lacked detectable antibody to SEA or had minimal titers. Antibody titers to TSST-1 were well below those to SEB and SEC1, and three volunteers lacked detectable antibody to this BSAg. In addition, pooled immunoglobulin preparations obtained from different companies had antibody titers against SEs and TSST-1. There was a good correlation between antibody titers and inhibition of superantigenic effects of these toxins. Transfer of SEB-specific antibodies, obtained from pooled sera, suppressed in vitro T-cell proliferation and totally protected mice against SEB. LeClaire and Bavari stated that these data suggest that the inhibitory activity of human sera was specific to antibodies directed against the toxins. LeClaire and Bavari also stated that it may be possible to counteract with specific antibodies BSAg-associated pathologies caused by stimulation of the immune system.

Horwith et al., U.S. Patent Publication No. 2006/0153857 A1, is directed to a method of preventing or treating bacteremia caused by Staphylococcus aureus, comprising administering a monoclonal or polyclonal antibody composition comprising antibodies specific for one or more S. aureus antigens. In one specific embodiment, the composition is a hyperimmune specific IGIV composition. In another specific embodiment, the composition comprises antibodies to a capsular polysaccharide S. aureus antigen, such as the Type 5 and/or Type 8 antigens. In another embodiment, the composition comprises monoclonal antibodies to a capsular polysaccharide S. aureus antigen. Horwith et al. stated that this method provides an effective tool for preventing or treating S. aureus bacteremia, and can be used alone or in combination with other therapies.

Thus, no fully effective solution has been found for the prevention, removal, treatment and monitoring of S. aureus, Streptococcus, E. coli, P. aeruginosa, A. baumannii, E. faecium and/or C. difficile infections, particularly infections caused by bacterial strains that are resistant to antibiotic treatments and resistant to the antibodies generated following vaccinations. Thus, there is a need to develop new therapeutic and prophylactic, prognostic, diagnostic and treatment monitoring compositions and methods to address these problems. The present invention addressed this and other related needs.

### SUMMARY OF THE INVENTION

In one aspect, the present invention provides methods for treating, removing or preventing a bacterial infection, which comprise administering to a human suffering, suspected of suffering or at risk of suffering from Staphylococcus aureus (S. aureus) infection, a Streptococcus infection, Escherichia coli (E. coli) infection, Pseudomonas aeruginosa (P. aeruginosa) infection, Acinetobacter baumannii (A. baumannii) infection, Enterococcus faecium (E. faecium) infection and/or Clostridium difficile (C. difficile) infection an effective amount of human polyclonal antibodies affinity purified from a human blood sample with an antigenic preparation comprising cellular and/or secreted antigen(s) from bacterial cells selected from S. aureus, a Streptococcus, E. coli, P. aeruginosa, A. baumannii, E. faecium, C. difficile or a combination of any of these bacteria. Preferably, the affinity purified human polyclonal antibodies are purified (e.g., as made more concentrated as compared to the starting or unpurified material) relative to the same human polyclonal antibodies in the unpurified or non-affinity-purified human blood sample, e.g., intravenous immunoglobulin (IVIG) sample. Also preferably, the affinity purified human polyclonal antibodies are specific for the bacterial antigen(s) used in the affinity purification. Further preferably, the affinity purified human polyclonal antibodies are substantially free of human antibodies that specifically bind to non-bacterial antigens in the human blood sample.

In some embodiments, the antigenic preparations of the present invention may comprise a whole cell extract and/or secreted antigen(s) of S. aureus, a Streptococcus, E. coli, P. aeruginosa, A. baumannii, E. faecium and/or C. difficile.

The antigenic preparations of the present invention may be obtained by any suitable methods, or combination of suitable methods. In some embodiments, antigenic preparations of the present invention may be obtained by a method comprising the following steps. First, bacterial cells are grown in a protein containing culture medium for a specified period of time to a desired cell density. Then, the bacterial cells are collected, resuspended in a non-protein containing culture medium and grown in that non-protein containing culture medium for a specified period of time. Then, the bacterial cells are disrupted in order to collect a whole cell extract from the disrupted cells. Antigens secreted by the bacterial cells are also collected from the non-protein containing culture medium and combined with the whole cell extract to yield the antigenic preparation.

The present invention further contemplates the use of affinity purified human polyclonal antibodies to assess the suitability of a human subject for the therapeutic, removal or preventive treatment, to monitor the efficacy of the therapeutic, removal or preventive treatment or to determine an optimal therapeutic or preventive dose of the affinity purified human polyclonal antibodies.

In some embodiments, the present methods may comprise, prior to administering the affinity purified human polyclonal antibodies to the human, conducting an immunotest to determine the presence, absence and/or amount of bacterial antigens in a blood sample of the human using the same affinity purified human polyclonal antibodies, to assess the suitability of the human for the therapeutic, removal or preventive treatment, wherein a positive immunotest result indicates that the human is suitable for therapy, removal or prevention of bacterial infection using the affinity purified human polyclonal antibodies.

In some embodiments, the present methods may comprise, before and after administering the affinity purified human polyclonal antibodies to the human, conducting an immunotest to determine the presence, absence and/or amount of bacterial antigens in a blood sample of the human using the same affinity purified human polyclonal antibodies, to monitor the efficacy of the therapeutic, removal or preventive treatment, wherein the absence or reduction in the bacterial antigens after administering the affinity purified human polyclonal antibodies to the human relative to the amount of bacterial antigens before the administration indicates efficacy of the therapeutic, removal or preventive treatment.

In some embodiments, the present methods may comprise, before and after administering the affinity purified human polyclonal antibodies to the human, conducting an immunotest to determine the presence, absence and/or amount of bacterial antigens in a blood sample of the human using the same affinity purified human polyclonal antibodies, to determine an optimal therapeutic, removal or preventive dose of the affinity purified human polyclonal antibodies, wherein the optimal therapeutic, removal or preventive dose is determined based on the amount of the bacterial antigens remaining after administering the affinity purified human polyclonal antibodies to the human and the extent of reduction in the bacterial antigens after administering the affinity purified human polyclonal antibodies to the human relative to the amount of bacterial antigens before the administration.

In some embodiments, the present methods may comprise conducting an immunotest to determine the presence, absence and/or amount of bacterial antigens in a blood sample of the human using the same affinity purified human polyclonal antibodies to assess the suitability of the human for the therapeutic or preventive treatment, to monitor the efficacy of the therapeutic or preventive treatment or to determine an optimal therapeutic or preventive dose, wherein the antigenic preparation comprises a whole cell extract and secreted antigens of S. aureus, a Streptococcus, E. coli, P. aeruginosa, A. baumannii, E. faecium and/or C. difficile.

In some embodiments, the present methods may comprise a step of substantially inactivating and/or removing a virus. Any virus that may contaminate or compromise the therapeutic or preventive use of the affinity purified human polyclonal antibodies may be substantially inactivated and/or removed. In some embodiments, the virus to be substantially inactivated and/or removed is a lipid-enveloped or non-enveloped virus. Any suitable methods can be used to substantially inactivate and/or remove a virus. In some embodiments, a lipid-enveloped virus is substantially inactivated and/or removed by a filtration based on the virus size and a solvent/detergent treatment step, e.g., a solvent/detergent treatment step using tri-n-butyl phosphate and Triton X-100. See, e.g., Horowitz, B., “Investigations Into the Application of Tri(n-Butyl) Phosphate/Detergent Mixtures to Blood Derivatives,” Curr. Stud. Hematol. Transfus. 1989, 56:83-96; U.S. Pat. Nos. 3,962,421 and 4,540,573, all of which are incorporated herein by reference in their entireties.

The present invention further provides pharmaceutical compositions for treating or preventing a bacterial infection, which comprise an effective amount of human polyclonal antibodies affinity purified from a human blood sample with an antigenic preparation comprising cellular and/or secreted antigen(s) from S. aureus, a Streptococcus, E. coli, P. aeruginosa, A. baumannii, E. faecium, C. difficile or a combination of these bacterial cells. Preferably, the affinity purified human polyclonal antibodies are purified (e.g., as made more concentrated as compared to the starting or unpurified material) relative to the same human polyclonal antibodies in the unpurified or non-affinity-purified human blood sample, e.g., intravenous immunoglobulin (IVIG) sample. Also preferably, the affinity purified human polyclonal antibodies are specific for the bacterial antigen(s) used in the affinity purification. Further preferably, the affinity purified human polyclonal antibodies are substantially free of human antibodies that specifically bind to non-bacterial antigens in the human blood sample.

In some embodiments, the pharmaceutical compositions may also comprise one or more pharmaceutically acceptable carrier or excipient. In some embodiments, the pharmaceutical compositions may further comprise one or more additional therapeutic or preventive agent.

The present invention further provides additional methods for treating or preventing a bacterial infection, which comprise administering to a human suffering, suspected of suffering or at risk of suffering from Staphylococcus aureus (S. aureus) infection, a Streptococcus infection, Escherichia coli (E. coli) infection, Pseudomonas aeruginosa (P. aeruginosa) infection, Acinetobacter baumannii (A. baumannii) infection, Enterococcus faecium (E. faecium) infection and/or Clostridium difficile (C. difficile) infection an effective amount of the pharmaceutical composition according to the present invention.

### BRIEF DESCRIPTION OF THE DRAWINGS

FIGS. 1A-C show growth charts of Staphylococcus aureus, Streptococcus pyogenes and Escherichia coli cultures, respectively, in protein-containing and protein-free media.

FIG. 2 shows the HPLC profile of a Staphylococcus aureus antigenic preparation according to the present invention.

FIG. 3 shows the HPLC profile of a Streptococcus pyogenes antigenic preparation according to the present invention.

FIG. 4 shows the HPLC profile of an Escherichia coli antigenic preparation according to the present invention.

FIG. 5 shows the HPLC profile of a combined antigenic preparation of Staphylococcus aureus, Streptococcus pyogenes and Escherichia coli according to the present invention.

FIG. 6 shows the HPLC profile of affinity-purified human polyclonal antibodies against Staphylococcus aureus toxin A (SEA).

FIG. 7 shows the HPLC profile of affinity-purified human polyclonal antibodies against Staphylococcus aureus toxin B (SEB).

FIG. 8 shows the HPLC profile of affinity-purified human polyclonal antibodies against an Escherichia coli antigenic preparation.

FIG. 9 shows titers of affinity-purified human polyclonal antibody preparations against A. baumannii, P. aeruginosa and S. aureus.

### DETAILED DESCRIPTION OF THE INVENTION

### I. Definitions

Unless defined otherwise, all technical and scientific terms used herein have the same meaning as is commonly understood by one of ordinary skill in the art to which this invention belongs. All patents, patent applications (published or unpublished), and other publications referred to herein are incorporated by reference in their entireties. If a definition set forth in this section is contrary to or otherwise inconsistent with a definition set forth in the patents, applications, published applications and other publications that are incorporated herein by reference, the definition set forth in this section prevails over the definition that is incorporated herein by reference.

As used herein, “a” or “an” means “at least one” or “one or more.”

As used herein, the term “treating or preventing” refers to any and all uses which remedy or prevent a diseased or infected state or symptoms, or otherwise deter, hinder, retard, or reverse the progression of a disease/infection or other undesirable symptoms. As used herein, the terms “treating” and “therapeutic” refer to any improvement or amelioration of any consequence of disease; full eradication of disease is not required. Amelioration of symptoms of a particular disorder refers to any lessening of symptoms, whether permanent or temporary, that can be attributed to or associated with administration of a therapeutic composition of the present invention.

As used herein, the terms “administration” or “administering” refers to any suitable method of providing a composition of the present invention of the invention to a subject. It is not intended that the present invention be limited to particular modes of administration. The affinity purified polyclonal human antibodies and pharmaceutical compositions of the present invention may be administered by oral, parenteral (e.g., intramuscular, intraperitoneal, intravenous, intracisternal injection or infusion, subcutaneous injection, or implant), inhalation spray, nasal, vaginal, rectal, sublingual, or topical routes of administration. The pharmaceutical compositions may be formulated in suitable dosage unit formulations appropriate for each route of administration.

As used herein, the term “effective amount” or “therapeutically effective amount” of an active agent refers to a nontoxic but sufficient amount of the agent to provide the desired therapeutic or prophylactic effect to most patients or individuals. It is commonly recognized that the effective amount of a pharmacologically active agent may vary depending on the route of administration, as well as the age, weight, and sex of the individual to which the drug or pharmacologically active agent is administered. It is also commonly recognized that one of skill in the art can determine appropriate effective amounts by taking into account such factors as metabolism, bioavailability, and other factors that affect plasma levels of an active agent following administration within the unit dose ranges disclosed further herein for different routes of administration.

As used herein, the term “antibody” refers to monoclonal and polyclonal antibodies, whole antibodies, antibody fragments, and antibody sub-fragments that exhibit specific binding to a specific antigen of interest. Thus, “antibodies” can be whole immunoglobulin of any class, e.g., IgG, IgM, IgA, IgD and IgE. The ability of a given molecule, including an antibody fragment or sub-fragment, to act like an antibody and specifically bind to a specific antigen can be determined by binding assays known in the art, for example, using the antigen of interest as the binding partner.

As used herein, the term “specific binding” refers to the specificity of an antibody such that it preferentially binds to a defined target, such as a cellular and/or secreted bacterial antigen. Recognition by an antibody of a particular target in the presence of other potential targets is one characteristic of such binding. Preferably, antibodies or antibody fragments that are specific for or bind specifically to a bacterial antigen bind to the target bacterial antigen with higher affinity than binding to other non-target antigens. Also preferably, antibodies or antibody fragments that are specific for or bind specifically to a bacterial antigen avoid binding to a significant percentage of non-target and/or non-bacterial antigens, e.g., substances used in the preparation of the bacterial antigens. In some embodiments, antibodies or antibody fragments of the present disclosure avoid binding greater than about 90% of non-target and/or non-bacterial antigens, although higher percentages are clearly contemplated and preferred. For example, antibodies or antibody fragments of the present disclosure avoid binding about 91%, about 92%, about 93%, about 94%, about 95%, about 96%, about 97%, about 98%, about 99%, and about 99% or more of non-target and/or non-bacterial antigens. In other embodiments, antibodies or antibody fragments of the present disclosure avoid binding greater than about 10%, 20%, 30%, 40%, 50%, 60%, or 70%, or greater than about 75%, or greater than about 80%, or greater than about 85% of non-target and/or non-bacterial antigens.

As used herein, the term “polyclonal antibodies” refers to a heterogeneous population of antibody molecules that bind to different antigens and/or different epitopes of the same antigen. More specifically, the polyclonal antibodies of the present invention bind to different cellular and secreted antigens of S. aureus, Streptococcus, E. coli, P. aeruginosa, A. baumannii, E. faecium and/or C. difficile cells.

The mixture of polyclonal antibodies includes polyclonal antibodies from a plurality of different subjects. In some contexts, the terms “individual,” “host,” “subject,” and “patient” are used interchangeably to refer to an animal that is the object of treatment, observation and/or experiment. “Animal” includes vertebrates and invertebrates, such as fish, shellfish, reptiles, birds, and, in particular, mammals. “Mammal” includes, without limitation, mice, rats, rabbits, guinea pigs, dogs, cats, sheep, goats, cows, horses, primates, such as monkeys, chimpanzees, and apes, and, in particular, humans. In some embodiments, the polyclonal antibodies are derived from the blood, plasma or sera of human subjects.

In some embodiments, the mixture of polyclonal antibodies can be obtained from 2, 3, 4, 5, 6, 7, 8, 9, 110, 11, 12, 13, 14, 15, 16, 17, 28, 19, 20, 21, 22, 23, 24, 25, 26, 27, 28, 29, 30, 35, 40, 45, 50 55, 60, 65, 70, 75, 80, 85, 90, 95, 100, or more, individual subjects, or any number in between. In some embodiments, all of the individual subjects from whom the pool of polyclonal antibodies is obtained are infected with the target pathogenic organism. In other embodiments, some, but not all of the subjects from whom the pool of polyclonal antibodies are obtained are infected with the target pathogen. In some embodiments, none of the individuals show symptoms or clinical indications of being infected with the target pathogen. In some embodiments, some or all of the individuals have been exposed to the target pathogenic organism, but do not show the symptoms or clinical indications of being infected with the target pathogenic organism. As used herein, an individual “infected with” a target pathogen refers to individuals in which the target pathogen is present. As used herein, an individual that has been “exposed to” a target pathogen refers to an individual that was at one point in time infected with a target pathogen, but in whom the target pathogen is not necessarily still present. As discussed further below, routine diagnostic tests can be used to determine whether an individual is infected with, or has been exposed to, a target pathogen. Preferably, all or almost all of the individuals from whom the polyclonal antibodies are obtained have mounted an immune response against the target pathogen, and, as such, have plasma that contains a detectable concentration of target-specific antibodies.

As used herein, the term “antigen” refers to a target molecule that is specifically bound by an antibody through its antigen recognition site. The antigen may be monovalent or polyvalent, i.e. it may have one or more epitopes recognized by one or more antibodies. Examples of kinds of antigens that can be recognized by antibodies include polypeptides, oligosaccharides, glycoproteins, polynucleotides, lipids, etc.

As used herein, the term “epitope” refers to a polypeptide sequence of at least about 3 to 5, preferably about 5 to 10 or 15, and not more than about 1,000 amino acids (or any integer there between), which define a sequence that by itself or as part of a larger sequence, binds to an antibody generated in response to such sequence. There is no critical upper limit to the length of the fragment, which may, for example, comprise nearly the full-length of the antigen sequence, or even a fusion protein comprising two or more epitopes from the target antigen. An epitope for use in the subject invention is not limited to a polypeptide having the exact sequence of the portion of the parent protein from which it is derived, but also encompasses sequences identical to the native sequence, as well as modifications to the native sequence, such as deletions, additions and substitutions (generally conservative in nature).

As used herein, the term “non-bacterial antigen” refers to a target molecule of non-bacterial origin. More specifically, the term “non-bacterial antigen” refers to a protein, peptide, oligosaccharide, glycoprotein, polynucleotide or lipid that is not derived from S. aureus, Streptococcus, E. coli, P. aeruginosa, A. baumannii, E. faecium and/or C. difficile cells. In some embodiments, “non-bacterial antigen” refers to a mammalian antigen, particularly a human antigen.

As used herein, the term “S. aureus” refers to a pathogenic strain of Staphylococcus aureus, including antibiotic resistant strains, such as methicillin resistant strains (MRSA) and vancomycin resistant strains (VISA and VRSA). In some embodiments, “S. aureus” refers to a strain that is resistant to more than one antibiotic. In some embodiments, the term “S. aureus” refers to the methicillin resistant strains USA300 (also known as FPR 3757; ATCC #BAA-1556) and NYBK2464 (ATCC #BAA-51).

As used herein, the term “Streptococcus” refers to a pathogenic strain of Streptococcus pneumoniae, Group A Streptococcus (GAS; e.g., Streptococcus pyogenes) and Group B Streptococcus (GBS; e.g., Streptococcus agalactiae), including antibiotic-resistant strains, such as S. pneumoniae strains resistant to penicillin, tetracycline, clindamycin, a cephalosporin, a macrolide or a quinolone. In some embodiments, “Streptococcus” refers to the GAS strain ATCC #19615 and the GBS strain ATCC #25663.

As used herein, the term “E. coli” refers to a pathogenic strain of Escherichia coli, including antibiotic resistant strains, such as E. coli strains resistant to penicillin, streptomycin, chloramphenicol, ampicillin, cephalosporin or tetracycline. As used herein, “E. coli” encompasses enterotoxigenic E. coli (ETEC), enteropathogenic E. coli (EPEC), enteroinvasive E. coli (EIEC), enterohemorrhagic E. coli (EHEC), enteroaggregative E. coli (EAggEC) and uropathogenic E. coli (UPEC). In some embodiments, “E. coli” refers to a Shiga toxin-producing E. coli (STEC), such as the strain O157:H7 (ATCC #43895).

As used herein, the term “P. aeruginosa” refers to a pathogenic strain of Pseudomonas aeruginosa, including antibiotic-resistant strains, such as P. aeruginosa strains resistant to beta-lactams antibiotics (e.g., penicillin), piperacillin, imipenem, tobramycin or ciprofloxacin. In some embodiments, “P. aeruginosa” may refer to the strains identified as ATCC #9027, ATCC #10145 or ATCC #15442. In some embodiments, the term “P. aeruginosa” refers to a pathogenic strain that infects cystic fibrosis patients.

As used herein, the term “A. baumannii” refers to a pathogenic strain of Acinetobacter baumannii, including any antibiotic-resistant strains, such as A. baumannii strains resistant to ceftazidime, gentamicin, ticarcillin, piperacillin, aztreonam, cefepime, ciprofloxacin, imipenem or meropenem. In some embodiments, “A. baumannii” may refer to the strain identified as ATCC #BAA-1605.

As used herein, the term “E. faecium” refers to a pathogenic strain of Enterococcus faecium, including antibiotic-resistant strains, such as E. faecium strains resistant to β-lactam-based antibiotics (e.g., penicillins and cephalosporins) or aminoglycosides. In some embodiments, “E. faecium” may refer to the strain identified as ATCC #51559.

As used herein, the term “C. difficile” refers to a pathogenic strain of Clostridium difficile, including any antibiotic-resistant strains. In some embodiments, “C. difficile” may refer to the strains identified as ATCC #9689 or ATCC #BAA-1382.

As used herein, the term “antigenic preparation comprising cellular and secreted antigens” refers to a preparation comprising any antigens secreted by S. aureus, Streptococcus, E. coli, P. aeruginosa, A. baumannii, E. faecium and/or C. difficile cells and/or any cellular, e.g., soluble, antigens produced by the disruption of such cells using any physical and/or chemical means. Thus, the term encompasses soluble bacterial cell extracts, including whole cell extracts or cell surface or membrane extracts. In some embodiments, the “antigenic preparation” does not include intact bacterial cells or insoluble particulate matter, such as bacterial walls or bacterial nuclei. In some embodiments, the antigenic preparation may comprise secreted bacterial toxin(s), oligosaccharide(s), protein(s), peptide(s), lipid(s), and other soluble cellular component(s). In some embodiments, the antigenic preparation may comprise secreted toxin(s), oligosaccharide(s), protein(s), peptide(s) and glycoprotein(s) from S. aureus, a Streptococcus, E. coli, P. aeruginosa, A. baumannii, E. faecium and/or C. difficile. In some embodiments, the “antigenic preparation” comprises a single cellular antigen and/or a single secreted antigen. In other embodiments, the “antigenic preparation” comprises a single cellular antigen and/or multiple secreted antigens. In still other embodiments, the “antigenic preparation” comprises multiple cellular antigens and/or a single secreted antigen. In yet other embodiments, the “antigenic preparation” comprises multiple cellular antigens and/or multiple secreted antigens.

As used herein, the term “whole cell extract” refers to any cellular components of S. aureus, Streptococcus, E. coli, P. aeruginosa, A. baumannii, E. faecium and/or C. difficile cells that remain in extraction solution, e.g., an aqueous solution or a non-aqueous solution, following a physical or chemical disruption of the bacterial cells. “Whole cell extract” is not meant to encompass intact bacterial cells and insoluble components, such as bacterial walls and nuclei that can be removed from the extraction solution by any suitable methods, such as filtration or centrifugation. In some embodiments, the whole cell extract may contain soluble proteins, glycoproteins, peptides, oligosaccharides, lipids, polynucleotides from S. aureus, a Streptococcus, E. coli, P. aeruginosa, A. baumannii, E. faecium and/or C. difficile.

As used herein, the term “human blood sample” refers to whole blood, plasma or serum obtained from one or more human subjects. “Whole blood” refers to the fluid and cellular portion of the plasma in circulating blood. “Plasma” refers to the fluid, non-cellular portion of the blood, distinguished from the serum obtained after coagulation. “Serum” refers to the fluid portion of the blood obtained after removal of the fibrin clot and blood cells, distinguished from the plasma in circulating blood. In some embodiments, “human blood sample” refers to a serum sample obtained from a normal human subject. In some embodiments, serum samples from multiple human subjects, preferably normal humans, are pooled in order to generate greater diversity of polyclonal antibodies.

As used herein, the term “normal human (or healthy individual)” refers to a human subject that is not hyperimmune to S. aureus, a Streptococcus, E. coli, P. aeruginosa, A. baumannii, E. faecium and/or C. difficile as a result of vaccination against S. aureus, a Streptococcus, E. coli, P. aeruginosa, A. baumannii, E. faecium and/or C. difficile, especially recent vaccination against S. aureus, a Streptococcus, E. coli, P. aeruginosa, A. baumannii, E. faecium and/or C. difficile or recent exposure to an acute S. aureus, a Streptococcus, E. coli, P. aeruginosa, A. baumannii, E. faecium and/or C. difficile bacterial infection, especially the infection that led to bacteremia.

As used herein, the term “substantially free of human antibodies that specifically bind to non-bacterial antigens” refers to a composition of affinity purified polyclonal human antibodies that contains no more than about 90%, 80%, 70%, 60%, 50%, 40%, or 30%, preferably no more than about 20%, more preferably no more than about 10% and most preferably no more than about 5% of antibodies that specifically bind to non-bacterial antigens. As explained above, the term “non-bacterial antigens” as used herein usually refers to polypeptides, oligosaccharides, glycoproteins, polynucleotides or lipids derived from sources other than S. aureus, Streptococcus, E. coli, P. aeruginosa, A. baumannii, E. faecium and/or C. difficile cells.

As used herein, “bacteremia” refers to the presence of viable bacteria and/or bacterial toxin(s) in the bloodstream of a human subject. “Bacteremia caused by S. aureus” or “S. aureus bacteremia” refers to bacteremia in which at least some of the bacteria in the blood are S. aureus. Other bacterial species, such as a Streptococcus, E. coli, P. aeruginosa, A. baumannii, E. faecium and/or C. difficile, also may be present in the bloodstream.

As used herein, the term “substantially removed in the antigenic preparation” generally refers to an antigenic preparation in which more than about 10%, 20%, 30%, 40%, 50%, 60%, 70%, preferably more than about 80%, more preferably more than about 90% and most preferably more than about 95% of a recited component has been removed. For example, the phrase “S. aureus Protein A is substantially removed in the antigenic preparation” means that more than about 70%, preferably more than about 80%, more preferably more than about 90% and most preferably more than about 95% of Protein A has been removed. Because S. aureus Protein A is a gamma globulin (IgG) binding protein which binds to the non-variable Fc region of an antibody, its effective removal is important to ensure that the antigenic preparation is substantially free of human antibodies that specifically bind to non-bacterial antigens.

As used herein, the phrase “substantially inactivating and/or removing a virus” generally refers to an antigenic preparation in which more than about 10%, 20%, 30%, 40%, 50%, 60%, 70%, preferably more than about 80%, more preferably more than about 90% and most preferably more than about 95% of a recited or target virus has been removed.

As used herein, the term “capsular polysaccharide” refers to a layer of polysaccharide external to but contiguous with the cell wall of a microorganism. Capsular polysaccharides are distinct from lipopolysaccharides (LPS) and the polysaccharides derived therefrom. The term “lipopolysaccharide” is commonly used to refer to the endotoxic component of the outer membrane in Gram negative bacteria.

As used herein, the terms “Type 5 antigen,” “Type 8 antigen” and “336 antigen” refer to S. aureus antigens that are present in most cases of S. aureus bacteremia. Type 5 and Type 8 antigens are capsular polysaccharide antigens that usually comprise a polysaccharide backbone bearing O-acetyl groups. Type 5 and Type 8 S. aureus antigens are described in Fattom et al., Infect. Immun. 1990, 58:2367-2374 and Fattom et al., Infect. Immun. 1996, 64:1659-1665. The 336 antigen is another common S. aureus antigen, which is described in U.S. Pat. No. 6,537,559.

As used herein, the term “toxin” refers to any cytotoxic molecule secreted from bacterial cells or associated with the bacterial cell wall. The secreted toxins are commonly referred to as “exotoxins,” and the cell-associated toxins are referred to as “endotoxins.” Most endotoxins are located in the cell envelope. As used herein, endotoxins refer specifically to the lipopolysaccharide (LPS) or lipooligosaccharide (LOS) located in the outer membrane of Gram-negative bacteria. Although they are structural components of bacterial cells, soluble endotoxins may be released from growing bacteria or from cells that are lysed as a result of host defense mechanisms or by the activities of certain antibiotics. Endotoxins generally act in the vicinity of bacterial growth or presence. In contrast, exotoxins are usually secreted by bacteria and act at a site removed from bacterial growth. However, in some cases, exotoxins are only released by lysis of the bacterial cell. Exotoxins are usually proteins or polypeptides that act enzymatically or through direct action with host cells and stimulate a variety of responses.

As used herein, the term “protein containing culture medium” refers to any suitable bacterial growth medium that contains a protein, peptide and/or amino acid nutrient, such as a yeast extract, tryptone, casein peptone, and the like. As used herein, a protein containing culture medium is used to grow bacterial cells to a desired density, after which it is substituted with a protein-free culture medium in order to avoid the binding of human antibodies to non-bacterial antigens associated with the protein-containing culture medium. In some embodiments, the term “protein containing culture medium” refers to Bacto™ Tryptic Soy Broth containing 17.0 g/L pancreatic digest of casein; 3.0 g/L enzymatic digest of soybean meal, 5.0 g/L NaCl, 2.5 g/L K₂HPO₄ and 2.5 g/L dextrose (VWR Cat. No. 90000-378; Becton Dickinson Cat. No. 211825; 30% w/v in de-ionized H₂O). In other embodiments, the term “protein containing culture medium” refers to Difco™ Reinforced Clostridial Media containing 5.0 g/L pancreatic digest of casein, 5.0 g/L proteose peptone #3, 10.0 g/L beef extract, 3.0 g/L yeast extract, 5.0 g/L NaCl, 1.0 g/L soluble starch, 5.0 g/L dextrose, 0.5 g/L cysteine HCl and 3.0 g/L sodium acetate (Becton Dickinson Cat. No. 218081; 38% w/v in de-ionized H₂O).

As used herein, the term “non-protein containing culture medium” refers to any suitable minimal bacterial growth medium that does not contain a biologically significant amount of proteins, peptides and/or amino acids. Such a minimal bacterial growth medium usually contains water, a source of carbon (e.g., a sugar such as glucose, or a less energy-rich source such as succinate) and various salts (e.g., sodium chloride, sodium phosphate). In is understood that the composition of a non-protein containing culture medium may vary depending on the bacterial species. In some embodiments, “non-protein containing culture medium” refers to a phosphate-buffered 0.9% NaCl solution (Baxter Cat. No. 2F7125) supplemented with 2 g/L D-(+)-glucose (dextrose) (Sigma Cat. No. G5146).

As used herein, the term “insoluble cellular debris” refers to those bacterial cellular components that are insoluble in an extraction solution, e.g., an aqueous solution or a non-aqueous solution, following a physical or chemical disruption of bacterial cells. Although the term typically encompasses bacterial cell wall and bacterial nuclei, it also refers to any other bacterial components that can be filtered out or precipitated from an extraction solution following a bacterial cell disruption.

As used herein, the term “precipitation or agglutination assay” refers to an immunotest format wherein the interaction between an antibody and a particular antigen results in visible precipitation or clumping. Precipitation reactions are similar in principle to agglutination reactions; they depend on the cross linking of polyvalent antigens. When the antigen is soluble, antibody and antigen form a lattice that eventually develops into a visible precipitate. When the antigen is particulate, the reaction of an antibody with the antigen can be detected by agglutination (clumping) of the antigen. It is commonly understood that both precipitation and agglutination assays can be qualitative or quantitative.

As used herein, the term “pharmaceutical excipient” refers to a material such as an adjuvant, a carrier, pH-adjusting and a buffering agent, a tonicity adjusting agent, a wetting agent, a preservative, and the like.

As used herein, the term “pharmaceutically acceptable” refers to a non-toxic, inert composition that is physiologically compatible with humans or other mammals.

As used herein, the term “pharmaceutically acceptable formulation” or “pharmaceutical composition” refers to a composition or formulation that allows for the effective distribution of a moiety or a compound, e.g., an antibody, of the invention in that physical location most suitable for their desired activity.

Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

### II. Disease Treatment and Prevention

In one aspect, the invention provides methods for treating or preventing a bacterial infection, which comprise administering to a human suffering, suspected of suffering or at risk of suffering from Staphylococcus aureus (S. aureus) infection, a Streptococcus infection, Escherichia coli (E. coli) infection, Pseudomonas aeruginosa (P. aeruginosa) infection, Acinetobacter baumannii (A. baumannii) infection, Enterococcus faecium (E. faecium) infection and/or Clostridium difficile (C. difficile) infection, an effective amount of human polyclonal antibodies affinity purified from a human blood sample with an antigenic preparation comprising cellular and/or secreted antigen(s) from bacterial cells selected from S. aureus, a Streptococcus, E. coli, P. aeruginosa, A. baumannii, E. faecium, C. difficile, or a combination thereof. Preferably, the affinity purified human polyclonal antibodies are purified (e.g., as made more concentrated as compared to the starting or unpurified material) relative to the same human polyclonal antibodies in the unpurified or non-affinity-purified human blood sample, e.g., intravenous immunoglobulin (IVIG) sample. Also preferably, the affinity purified human polyclonal antibodies are specific for the bacterial antigen(s) used in the affinity purification. Further preferably, the affinity purified human polyclonal antibodies are substantially free of human antibodies that specifically bind to non-bacterial antigens in the human blood sample.

In some embodiments, the present methods are effective for treating the majority of the listed infections so that the combination therapy avoids the time of waiting for a time consuming diagnosis, e.g., bacterial culturing test.

In other embodiments, the affinity purified human polyclonal antibodies are concentrated, enriched or purified relative to the same human polyclonal antibodies in the unpurified or non-affinity-purified human blood sample, e.g., intravenous immunoglobulin (IVIG) sample for at least 2 fold. In one example, the specific polyclonal antibodies in the unpurified or non-affinity-purified human blood sample have a concentration of 1 mg polyclonal antibodies per 1,000 mg total antibodies, wherein 999 mg are non specific antibodies. The affinity purified human polyclonal antibodies used in the present methods have a concentration of at least 2 mg polyclonal antibodies per 1,000 mg total antibodies, wherein 998 mg are non specific antibodies. In still other embodiments, the affinity purified human polyclonal antibodies are concentrated, enriched or purified relative to the same human polyclonal antibodies in the unpurified or non-affinity-purified human blood sample, e.g., intravenous immunoglobulin (IVIG) sample for at least 5, 10, 100, 1,000, 10,000 or 50,000 fold.

The methods of the present invention overcome the narrow specificity of monoclonal antibodies by providing a wide assortment of human polyclonal antibodies specific to both secreted and cellular bacterial antigens. At the same time, the present methods also address the lack of specificity of some existing immunoglobulin preparations by providing human polyclonal antibodies that have been affinity purified with bacterial antigens to substantially exclude those antibodies that specifically bind to non-bacterial targets, thereby lowering the amount of antibodies that are required to achieve the desired therapeutic or preventive effect and reducing the likelihood of adverse side effects.

In some embodiments, the methods of the present invention may utilize an antigenic preparation comprising cellular and/or secreted antigen(s) from S. aureus. In some embodiments, the method may utilize an antigenic preparation comprising cellular and/or secreted antigen(s) from a Streptococcus. In some embodiments, the methods may utilize an antigenic preparation comprising cellular and/or secreted antigen(s) from E. coli. In some embodiments, the methods may utilize an antigenic preparation comprising cellular and/or secreted antigen(s) from P. aeruginosa. In some embodiments, the methods may utilize an antigenic preparation comprising cellular and/or secreted antigen(s) from A. baumannii. In some embodiments, the methods may utilize an antigenic preparation comprising cellular and/or secreted antigen(s) from E. faecium. In some embodiments, the methods may utilize an antigenic preparation comprising cellular and/or secreted antigen(s) from C. difficile.

In some embodiments, the methods may utilize an antigenic preparation comprising cellular and/or secreted antigens from a combination of any two bacterial species selected from S. aureus, a Streptococcus, E. coli, P. aeruginosa, A. baumannii, E. faecium and C. difficile. In some embodiments, the methods may utilize an antigenic preparation comprising cellular and/or secreted antigens from a combination of any three bacterial species selected from S. aureus, a Streptococcus, E. coli, P. aeruginosa, A. baumannii, E. faecium and C. difficile. In some embodiments, the methods may utilize an antigenic preparation comprising cellular and/or secreted antigens from a combination of any four bacterial species selected from S. aureus, a Streptococcus, E. coli, P. aeruginosa, A. baumannii, E. faecium and C. difficile. In some embodiments, the methods may utilize an antigenic preparation comprising cellular and/or secreted antigens from a combination of any five bacterial species selected from S. aureus, a Streptococcus, E. coli, P. aeruginosa, A. baumannii, E. faecium and C. difficile. In some embodiments, the methods may utilize an antigenic preparation comprising cellular and/or secreted antigens from a combination of any six bacterial species selected from S. aureus, a Streptococcus, E. coli, P. aeruginosa, A. baumannii, E. faecium and C. difficile. Alternatively, the present methods may utilize an antigenic preparation comprising cellular and/or secreted antigens from each of S. aureus, a Streptococcus, E. coli, P. aeruginosa, A. baumannii, E. faecium and C. difficile. In some embodiments, the present methods utilize an antigenic preparation comprising cellular and/or secreted antigens from each of S. aureus, S. pyogenes, S. pneumoniae, E. coli, P. aeruginosa, A. baumannii, E. faecium and C. difficile.

In another aspect, the invention provides methods for treating or preventing a bacterial infection, which comprise administering to a human suffering, suspected of suffering or at risk of suffering from Staphylococcus aureus (S. aureus) infection, a Streptococcus infection, Escherichia coli (E. coli) infection, Pseudomonas aeruginosa (P. aeruginosa) infection, Acinetobacter baumannii (A. baumannii) infection, Enterococcus faecium (E. faecium) infection and/or Clostridium difficile (C. difficile) infection, an effective amount of human polyclonal antibodies affinity purified from a human blood sample with an antigenic preparation comprising cellular and/or secreted antigens from two or more different bacterial species selected from the group consisting of S. aureus, a Streptococcus, E. coli, P. aeruginosa, A. baumannii, E. faecium and C. difficile. Preferably, the affinity purified human polyclonal antibodies are purified (e.g., as made more concentrated as compared to the starting or unpurified material) relative to the same human polyclonal antibodies in the unpurified or non-affinity-purified human blood sample, e.g., intravenous immunoglobulin (IVIG) sample. Also preferably, the affinity purified human polyclonal antibodies are specific for the bacterial antigen(s) used in the affinity purification. Further preferably, said affinity purified human polyclonal antibodies are substantially free of human antibodies that specifically bind to non-bacterial antigens in said human blood sample.

In some embodiments, the methods may utilize an antigenic preparation comprising cellular and/or secreted antigens from a combination of any two bacterial species selected from S. aureus, a Streptococcus, E. coli, P. aeruginosa, A. baumannii, E. faecium and C. difficile. For example, the antigenic preparation may comprise a secreted antigen from one bacterial species and a cellular antigen from another bacterial species, or secreted antigens from two different bacterial species, or cellular antigens from two different bacterial species. In some embodiments, the methods may utilize an antigenic preparation comprising cellular and/or secreted antigens from a combination of any three bacterial species selected from S. aureus, a Streptococcus, E. coli, P. aeruginosa, A. baumannii, E. faecium and C. difficile. In some embodiments, the methods may utilize an antigenic preparation comprising cellular and/or secreted antigens from a combination of any four bacterial species selected from S. aureus, a Streptococcus, E. coli, P. aeruginosa, A. baumannii, E. faecium and C. difficile. In some embodiments, the methods may utilize an antigenic preparation comprising cellular and/or secreted antigens from a combination of any five bacterial species selected from S. aureus, a Streptococcus, E. coli, P. aeruginosa, A. baumannii, E. faecium and C. difficile. In some embodiments, the methods may utilize an antigenic preparation comprising cellular and/or secreted antigens from a combination of any six bacterial species selected from S. aureus, a Streptococcus, E. coli, P. aeruginosa, A. baumannii, E. faecium and C. difficile. Alternatively, the present methods may utilize an antigenic preparation comprising cellular and/or secreted antigens from each of S. aureus, a Streptococcus, E. coli, P. aeruginosa, A. baumannii, E. faecium and C. difficile. In some embodiments, the present methods utilize an antigenic preparation comprising cellular and/or secreted antigens from each of S. aureus, S. pyogenes, S. pneumoniae, E. coli, P. aeruginosa, A. baumannii, E. faecium and C. difficile.

The methods of the present invention are useful for the treatment and prophylaxis of human subjects, particularly, infants, nursing mothers, surgical patients, individuals with foreign implanted medical devices or parts (e.g., catheters, prostheses, artificial hips, knees or limbs, dialysis access grafts, pacemakers and implantable defibrillators), patients with fistulas, immunocompromised patients, such as chemotherapy patients or patients taking steroids or immunosuppressive drugs (e.g., transplant patients, cancer patients and HIV positive individuals), patients with chronic illnesses, patients being cared for in health care facilities (e.g., hospitals, nursing homes, or dialysis centers) and patients who previously suffered from S. aureus infection, a Streptococcus infection, E. coli infection, P. aeruginosa infection, A. baumannii infection, E. faecium infection and/or C. difficile infection.

In some embodiments, the human subjects may be healthy individuals. In some embodiments, the human subjects may suffer, be suspected of suffering or be at risk of suffering from bacteremia, such as S. aureus bacteremia, a Streptococcus bacteremia, E. coli bacteremia, P. aeruginosa bacteremia, A. baumannii bacteremia, E. faecium bacteremia and/or C. difficile bacteremia. In addition to bacteremia, S. aureus infection may cause a broad range of illnesses from minor skin infections, such as atopic dermatitis, impetigo, boils, cellulitis, folliculitis, furuncles, carbuncles, scalded skin syndrome and abscesses, to life-threatening diseases such as staphylococcal pneumonia, staphylococcal meningitis, osteomyelitis, endocarditis, staphylococcal toxic shock syndrome (TSS) and septicemia. A Streptococcus infection may cause streptococcal pneumonia, streptococcal meningitis, streptococcal pharyngitis (“strep throat”), otitis media, scarlet fever, acute rheumatic fever, cellulitis, endocarditis, streptococcal TSS and perinatal Group B streptococcal disease. An E. coli infection may cause gastroenteritis, a urinary tract infection, neonatal meningitis, hemolytic-uremic syndrome (HUS), peritonitis, mastitis, septicemia and Gram-negative pneumonia. A P. aeruginosa infection may cause pneumonia, bacteremia, septicemia, a urinary tract infection, a gastrointestinal infection, ear and eye infections, a chronic lung infection, endocarditis, dermatitis and osteochondritis. An A. baumannii infection may cause nosocomial pneumonia and various other infections, such as skin and wound infections, bacteremia and meningitis. An E. faecium infection may cause urinary tract infections, bacteremia, bacterial endocarditis, diverticulitis and meningitis. A C. difficile infection is a common cause of colitis and pseudomembranous colitis in patients receiving antibiotic treatments for extended periods of time. In addition to colitis and pseudomembranous colitis, a C. difficile infection may cause severe diarrhea, toxic megacolon, intestinal perforation and even death. Accordingly, the methods of the present invention are useful for treating and preventing any of the above diseases associated with S. aureus, Streptococcus, E. coli, P. aeruginosa, A. baumannii, E. faecium and/or C. difficile infections.

In some embodiments, the S. aureus infection may be caused by a S. aureus strain that is resistant to an antibiotic, such as a methicillin-resistant strain (MRSA), a vancomycin intermediate strain (VISA) or vancomycin resistant strain (VRSA). In some embodiments, the S. aureus may be selected from methicillin resistant strains USA300 (also known as FPR 3757; ATCC #BAA-1556) and NYBK2464 (ATCC #BAA-51). In some embodiments, the Streptococcus infection may be caused by a S. pneumoniae strain that is resistant to an antibiotic, such as penicillin, tetracycline, clindamycin, a cephalosporin, a macrolide or a quinolone. In some embodiments, the E. coli infection may be caused by an E. coli strain that is resistant to an antibiotic, such as penicillin, streptomycin, chloramphenicol, ampicillin, cephalosporin or tetracycline. In some embodiments, the P. aeruginosa infection may be caused by a P. aeruginosa strain that is resistant to an antibiotic, such as a beta-lactams antibiotic (e.g., penicillin), piperacillin, imipenem, tobramycin or ciprofloxacin. In some embodiments, the A. baumannii infection may be caused by an A. baumannii strain that is resistant to an antibiotic, such as ceftazidime, gentamicin, ticarcillin, piperacillin, aztreonam, cefepime, ciprofloxacin, imipenem or meropenem. In some embodiments, the E. faecium infection may be caused by an E. faecium strain that is resistant to an antibiotic, such as penicillin, a cephalosporin or an aminoglycoside.

It is known in the art that certain bacterial antigens are conserved among different bacterial species and genera. Accordingly, the affinity purified human polyclonal antibodies of the present invention may also be useful for treating those humans who may be suffering, be suspected of suffering or be at risk of suffering from an additional bacterial infection. In some embodiments, the additional bacterial infection may be a Bacillus infection (e.g., B. anthracia), a Campylobacter infection (e.g., C. jejuni), a Clostridium infection (e.g., C. botulinum, C. perfringens, C. tetani), an Enterococcus infection (e.g., E. faecalis), a Helibacter infection (e.g., H. pylori), a Listeria infection (e.g., L. monocytogenes), a Mycobacterium infection (e.g., M. leprae, M. tuberculosis), a Salmonella infection (e.g., S. enterica) or a Shigella infection (e.g., S. flexneri, S. sonnei, S. dysenteriae).

The above therapeutic and prophylactic approaches may be combined with any one of a wide variety of therapeutic regimens for the treatment or prevention of bacterial infections. For example, the affinity purified human polyclonal antibodies of the present invention may be administered in conjunction with an additional therapeutic or preventive agent. The additional therapeutic or preventive agent may be an antibiotic, such as penicillin, a penicillinase-resistant penicillin (e.g., methicillin, oxacillin, cloxacillin, dicloxacillin or flucloxacillin), a glycopeptide (e.g., vancomycin) or an aminoglycoside (e.g., kanamycin, gentamicin or streptomycin), an antimicrobial agent, a bactericidal agent (e.g., lysostaphin), a bacteriostatic agent, or an immunostimulatory compound, such as a beta-glucan or GM-CSF.

### III. Antigenic Preparation

As discussed above, the antigenic preparations of the present invention comprise both secreted and/or cellular antigens of S. aureus, a Streptococcus, E. coli, P. aeruginosa, A. baumannii, E. faecium and/or C. difficile cells. In some embodiments, the antigenic preparations may comprise S. aureus, a Streptococcus, E. coli, P. aeruginosa, A. baumannii, E. faecium and/or C. difficile antigens comprising a peptide, a protein, a polynucleotide, a nucleic acid, a vitamin, a polysaccharide, a carbohydrate, a lipid and/or a complex thereof. In other embodiments, the lipid or the lipid component in the complex may be substantially removed in the antigenic preparation. In some embodiments, the polysaccharide, carbohydrate, or the polysaccharide or carbohydrate component in the complex may be substantially removed in the antigenic preparation. In other embodiments, S. aureus Protein A is substantially removed in the antigenic preparation in order to eliminate or substantially reduce the recovery of human antibodies that specifically bind to non-bacterial antigens. S. aureus Protein A may be substantially removed from the antigenic preparation by any suitable methods, e.g., by running the preparation through a chromatography column packed with cyanogen bromide (CNBR)-activated Sepharose 4B coupled to purified or highly purified human gamma globulin or human gamma globulin Fc fragments and collecting the eluate.

In some embodiments, the antigenic preparations may comprise a S. aureus capsular polysaccharide antigen, such as Type 5 antigen and Type 8 antigen. In other embodiments, the antigenic preparations may also comprise the S. aureus 336 antigen. In some embodiments, the antigenic preparations may comprise S. aureus toxins, such as pyrogenic toxin superantigens, exfoliative toxins and/or Staphylococcal toxins. Pyrogenic toxin superantigens (PTSAgs) have superantigen activities that induce toxic shock syndrome (TSS). This group includes the toxin TSST-1, which causes TSS associated with tampon use, and staphylococcal enterotoxins, such as S. aureus enterotoxin A (SEA) and S. aureus enterotoxin B (SEB), which cause food poisoning. Exfoliative toxins are implicated in the disease staphylococcal scalded-skin syndrome (SSSS), which occurs most commonly in infants and young children. Staphylococcal toxins that act on cell membranes include alpha-toxin, beta-toxin, delta-toxin, and several bicomponent toxins. The bicomponent toxin Panton-Valentine leukocidin (PVL) is associated with severe necrotizing pneumonia in children. The genes encoding the components of PVL are encoded on a bacteriophage found in community-associated MRSA strains. In some embodiments, the antigenic preparations may further comprise staphyloxanthin, a carotenoid pigment that has an antioxidant action that helps S. aureus cells evade killing with reactive oxygen radicals used by the host immune system.

In some embodiments, the antigenic preparations may also comprise a S. aureus antigen that confers resistance to antibiotics, such as penicillin, methicillin, aminoglycosides and/or vancomycin. Staphylococcal resistance to penicillin is mediated by penicillinase (a form of β-lactamase) production: an enzyme which breaks down the β-lactam ring of the penicillin molecule. Penicillinase-resistant penicillins such as methicillin, oxacillin, cloxacillin, dicloxacillin and flucloxacillin are able to resist degradation by staphylococcal penicillinase. Resistance to methicillin is mediated via the mec operon, part of the staphylococcal cassette chromosome mec (SCCmec). Resistance is conferred by the mecA gene, which codes for an altered penicillin-binding protein (PBP2a or PBP2′) that has a lower affinity for binding β-lactams (penicillins, cephalosporins and carbapenems). Resistance to aminoglycosides, such as kanamycin, gentamicin and streptomycin, is mediated by aminoglycoside modifying enzymes, ribosomal mutations and active efflux of the drug out of the bacteria. Aminoglycoside modifying enzymes inactivate the aminoglycoside by covalently attaching a phosphate, nucleotide or acetyl moiety to either the amine and/or alcohol functionality of the antibiotic, thereby rendering the antibiotic ineffective. The best characterized S. aureus aminoglycoside modifying enzyme is aminoglycoside 4′-O-nucleotidyltransferase, encoded by the ant(4′)-Ia gene. Vancomycin resistance is mediated by acquisition of the vanA gene, which codes for an enzyme that produces an alternative peptidoglycan to which vancomycin will not bind.

In some embodiments, the antigenic preparations may comprise a combination of two or more different antigens selected from a S. aureus capsular polysaccharide antigen, a S. aureus toxin, staphyloxanthin, and a S. aureus antigen that confers antibiotic resistance. Alternatively, the antigenic preparations may comprise a combination of two or more different antigens selected from a S. aureus toxin, staphyloxanthin, and a S. aureus antigen that confers antibiotic resistance.

In some embodiments, the Streptococcus infection may be caused by Streptococcus pneumoniae (S. pneumoniae), a Group A Streptococcus (GAS), such as Streptococcus pyogenes (S. pyogenes) or a Group B Streptococcus (GBS), such as Streptococcus agalactiae (S. agalactiae). In another embodiment, the Streptococcus may be selected from S. pneumoniae, S. pyogenes and S. agalactiae. In a further embodiment, the Streptococcus may be selected from GAS strain ATCC #19615 and GBS strain ATCC #25663.

S. pneumoniae expresses a number of different virulence factors on its cell surface and inside the organism. These virulence factors contribute to some of the clinical manifestations during infection with S. pneumoniae. S. pneumoniae polysaccharide capsule prevents phagocytosis by host immune cells by inhibiting C3b opsonization of the bacterial cells. Pneumolysin (Ply) is a toxin that causes lysis of host cells and activates complement. Activation of autolysin (LytA) leads to bacterial lysis releasing its internal contents, e.g., pneumolysin. Choline binding protein A/Pneumococcal surface protein A (CbpA/PspA) is an adhesion protein that can interact with carbohydrates on the cell surface of pulmonary epithelial cells and can inhibit complement-mediated opsonization of pneumococci.

In some embodiments, the antigenic preparations of the present invention may comprise two or more S. pneumoniae virulence factors selected from S. pneumoniae capsular polysaccharide antigens, autolysin (LytA), choline binding protein A/pneumococcal surface protein A (CbpA/PspA) and S. pneumoniae toxins, such as pneumolysin.

S. pyogenes has several virulence factors that enable it to attach to host tissues, evade the immune response, and spread by penetrating host tissue layers. A polysaccharide capsule composed of hyaluronic acid surrounds the bacterium, protecting it from phagocytosis by neutrophils. In addition, the capsule and several factors embedded in the cell wall, including M protein, lipoteichoic acid, and fibronectin-binding protein (protein F) facilitate attachment to various host cells. The M protein also inhibits opsonization by the alternative complement pathway by binding to host complement regulators.

S. pyogenes also secretes a number of virulence factors into its host, such as streptolysins O and S, streptococcal pyrogenic exotoxins (Spe) A, B and C, streptokinase, hyaluronidase, streptodornase, C5a peptidase and streptococcal chemokine protease. Streptolysins O and S are toxins which provide the basis of the organism's hemolytic property. Streptolysin 0 is a potent toxin affecting many cell types including neutrophils, platelets, and sub-cellular organelles. It causes an immune response and detection of antibodies to it, antistreptolysin 0 (ASO), can be clinically used to confirm a recent infection. Streptococcal pyrogenic exotoxins (Spe) A, B and C are superantigens secreted by many strains of S. pyogenes. These pyrogenic exotoxins are responsible for the rash of scarlet fever and many of the symptoms of streptococcal toxic shock syndrome. Streptokinase enzymatically activates plasminogen, a proteolytic enzyme, into plasmin, which in turn digests fibrin and other proteins. Hyaluronidase is currently presumed to facilitate the spread of S. pyogenes through infected tissues by breaking down hyaluronic acid, an important component of connective tissue. S. pyogenes streptodornases (DNAses) A-D protect the bacteria from being trapped in neutrophil extracellular traps (NETs) by destroying the NET's DNA, which serves as a scaffold for neutrophil serine proteases. C5a peptidase cleaves the potent neutrophil chemotaxin C5a, which reduces the influx of neutrophils early in infection as the bacteria start colonizing the host's tissue. Streptococcal chemokine protease (ScpC) also prevents the migration of neutrophils by degrading the chemokine IL-8, which normally attracts neutrophils to the site of infection.

In some embodiments, the antigenic preparations of the present invention may comprise two or more S. pyogenes virulence factors selected from S. pyogenes capsular polysaccharide antigens, M protein, lipoteichoic acid (LTA), fibronectin-binding protein (protein F), streptokinase, hyaluronidase, streptodornases A-D, C5a peptidase and streptococcal chemokine protease (ScpC), S. pyogenes toxins, such as streptolysins O and S, and streptococcal pyrogenic exotoxins (Spe), such as SpeA, SpeB and SpeC.

S. agalactiae's antiphagocytic polysaccharide capsule is this bacterium's main virulence factor. However, S. agalactiae also utilizes a number of accessory virulence factors, such as hyaluronidase, C5a peptidase, alpha C protein, glyceraldehyde 3-phosphate dehydrogenase (GAPDH) and S. agalactiae toxins, such as β-hemolysin (cytolysin) and the CAMP factor (protein B). Thus, in some embodiments, the antigenic preparations of the present invention may comprise two or more S. agalactiae virulence factors selected from S. agalactiae capsular polysaccharide antigens, hyaluronidase, C5a peptidase, alpha C protein, GAPDH and S. agalactiae toxins, such as β-hemolysin (cytolysin) and the CAMP factor (protein B).

In some embodiments, the E. coli infection may be caused by E. coli selected from enterotoxigenic E. coli (ETEC), enteropathogenic E. coli (EPEC), enteroinvasive E. coli (EIEC), enterohemorrhagic E. coli (EHEC), enteroaggregative E. coli (EAggEC) and uropathogenic E. coli (UPEC).

Enterotoxigenic E. coli (ETEC) is a causative agent of feverless diarrhea in humans. ETEC uses fimbrial adhesins, projections from the bacterial cell surface, to bind enterocytes in the small intestine. ETEC can produce two proteinaceous enterotoxins: the larger of the two proteins, the heat-labile LT enterotoxin, is similar to cholera toxin in structure and function, while the smaller protein, the heat-stable ST enterotoxin, causes cyclic guanosine monophosphate (cGMP) accumulation in the target cells and a subsequent secretion of fluid and electrolytes into the intestinal lumen.

Enteropathogenic E. coli (EPEC) is another causative agent of diarrhea in humans. EPEC lacks ST and LT toxins and fimbriae, but utilizes another adhesin known as intimin to bind host intestinal cells. This virotype has an array of virulence factors that are similar to those found in Shigella, and may possess a Shiga-like toxin.

Enteroinvasive E. coli (EIEC) is found exclusively in humans and causes a syndrome that is identical to Shigellosis, with profuse diarrhea and high fever. EIEC is highly invasive, and utilizes adhesin proteins to bind to and enter intestinal cells. It does not secrete toxins, but severely damages the intestinal wall through mechanical cell destruction.

Enterohemorrhagic E. coli (EHEC) typically causes bloody diarrhea and no fever, but can also cause hemolytic-uremic syndrome and sudden kidney failure. The best known member of this virotype is Shiga toxin-producing E. coli (STEC) strain O157:H7 (ATCC #43895). It uses bacterial fimbriae for attachment, is moderately-invasive and possesses a phage-encoded Shiga-like toxin that can elicit an intense inflammatory response.

Enteroaggregative E. coli (EAggEC) is found exclusively in humans and cause watery diarrhea without fever. EAggEC is non-invasive and uses fimbriae to binds to the intestinal mucosa. It produces a hemolysin and an ST enterotoxin similar to that of ETEC.

Uropathogenic E. coli (UPEC) is responsible for the bulk of human urinary tract infections (UTI). UPEC utilizes P fimbriae (pyelonephritis-associated pili) to bind urinary tract endothelial cells and colonize the bladder. These adhesins specifically bind D-galactose-D-galactose moieties on the P blood group antigen of erythrocytes and uroepithelial cells. UPEC also produces alpha- and beta-hemolysins, which cause lysis of urinary tract cells. It also has the ability to form K antigen, a capsular polysaccharide that contributes to biofilm formation.

In some embodiments, the antigenic compositions of the present invention may comprise two or more E. coli virulence factors selected from E. coli capsular polysaccharide antigens, such as K antigen, enterotoxins, such as heat-labile LT enterotoxins and heat-stable ST enterotoxins, adhesins, such as fimbrial adhesins and intimin, hemolysins, such as alpha-hemolysin and beta-hemolysin, and Shiga toxins.

P. aeruginosa features a number of virulence factors involved in colonization, invasion, and toxicogenesis. Virulence factors involved in colonization include adhesins, such as fimbriae (N-methyl-phenylalanine pili), capsule polysaccharides (glycocalyx) and mucoid exopolysaccharides (alginate). Virulence factors involved in invasion include invasins, such as proteases (elastase and alkaline protease), hemolysins (phospholipase and lecithinase), cytotoxin (leukocidin), and diffusible pigments (pyochelin and pyocyanin). Finally, virulence factors involved in toxicogenesis include lipopolysaccharide endotoxin and extracellular toxins, such as exoenzyme S and exotoxin A. Exoenzyme S has the characteristic subunit structure of the A-component of a bacterial toxin, and it has ADP-ribosylating activity for a variety of eukaryotic proteins that is characteristic of many bacterial exotoxins. Exotoxin A causes the ADP ribosylation of eukaryotic elongation factor 2 resulting in inhibition of protein synthesis in the affected cell.

In some embodiments, the present antigenic preparations may comprise two or more P. aeruginosa virulence factors selected from adhesins, such as fimbrial adhesins, capsule polysaccharides and mucoid exopolysaccharides, invasins, such as an elastase, an alkaline protease, hemolysins, such as a phospholipase and a lecithinase, leukocidin, a diffusible pigment, such as pyochelin and pyocyanin, lipopolysaccharide endotoxin, and extracellular toxins, such as exoenzyme S and exotoxin A.

Relatively, little is known about the virulence, antibiotic resistance, or persistence strategies of A. baumannii. The pathogenic determinants that have been reported thus far for A. baumannii include lipopolysaccharide 0, capsular exopolysaccharide, a novel pilus assembly system involved in biofilm formation, an outer membrane protein (Omp38) that causes apoptosis in human epithelial cells, and a polycistronic siderophore-mediated iron-acquisition system conserved between A. baumannii and Vibrio anguillarum. These factors presumably constitute a small fraction of elements involved in A. baumannii pathogenesis.

In some embodiments, the present antigenic preparations may comprise two or more A. baumannii virulence factors selected from lipopolysaccharide 0, capsular exopolysaccharide, pilus assembly system, membrane protein Omp38, and proteins of the polycistronic siderophore-mediated iron-acquisition system.

A number of enterococcal virulence factors have been described. Among them, gelatinase (GelE), aggregation substance (AS), hemagglutinin and cytolysin have been studied most intensively. GelE is a secreted extracellular zinc metalloendopeptidase secreted that shares homologies with GelE of Bacillus species and P. aeruginosa elastase. GelE can hydrolyze gelatin, casein, hemoglobin, and other bioactive peptides, which suggests its potential role as a virulence factor in enterococci. AS is involved in the conjugative transfer of plasmids, which can be observed as a clumping reaction. It has been demonstrated to mediate adhesion to cultured renal cells, suggesting that it may be important in the pathogenesis of infection. In addition to AS, hemagglutinin also contributes to the attachment to host cells. Cytolysin is a bacterial toxin that is encoded by an operon consisting of 8 genes localized on a pheromone-responsive plasmid or chromosome. Cytolysin shows hemolytic and bactericidal activity against other Gram-positive bacteria.

In some embodiments, the present antigenic preparations may comprise two or more E. faecium virulence factors selected from gelatinase (GelE), aggregation substance (AS), hemagglutinin and cytolysin.

Pathogenic C. difficile strains produce a number of virulence factors. The best characterized are enterotoxin (toxin A) and cytotoxin (toxin B), both of which are responsible for the diarrhea and inflammation seen in infected patients. Another toxin, referred to as “binary toxin,” has also been described in the scientific literature, but its role in Clostridium pathogenesis is not yet understood.

In some embodiments, the present antigenic preparations may comprise two or more C. difficile virulence factors selected from an enterotoxin, such as toxin A, a cytotoxin, such as toxin B, and a binary toxin.

In some embodiments, the antigenic preparations of the present invention may comprise a whole cell extract and secreted antigens of S. aureus, Streptococcus, E. coli, P. aeruginosa, A. baumannii, E. faecium and/or C. difficile. Such antigenic preparations may be obtained by any suitable methods, e.g., the method described below.

In some embodiments, the method comprising growing up bacterial cells, transferring them to a protein free media, and then growing the bacterial cells to secrete the toxins. Then without separating the cells from the toxins, the bacterial cells are disrupted in the same media that contain the bacterial toxins to obtain the antigenic preparations comprising a whole cell extract and secreted toxins. In other embodiments, the bacterial cells are separated from the toxins. The bacterial cells are collected, and disrupted to obtain a whole cell extract. The toxins are separately collected. The whole cell extract and the collected toxins are then combined to form the desired antigenic preparations.

In one embodiment, first, bacterial cells are grown in a protein containing culture medium for a specified period of time to reach a desired density. Second, the bacterial cells are collected (e.g., by filtration or centrifugation at 3,000 rpm for 15 minutes at 2-8° C.), resuspended in a non-protein containing culture medium and grown for another specified period of time in order to give the cells enough time to produce and secrete antigens (e.g., exotoxins) into the non-protein containing culture medium. Since S. aureus, Streptococcus, E. coli, P. aeruginosa, A. baumannii, E. faecium and C. difficile cells typically have different nutritional requirements and different growth rates, these growth steps are usually performed separately for each bacterial strain used. In some embodiments, different bacterial strains may be grown together in the same culture media if their nutritional requirements and growth conditions are sufficiently similar to permit joint culture.

Any suitable protein containing culture medium may be used to grow S. aureus cells. In some embodiments, the S. aureus protein containing culture medium may comprise the following ingredients: 17.0 g/L pancreatic digest of casein; 3.0 g/L enzymatic digest of soybean meal, 5.0 g/L NaCl, 2.5 g/L K₂HPO₄ and 2.5 g/L dextrose (Bacto™ Tryptic Soy Broth, 30% w/v in de-ionized H₂O; VWR Cat. No. 90000-378; Becton Dickinson Cat. No. 211825). Typically, S. aureus cells are grown in a protein containing culture medium for about 10 hours to about 72 hours at an appropriate temperature, e.g., 37° C. under conditions (for example mixing) familiar to those of skill in the art, to reach a density from 1×10⁹ to about 2×10⁹ before the cell collecting, e.g., pelleting, step. In some embodiments, the S. aureus non-protein containing culture medium may comprise an aqueous solution comprising sodium chloride, sodium phosphate, and optionally comprising a source of carbon, such as glucose or succinate. Typically, S. aureus cells are grown in a non-protein containing culture medium for about 10 hours to about 48 hours at an appropriate temperature, e.g., 37° C. under conditions (for example mixing) familiar to those of skill in the art.

Any suitable protein containing culture medium may be used to grow Streptococcus cells. In some embodiments, the Streptococcus protein containing culture medium may comprise the following ingredients: 17.0 g/L pancreatic digest of casein; 3.0 g/L enzymatic digest of soybean meal, 5.0 g/L NaCl, 2.5 g/L K₂HPO₄ and 2.5 g/L dextrose (Bacto™ Tryptic Soy Broth, 30% w/v in de-ionized H₂O; VWR Cat. No. 90000-378; Becton Dickinson Cat. No. 211825). Typically, Streptococcus cells are grown in a protein containing culture medium for about 10 hours to about 72 hours at an appropriate temperature, e.g., 37° C. under conditions (for example mixing) familiar to those of skill in the art to reach a density from 1×10⁹ to about 2×10⁹ before the cell collecting, e.g., pelleting, step. In some embodiments, the Streptococcus non-protein containing culture medium may comprise an aqueous solution comprising sodium chloride, sodium phosphate, and optionally comprising a source of carbon, such as glucose or succinate. Typically, Streptococcus cells are grown in a non-protein containing culture medium for about 10 hours to about 48 hours at an appropriate temperature, e.g., 37° C. under conditions (for example mixing) familiar to those of skill in the art.

Any suitable protein containing culture medium may be used to grow E. coli cells. In some embodiments, the E. coli protein containing culture medium may comprise the following ingredients: 17.0 g/L pancreatic digest of casein; 3.0 g/L enzymatic digest of soybean meal, 5.0 g/L NaCl, 2.5 g/L K₂HPO₄ and 2.5 g/L dextrose (Bacto™ Tryptic Soy Broth, 30% w/v in de-ionized H₂O; VWR Cat. No. 90000-378; Becton Dickinson Cat. No. 211825). Typically, E. coli cells are grown in a protein containing culture medium for about 10 hours to about 72 hours at an appropriate temperature, e.g., 37° C. under conditions (for example mixing) familiar to those of skill in the art to reach a density from 1×10⁹ to about 2×10⁹ before the cell collecting, e.g., pelleting, step. In some embodiments, the E. coli non-protein containing culture medium may comprise an aqueous solution comprising sodium chloride, sodium phosphate, and optionally comprising a source of carbon, such as glucose or succinate. Typically, E. coli cells are grown in a non-protein containing culture medium for about 10 hours to about 48 hours at an appropriate temperature, e.g., 37° C. under conditions (for example mixing) familiar to those of skill in the art.

Any suitable protein containing culture medium may be used to grow P. aeruginosa cells. In some embodiments, the P. aeruginosa protein containing culture medium may comprise the following ingredients: 17.0 g/L pancreatic digest of casein; 3.0 g/L enzymatic digest of soybean meal, 5.0 g/L NaCl, 2.5 g/L K₂HPO₄ and 2.5 g/L dextrose (Bacto™ Tryptic Soy Broth, 30% w/v in de-ionized H₂O; VWR Cat. No. 90000-378; Becton Dickinson Cat. No. 211825). Typically, P. aeruginosa cells are grown in a protein containing culture medium for about 10 hours to about 72 hours at an appropriate temperature, e.g., 37° C. under conditions (for example mixing) familiar to those of skill in the art to reach a density from 1×10⁹ to about 2×10⁹ before the cell collecting, e.g., pelleting, step. In some embodiments, the P. aeruginosa non-protein containing culture medium may comprise an aqueous solution comprising sodium chloride, sodium phosphate, and optionally comprising a source of carbon, such as glucose or succinate. Typically, P. aeruginosa cells are grown in a non-protein containing culture medium for about 10 hours to about 48 hours at an appropriate temperature, e.g., 37° C. under conditions (for example mixing) familiar to those of skill in the art.

Any suitable protein containing culture medium may be used to grow A. baumannii cells. In some embodiments, the A. baumannii protein containing culture medium may comprise the following ingredients: 17.0 g/L pancreatic digest of casein; 3.0 g/L enzymatic digest of soybean meal, 5.0 g/L NaCl, 2.5 g/L K₂HPO₄ and 2.5 g/L dextrose (Bacto™ Tryptic Soy Broth, 30% w/v in de-ionized H₂O; VWR Cat. No. 90000-378; Becton Dickinson Cat. No. 211825). Typically, A. baumannii cells are grown in a protein containing culture medium for about 10 hours to about 72 hours, preferably for about 48 hours, at an appropriate temperature, e.g., 37° C. under conditions (for example mixing) familiar to those of skill in the art to reach a density from 1×10⁹ to about 2×10⁹ before the cell collecting, e.g., pelleting, step. In some embodiments, the A. baumannii non-protein containing culture medium may comprise an aqueous solution comprising sodium chloride, sodium phosphate, and optionally comprising a source of carbon, such as glucose or succinate. Typically, A. baumannii cells are grown in a non-protein containing culture medium for about 10 hours to about 48 hours at an appropriate temperature, e.g., 37° C. under conditions (for example mixing) familiar to those of skill in the art.

Any suitable protein containing culture medium may be used to grow E. faecium cells. In some embodiments, the E. faecium protein containing culture medium may comprise the following ingredients: 17.0 g/L pancreatic digest of casein; 3.0 g/L enzymatic digest of soybean meal, 5.0 g/L NaCl, 2.5 g/L K₂HPO₄ and 2.5 g/L dextrose (Bacto™ Tryptic Soy Broth, 30% w/v in de-ionized H₂O; VWR Cat. No. 90000-378; Becton Dickinson Cat. No. 211825). Typically, E. faecium cells are grown in a protein containing culture medium for about 10 hours to about 72 hours at an appropriate temperature, e.g., 37° C. under conditions (for example mixing) familiar to those of skill in the art, to reach a density from 1×10⁹ to about 2×10⁹ before the cell collecting, e.g., pelleting, step. In some embodiments, the E. faecium non-protein containing culture medium may comprise an aqueous solution comprising sodium chloride, sodium phosphate, and optionally comprising a source of carbon, such as glucose or succinate. Typically, E. faecium cells are grown in a non-protein containing culture medium for about 10 hours to about 48 hours at an appropriate temperature, e.g., 37° C. under conditions (for example mixing) familiar to those of skill in the art.

Any suitable protein containing culture medium may be used to grow C. difficile cells. In some embodiments, the C. difficile protein containing culture medium may comprise the following ingredients: 5.0 g/L pancreatic digest of casein, 5.0 g/L proteose peptone #3, 10.0 g/L beef extract, 3.0 g/L yeast extract, 5.0 g/L NaCl, 1.0 g/L soluble starch, 5.0 g/L dextrose, 0.5 g/L cysteine HCl and 3.0 g/L sodium acetate (Difco™ Reinforced Clostridial Media, 38% w/v in de-ionized H₂O; Becton Dickinson Cat. No. 218081). Typically, C. difficile cells are grown in a protein containing culture medium for about 10 hours to about 72 hours at an appropriate temperature, e.g., 37° C. under conditions (for example mixing) familiar to those of skill in the art to reach a density from 1×10⁹ to about 2×10⁹ before the cell collecting, e.g., pelleting, step. In some embodiments, the C. difficile non-protein containing culture medium may comprise an aqueous solution comprising sodium chloride, sodium phosphate, and optionally comprising a source of carbon, such as glucose or succinate. Typically, C. difficile cells are grown in a non-protein containing culture medium for about 10 hours to about 48 hours at an appropriate temperature, e.g., 37° C. under conditions (for example mixing) familiar to those of skill in the art.

In the next step, the bacterial cells are harvested by centrifugation at 20,000 rpm for 15-30 minutes at 2-8° C., resuspended in 10 volumes of sterile phosphate buffered saline (PBS), pH 7.5, and pelleted by another centrifugation at 20,000 rpm for 15-30 minutes at 2-8° C. The wash procedure is repeated two more times in order to completely remove the culture medium. The bacterial cells can be disrupted by any suitable methods. In some embodiments, the bacterial cells are disrupted with a Microfluidizer® high-shear fluid processor (Microfluidics Corp., Newton, Mass.) twice under 20,000 psi at 150 ml/min Disruption of the bacterial cells can also be accomplished by homogenization (e.g., by using the Potter-Elvehjem homogenizer, Dounce homogenizer, or French press), freeze thaw and/or sonication, after which insoluble cellular debris (e.g., bacterial walls and nuclei) are removed, e.g., filtered or pelleted (e.g., by centrifugation at 4,000 rpm for 30 minutes at 2-8° C.), and the supernatant containing cellular antigens is collected. In some embodiments, detergent cell lysis may be used alone or in conjunction with homogenization, freeze thaw and/or sonication to disrupt the bacterial cells. The choice of detergent depends on the cells to be disrupted, particularly on the presence or absence of a bacterial cell wall. In general, non-ionic (e.g., Triton-X®) and zwitterionic (e.g., CHAPS) detergents are milder and less denaturing than ionic detergents. In contrast, ionic detergents (e.g., SDS) are strong solubilizing agents and tend to denature proteins, thereby destroying protein activity and function. There are also ionic detergents that are only mildly denaturing (e.g., sodium cholate and sodium deoxycholate). In some embodiments, it may be preferable to use a dialyzable detergent to facilitate its removal from the lysis solution.

Antigens secreted by the bacterial cells into the non-protein containing culture medium are also collected. In some embodiments, the secreted antigens are collected separately from the whole cell extract by precipitating the bacterial cells prior to the bacterial cell disruption step and by collecting the supernatant. In other embodiments, the disruption step is carried out in the presence of the secreted antigens, so that the secreted antigens are combined with the cellular antigens immediately upon the disruption of the bacterial cells.

In some embodiments, the disruption and collection steps are performed separately for each bacterial strain. In other embodiments, two or more bacterial strains are combined prior to the disruption and collection of the cellular and secreted antigens. In some embodiments, the S. aureus, Streptococcus, E. coli, P. aeruginosa, A. baumannii, E. faecium and C. difficile cultures are pooled prior to the bacterial cell disruption and the collection of the secreted and cellular antigens.

In some embodiments, optimal bacterial cell lysis conditions are used to maximize the amount of extracted protein while minimizing protein oxidation, unwanted proteolysis and sample contamination with genomic DNA. See e.g., Protein production and purification, Nature Methods, 5(2):135-146 (2008). Mechanical lysis by high-pressure homogenization or sonication, or lysis by freeze-thaw procedures with lysozyme are equivalent in most cases. The lysis buffer may contain a strong buffer (e.g., 50-100 mM phosphate or HEPES) to overcome the contribution of the bacterial lysate, high ionic strength (e.g., equivalent to 300-500 mM NaCl) to enhance protein solubility and stability, protease inhibitors and a reducing agent such as dithiothreitol (DTT) or Tris(2-carboxyethyl) phosphine hydrochloride (TCEP) to prevent oxidation of the protein. Inclusion of glycerol (10%) during protein purification enhances the solubility and stability of many proteins. Loading large amounts of bacterial lysate (e.g., >1 L culture volume) on relatively small (e.g., <1 ml) affinity columns may require prior removal of any particulate or viscous material. This can be accomplished by using enzymes that degrade nucleic acid and cell-wall material, such as DNase or Benzonase (Merck/EMD) and lysozyme, respectively. Some of the enzymes used in lysis are less active in the presence of reducing agents or high salt concentration; optimal lysis may require sequential addition of the components. Clarified lysates can also be filtered before loading on the affinity columns.

A wide variety of bacterial lysis solutions that are suitable for total protein extraction are currently available. By way of illustration and not limitation, suitable bacterial lysis compositions may include: 20 mM HEPES, pH 7.6, 500 mM NaCl, 1 mM EDTA, 10% (v/v) glycerol, 1 mM PMSF, 5 μg/ml leupeptine, 1% (v/v) aprotinin and 0.1% NP-40; 10 mM Tris-HCl, pH 7.4, 1 mM EDTA, 8 M Urea, 50 mM DTT, 10% (v/v) glycerol, 5% v/v NP-40 and 6% (w/v) ampholytes (i.e., amphoteric compounds containing both acidic and basic groups); CelLytic™ B, CelLytic™ B-II and CelLytic™ B Plus Protein Extraction Reagents (Sigma-Aldrich, Part Nos. B3553, B3678 and CB0500); B-PER® Bacterial Protein Extraction Reagent (Pierce Biotechnology, Part No. 78248); EasyLyse™ Bacterial Protein Extraction Solution (Epicentre Biotechnologies, Part No. RP03750); or Easy BacLysis Protein Extraction Solution (GenScript, Part Nos. L00230 and L00240).

The secreted antigens of S. aureus, a Streptococcus, E. coli, P. aeruginosa, A. baumannii, E. faecium and C. difficile may comprise well-characterized exotoxins that may be used as benchmarks for assessing the quality and/or concentration of the antigenic preparation.

In some embodiments, the secreted antigens of S. aureus may comprise staphylococcal enterotoxin A (SEA) and/or staphylococcal enterotoxin B (SEB). The SEA may be present in the secreted antigens at a concentration of about 0.01 μg/mL to about 400 μg/mL, preferably about 0.01 μg/mL to about 5 μg/mL, whereas the SEB may be present at a concentration of about 0.01 μg/mL to about 400 μg/mL, preferably about 10 μg/mL to about 400 μg/mL. Similarly, in some embodiments, the antigenic preparations may comprise a S. aureus whole cell extract and SEA and/or SEB. The SEA may be present in the antigenic preparations at a concentration of about 0.01 μg/mL to about 400 μg/mL, preferably about 0.01 μg/mL to about 5 μg/mL, whereas the SEB may be present at a concentration of about 0.01 μg/mL to about 400 μg/mL, preferably about 10 μg/mL to about 400 μg/mL.

In some embodiments, the secreted antigens of Streptococcus may comprise Streptococcal pyrogenic exotoxin A (SpeA) and/or Streptococcal pyrogenic exotoxin C (SpeC). The SpeA may be present in the secreted antigens at a concentration of about 0.01 μg/mL to about 400 μg/mL, preferably about 5 μg/mL to about 20 μg/mL, whereas the SpeC may be present at a concentration of about 0.01 μg/mL to about 400 μg/mL, preferably about 0.01 μg/mL to about 10 μg/mL. Similarly, in some embodiments, the antigenic preparations may comprise a Streptococcus whole cell extract and SpeA and/or SpeC. The SpeA may be present in the antigenic preparations at a concentration of about 0.01 μg/mL to about 400 μg/mL, preferably about 5 μg/mL to about 20 μg/mL, whereas the SpeC may be present at a concentration of about 0.01 μg/mL to about 400 μg/mL, preferably about 0.01 μg/mL to about 10 μg/mL.

In some embodiments, the secreted antigens of E. coli may comprise a Shiga-like toxin. The Shiga-like toxin may be present in the secreted antigens at a concentration of about 0.01 μg/mL to about 400 μg/mL, preferably about 0.25 μg/mL to about 4 μg/mL. Similarly, in some embodiments, the antigenic preparations may comprise an E. coli whole cell extract and a Shiga-like toxin. The Shiga-like toxin may be present in the antigenic preparations at a concentration of about 0.01 μg/mL to about 400 μg/mL, preferably about 0.25 μg/mL to about 4 μg/mL.

In some embodiments, the secreted antigens of P. aeruginosa may comprise exoenzyme S (PES) and/or exotoxin A (PEA). The PES may be present in the secreted antigens at a concentration of about 0.01 μg/mL to about 400 μg/mL, whereas the PEA may be present at a concentration of about 0.01 μg/mL to about 400 μg/mL. Similarly, in some embodiments, the antigenic preparations may comprise a P. aeruginosa whole cell extract and PES and/or PEA. The PES may be present in the antigenic preparations at a concentration of about 0.01 μg/mL to about 400 μg/mL, whereas the PEA may be present at a concentration of about 0.01 μg/mL to about 400 μg/mL.

In some embodiments, the secreted antigens of C. difficile may comprise toxin A (CTA) and/or toxin B (CTB). The CTA may be present in the secreted antigens at a concentration of about 0.01 μg/mL to about 400 μg/mL, whereas the CTB may be present at a concentration of about 0.01 μg/mL to about 400 μg/mL. Similarly, in some embodiments, the antigenic preparations may comprise a C. difficile whole cell extract and CTA and/or CTB. The CTA may be present in the antigenic preparations at a concentration of about 0.01 μg/mL to about 400 μg/mL, whereas the CTB may be present at a concentration of about 0.01 μg/mL to about 400 μg/mL.

### IV. Affinity Purified Human Polyclonal Antibodies

In some embodiments, the starting material for the polyclonal antibodies of the present invention is a serum, plasma or whole blood sample. If a whole blood sample is used, it may be subjected to some preliminary processing steps such as dilution or removing particulate materials from the blood sample. In some embodiments, the blood sample is obtained from a normal human who is not hyperimmune to S. aureus, a Streptococcus, E. coli, P. aeruginosa, A. baumannii, E. faecium and/or C. difficile as a result of recent vaccination against S. aureus, a Streptococcus, E. coli, P. aeruginosa, A. baumannii, E. faecium and/or C. difficile or recent exposure to an acute S. aureus, Streptococcus, E. coli, P. aeruginosa, A. baumannii, E. faecium and/or C. difficile infection, especially the infection that led to bacteremia. In other embodiments, the blood sample is obtained from a human who is hyperimmune to S. aureus, a Streptococcus, E. coli, P. aeruginosa, A. baumannii, E. faecium and/or C. difficile as a result of recent vaccination against S. aureus, a Streptococcus, E. coli, P. aeruginosa, A. baumannii, E. faecium and/or C. difficile or recent exposure to an acute S. aureus, Streptococcus, E. coli, P. aeruginosa, A. baumannii, E. faecium and/or C. difficile infection, especially the infection that led to bacteremia. In some embodiments, the blood sample is human plasma from a normal human donor that has been lipid stripped with the use of fumed silica, dextran sulfate or other conventional processes such as using organic solvents capable of solubulizing lipids. In some embodiments, the blood sample is human gamma globulin (IgG) from a normal human donor prepared by known methods, such as cold alcohol Cohn fractionation, ammonium sulfate precipitation, caprylic acid precipitation and/or sodium sulfate precipitation.

For bacteria that are ubiquitous, the person who is not currently infected with the organism may be de facto hyperimmune as the person may have been exposed and is now protected from that organism. The advantage of using hyperimmune plasma is only one of quantity, i.e., there is a greater quantity of antibody in the plasma from the hyperimmune individual. Normal human plasma may have just as potent and therapeutically effective antibodies as the hyperimmune person; it is only present in lower concentrations. This disadvantage can be overcome with the use of much greater quantities of normal human plasma as compared to the quantity of hyperimmune plasma.

One important advantage of the present therapeutic and preventive methods is that they can be adapted to infectious serotypes typical of a particular geographic region by using locally collected and current human blood samples. Thus, in some embodiments, the human blood sample may be collected from a geographic area in which the anti-bacterial treatment is administered. In some embodiments, the human blood sample may be collected from a geographic area in which a recipient of the anti-bacterial treatment resides. Alternatively, the human blood sample may be collected from a geographic area to which a recipient of the anti-bacterial treatment intends to travel.

In some embodiments, the blood sample is pooled from at least 2 humans, preferably from at least 10 humans, more preferably from at least 100 humans and most preferably from at least 1000 humans. In other embodiments, the blood sample is pooled from at least 2 normal humans, preferably from at least 10 normal humans, more preferably from at least 100 normal humans and most preferably from at least 1000 normal humans.

The desired human polyclonal antibodies can be purified or affinity purified from a human blood sample by any suitable methods. In some embodiments, to purify the human blood sample for the desired human polyclonal antibodies, one first attaches one of the S. aureus, Streptococcus, E. coli, P. aeruginosa, A. baumannii, E. faecium and/or C. difficile antigenic preparations described above to cross linked agarose beads (e.g., cyanogen bromide (CNBr) activated Sepharose 4B from Pharmacia, Uppsala, Sweden), according to manufacturer's instructions. Prior to loading the antigenic preparation onto a chromatography column, CNBr-activated Sepharose 4B may be sterilized with 70% ethyl alcohol (pH 3.0) for about 30 minutes. Next, one uses these agarose beads with the antigenic preparation coupled to them to pack an affinity separation column. The column is then washed and equilibrated with a suitable wash buffer, e.g., 0.01 M phosphate buffered saline (PBS), pH 7.4. The human blood sample is loaded onto the column and washed with 0.01 M PBS in order to remove antibodies without the S. aureus, Streptococcus, E. coli, P. aeruginosa, A. baumannii, E. faecium and/or C. difficile antigen binding specificity. The bound human polyclonal antibodies specific to S. aureus, a Streptococcus, E. coli, P. aeruginosa, A. baumannii, E. faecium and/or C. difficile antigen are eluted from the solid phase antigenic preparation in the column by passing an elution solution, e.g., 0.1 M glycine hydrochloride buffer, pH 2.5-2.75 through the column. The eluted polyclonal antibodies are neutralized after they leave the column with either the addition of a neutralizing solution or buffer, e.g., 1 M phosphate buffer, pH 8 or by a buffer exchange with 0.01 M PBS, as is known to those of skill in the art. The eluate containing human polyclonal antibodies specific to S. aureus, a Streptococcus, E. coli, P. aeruginosa, A. baumannii, E. faecium and/or C. difficile antigen can optionally be concentrated and buffer exchanged into a solution for administering to a human, e.g., a sterile aqueous solution containing 10% maltose and 0.03 Polysorbate 80, pH 5.6. The solution can also be filtered to remove any residual particulate and stored at a suitable temperature, e.g., 2-8° C. In some embodiments, the preparation is purified to remove antibody aggregates in order to produce a monomeric antibody preparation.

It is noted that the same human blood sample may be subjected to multiple cycles of affinity purification using different antigenic preparations. For example, a human blood sample that has been depleted of anti-S. aureus polyclonal antibodies may be collected and subjected to a further round of affinity purification using a Streptococcus antigenic preparation, and so forth. Thus, the present invention contemplates both “parallel” affinity purification, wherein human polyclonal antibodies against multiple bacterial species are isolated simultaneously, and “serial” affinity purification, wherein human polyclonal antibodies against multiple bacterial species are isolated sequentially by reusing the same human blood sample in multiple cycles of affinity purification.

The affinity purified human polyclonal antibodies can have suitable concentrations for a desired purpose, e.g., storage or administration. In some embodiments, the affinity purified human polyclonal antibodies of the present invention have a concentration in the range between about 10 μg/ml and about 10 mg/ml, preferably between about 100 μg/ml and about 5 mg/ml, more preferably between about 300 μg/ml and about 3 mg/ml and most preferably about 2 mg/ml. In some embodiments, the affinity purified human polyclonal antibodies can have suitable concentrations at about 5 mg/ml, 10 mg/ml, 20 mg/ml, 30 mg/ml, 40 mg/ml, or 50 mg/ml.

In other embodiments, the affinity purified human polyclonal antibodies are purified from about 2 fold to about 50,000 fold, preferably at least 10 fold, more preferably at least 100 fold and most preferably at least 1,000 fold relative to the same human polyclonal antibodies in the human blood sample. In some embodiments, the affinity purified human polyclonal antibodies have an in vivo or in vitro antibacterial or antigen binding activity per milligram of protein that is about 2 to 50,000 times higher, preferably at least 10 times higher, more preferably at least 100 times higher and most preferably at least 1,000 times higher than the corresponding in vivo or in vitro antibacterial or antigen binding activity per milligram of unpurified human immunoglobulin, or non-affinity-purified human immunoglobulin sample, e.g., intravenous immunoglobulin (IVIG) sample.

In some embodiments, the affinity purified human polyclonal antibodies are substantially free of at least 10%, 20%, 30%, 40%, 50%, 60%, 70%, preferably at least 80%, more preferably at least 90% and most preferably at least 95% of human antibodies that specifically bind to non-bacterial antigens in the human blood sample.

### V. Immunological Testing

Another important aspect of the present invention concerns the use of affinity purified human polyclonal antibodies for identifying those individuals who may be suitable for polyclonal antibody therapy or prophylaxis of bacterial infection, for monitoring the progress and/or efficacy of the therapeutic or prophylactic treatment and for determining an optimal therapeutic or prophylactic dose based on an individual's initial response to the treatment with affinity purified human polyclonal antibodies.

In some embodiments, the therapeutic and preventive methods of the present invention comprise conducting an immunotest prior to administering the affinity purified human polyclonal antibodies against S. aureus, a Streptococcus, E. coli, P. aeruginosa, A. baumannii, E. faecium and/or C. difficile antigen to an individual, in order to assess the suitability of the individual for the therapeutic or preventive antibacterial antibody treatment. The same affinity purified human polyclonal antibodies against S. aureus, a Streptococcus, E. coli, P. aeruginosa, A. baumannii, E. faecium and/or C. difficile antigen are used to determine the presence, absence and/or amount of bacterial antigens in a suitable sample, e.g., a blood sample, from a candidate for the polyclonal antibody treatment. A positive immunotest result indicates that the candidate is suitable for therapy or prevention of bacterial infection using the affinity purified human polyclonal antibodies against S. aureus, a Streptococcus, E. coli, P. aeruginosa, A. baumannii, E. faecium and/or C. difficile infection.

In some embodiments, the therapeutic and preventive methods of the present invention comprise conducting an immunotest before and after administering the affinity purified human polyclonal antibodies against S. aureus, a Streptococcus, E. coli, P. aeruginosa, A. baumannii, E. faecium and/or C. difficile antigen to an individual, in order to monitor the efficacy of the therapeutic, removal or preventive treatment. The same affinity purified human polyclonal antibodies against S. aureus, a Streptococcus, E. coli, P. aeruginosa, A. baumannii, E. faecium and/or C. difficile antigen are used to determine the presence, absence and/or amount of bacterial antigens in a suitable sample, e.g., blood samples, taken from the treated individual before and after the administration of the antibodies. The absence or reduction in the bacterial antigens after administering the affinity purified human polyclonal antibodies to the individual relative to the amount of bacterial antigens before the treatment indicates efficacy of the therapeutic, removal or preventive treatment.

In some embodiments, the therapeutic, removal or preventive methods of the present invention comprise conducting an immunotest before and after administering the affinity purified human polyclonal antibodies against S. aureus, a Streptococcus, E. coli, P. aeruginosa, A. baumannii, E. faecium and/or C. difficile antigen to an individual, in order to determine an optimal therapeutic or prophylactic dose based on the individual's response to the treatment with affinity purified human polyclonal antibodies. The same affinity purified human polyclonal antibodies against S. aureus, a Streptococcus, E. coli, P. aeruginosa, A. baumannii, E. faecium and/or C. difficile antigen are used to determine the presence, absence and/or amount of bacterial antigens in a suitable sample, e.g., blood samples, taken from the treated individual before and after the administration of the antibodies. The optimal therapeutic, removal or prophylactic dose of the affinity purified human polyclonal antibodies is determined based on the amount of the bacterial antigens remaining after administering the affinity purified human polyclonal antibodies to the individual and the extent of reduction in the bacterial antigens after administering the affinity purified human polyclonal antibodies relative to the amount of bacterial antigens before the administration.

A variety of immunotests are contemplated. In some embodiments, the present methods assess the complex formed between bacterial antigens and affinity purified human polyclonal antibodies via a sandwich or competitive assay format. In other embodiments, the complex is assessed in a homogeneous or a heterogeneous assay format. In some embodiments, the complex is assessed by a format selected from the group consisting of an enzyme-linked immunosorbent assay (ELISA), chemiluminescent assay, immunoblotting, immunoprecipitation, radioimmunoassay (RIA), immunostaining, latex agglutination, indirect hemagglutination assay (IHA), complement fixation, indirect immunofluorescent assay (IFA), nephelometry, flow cytometry assay, plasmon resonance assay, chemiluminescence assay, lateral flow immunoassay, μ-capture assay, inhibition assay and avidity assay. In other embodiments, the immunotest is conducted as a precipitation or an agglutination assay.

### VI. Pharmaceutical Compositions and Formulations

In one aspect, the present invention concerns pharmaceutical compositions for treating or preventing bacterial infections, which comprise an effective amount of human polyclonal antibodies affinity purified from a human blood sample with an antigenic preparation comprising cellular and/or secreted antigen(s) from bacterial cells selected from the group consisting of S. aureus, a Streptococcus, E. coli, P. aeruginosa, A. baumannii, E. faecium, C. difficile and a combination thereof. Preferably, the affinity purified human polyclonal antibodies are purified (e.g., as made more concentrated as compared to the starting or unpurified material) relative to the same human polyclonal antibodies in the unpurified or non-affinity-purified human blood sample, e.g., intravenous immunoglobulin (IVIG) sample. Also preferably, the affinity purified human polyclonal antibodies are specific for the bacterial antigen(s) used in the affinity purification. Further preferably, the affinity purified human polyclonal antibodies are substantially free of human antibodies that specifically bind to non-bacterial antigens in the human blood sample.

In some embodiments, the antigenic preparations used to purify the polyclonal human antibodies may comprise cellular and/or secreted antigen(s) from S. aureus. In some embodiments, the antigenic preparations may comprise cellular and/or secreted antigen(s) from a Streptococcus. In some embodiments, the antigenic preparations may comprise cellular and/or secreted antigen(s) from E. coli. In some embodiments, the antigenic preparations may comprise cellular and/or secreted antigen(s) from P. aeruginosa. In some embodiments, the antigenic preparations may comprise cellular and/or secreted antigen(s) from A. baumannii. In some embodiments, the antigenic preparations may comprise cellular and/or secreted antigen(s) from E. faecium. In some embodiments, the antigenic preparations may comprise cellular and/or secreted antigen(s) from C. difficile.

In some embodiments, the antigenic preparations may comprise cellular and/or secreted antigens from a combination of any two bacterial species selected from S. aureus, a Streptococcus, E. coli, P. aeruginosa, A. baumannii, E. faecium and C. difficile. In some embodiments, the antigenic preparations may comprise cellular and/or secreted antigens from a combination of any three bacterial species selected from S. aureus, a Streptococcus, E. coli, P. aeruginosa, A. baumannii, E. faecium and C. difficile. In some embodiments, the antigenic preparations may comprise cellular and/or secreted antigens from a combination of any four bacterial species selected from S. aureus, a Streptococcus, E. coli, P. aeruginosa, A. baumannii, E. faecium and C. difficile. In some embodiments, the antigenic preparations may comprise cellular and/or secreted antigens from a combination of any five bacterial species selected from S. aureus, a Streptococcus, E. coli, P. aeruginosa, A. baumannii, E. faecium and C. difficile. In some embodiments, the antigenic preparations may comprise cellular and/or secreted antigens from a combination of any six bacterial species selected from S. aureus, a Streptococcus, E. coli, P. aeruginosa, A. baumannii, E. faecium and C. difficile. Alternatively, the antigenic preparations may comprise cellular and/or secreted antigens from each of S. aureus, a Streptococcus, E. coli, P. aeruginosa, A. baumannii, E. faecium and C. difficile. In some embodiments, the antigenic preparations comprise cellular and/or secreted antigens from each of S. aureus, S. pyogenes, S. pneumoniae, E. coli, P. aeruginosa, A. baumannii, E. faecium and C. difficile.

In another aspect, the present invention concerns pharmaceutical compositions for treating or preventing bacterial infections, which comprise an effective amount of human polyclonal antibodies affinity purified from a human blood sample with an antigenic preparation comprising cellular and/or secreted antigens from two or more different bacterial species selected from the group consisting of S. aureus, a Streptococcus, E. coli, P. aeruginosa, A. baumannii, E. faecium and C. difficile. Preferably, the affinity purified human polyclonal antibodies are purified (e.g., as made more concentrated as compared to the starting or unpurified material) relative to the same human polyclonal antibodies in the unpurified or non-affinity-purified human blood sample, e.g., intravenous immunoglobulin (IVIG) sample. Also preferably, the affinity purified human polyclonal antibodies are specific for the bacterial antigen(s) used in the affinity purification. Further preferably, the affinity purified human polyclonal antibodies are substantially free of human antibodies that specifically bind to non-bacterial antigens in the human blood sample.

In some embodiments, the antigenic preparations may comprise cellular and/or secreted antigens from a combination of any two bacterial species selected from S. aureus, a Streptococcus, E. coli, P. aeruginosa, A. baumannii, E. faecium and C. difficile. For example, the antigenic preparation may comprise a secreted antigen from one bacterial species and a cellular antigen from another bacterial species, or secreted antigens from two different bacterial species, or cellular antigens from two different bacterial species. In some embodiments, the antigenic preparations may comprise cellular and/or secreted antigens from a combination of any three bacterial species selected from S. aureus, a Streptococcus, E. coli, P. aeruginosa, A. baumannii, E. faecium and C. difficile. In some embodiments, the antigenic preparations may comprise cellular and/or secreted antigens from a combination of any four bacterial species selected from S. aureus, a Streptococcus, E. coli, P. aeruginosa, A. baumannii, E. faecium and C. difficile. In some embodiments, the antigenic preparations may comprise cellular and/or secreted antigens from a combination of any five bacterial species selected from S. aureus, a Streptococcus, E. coli, P. aeruginosa, A. baumannii, E. faecium and C. difficile. In some embodiments, the antigenic preparations may comprise cellular and/or secreted antigens from a combination of any six bacterial species selected from S. aureus, a Streptococcus, E. coli, P. aeruginosa, A. baumannii, E. faecium and C. difficile. Alternatively, the antigenic preparations may comprise cellular and/or secreted antigens from each of S. aureus, a Streptococcus, E. coli, P. aeruginosa, A. baumannii, E. faecium and C. difficile. In some embodiments, the antigenic preparations comprise cellular and/or secreted antigens from each of S. aureus, S. pyogenes, S. pneumoniae, E. coli, P. aeruginosa, A. baumannii, E. faecium and C. difficile.

The pharmaceutical compositions may also comprise an additional therapeutic or preventive agent. The additional therapeutic or preventive agent may be an antibiotic, such as penicillin, a penicillinase resistant penicillin (e.g., methicillin, oxacillin, cloxacillin, dicloxacillin or flucloxacillin), a glycopeptide (e.g., vancomycin) or an aminoglycoside (e.g., kanamycin, gentamicin or streptomycin), an antimicrobial agent, a bactericidal agent (e.g., lysostaphin), a bacteriostatic agent, or an immunostimulatory compound, such as a beta-glucan or GM-CSF.

The affinity purified human polyclonal antibodies can be incorporated into a wide variety of pharmaceutical compositions suitable for administration. Such compositions typically comprise the agent and a pharmaceutically acceptable carrier or excipient. Supplementary active compounds can also be incorporated into the compositions. Various pharmaceutical compositions and techniques for their preparation and use will be known to those of skill in the art in light of the present disclosure. For a detailed listing of suitable pharmacological compositions and associated administrative techniques one may refer to the detailed teachings herein, which may be further supplemented by texts such as REMINGTON: THE SCIENCE AND PRACTICE OF PHARMACY, 20th Ed. (Lippincott, Williams & Wilkins 2003).

Exemplary formulations include, but are not limited to, those suitable for parenteral administration, e.g., intravenous, intra-arterial, intramuscular, or subcutaneous administration, including formulations encapsulated in micelles, liposomes or drug-release capsules (active agents incorporated within a biocompatible coating designed for slow-release); ingestible formulations; formulations for topical use, such as creams, ointments and gels; and other formulations such as inhalants, aerosols and sprays. Further, those of ordinary skill in the art can readily deduce that suitable formulations involving these compositions and dosage forms, including those formulations as described elsewhere herein.

Pharmaceutically-acceptable materials, composition or vehicle, such as a liquid or solid filler, diluent, excipient, solvent or encapsulating material, involved in carrying or transporting the subject moiety or chemical, e.g., an antibody, from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Therapeutic formulations can be solubilized and administered via any route capable of delivering the therapeutic composition to the subject. One exemplary formulation for intravenous injection comprises the therapeutic antibody composition in an aqueous solution comprising bacteriostatic or sterile water, 10% maltose and 0.03% Polysorbate 80, pH 5.5. Another formulation for intravenous injection comprises the therapeutic antibody composition in an aqueous solution comprising bacteriostatic or sterile water and about 0.2 M glycine, pH 4.0-4.5. Therapeutic preparations can be lyophilized and stored as sterile powders, preferably under vacuum, and then reconstituted in bacteriostatic water or in sterile water prior to injection.

In a further aspect, the present invention also provides methods for treating or preventing a bacterial infection, which comprise administering to a human suffering, suspected of suffering or at risk of suffering from Staphylococcus aureus (S. aureus) infection, a Streptococcus infection, Escherichia coli (E. coli) infection, Pseudomonas aeruginosa (P. aeruginosa) infection, Acinetobacter baumannii (A. baumannii) infection, Enterococcus faecium (E. faecium) infection and/or Clostridium difficile (C. difficile) infection an effective amount of any of the above pharmaceutical compositions comprising affinity purified human polyclonal antibodies against S. aureus, a Streptococcus, E. coli, P. aeruginosa, A. baumannii, E. faecium and/or C. difficile.

### VII. Administration and Dosage

Single or multiple doses of the affinity purified human polyclonal antibodies may be delivered to a human subject using any convenient mode of administration, including but not limited to intravenous, intraperitoneal, intracorporeal, intra-articular, intraventricular, intrathecal, intramuscular, subcutaneous, intranasal, intravaginal, topical and oral administration. In one embodiment, single or multiple doses of the affinity purified human polyclonal antibodies may be delivered to a human subject by intravenous administration.

A therapeutically effective amount of the affinity purified human polyclonal antibodies administered to a given individual will, of course, be dependent on a number of factors, including the concentration of the affinity purified human polyclonal antibodies, composition or dosage form, the selected mode of administration, the age and general condition of the individual being treated, the sex of the individual, the severity of the individual's condition, and other factors known to the prescribing physician.

In some embodiments, the affinity purified human polyclonal antibodies of the present invention are administered in a dosage from about 0.1 mg per kg bodyweight to about 10 mg per kg bodyweight, preferably from about 0.3 mg per kg bodyweight to about 3 mg per kg bodyweight, more preferably from about 0.6 mg per kg bodyweight to about 2 mg per kg bodyweight, and most preferably from about 1 mg per kg bodyweight to about 1.5 mg per kg bodyweight. The above mentioned mg per kg dosage refers to the mg of specific antibody against the bacterial antigens, and not necessarily to the total mg of antibody in the preparation which may include antibodies that are not specific to bacterial antigens.

In other embodiments, the affinity purified human polyclonal antibodies are administered with a frequency preferably ranging from approximately once a day to approximately once a month, more preferably from approximately once a week to approximately once every two weeks, most preferably approximately once every two weeks. The dosages for treating chronic infection, e.g., patients with indwelling catheters, post surgical difficult infections and knee replacements, may be different from dosages for treating acute infection, e.g., ICU septic patients. The dosages for prophylactic use may also be different. For prophylactic use, the antibodies may be added to locks on catheters in place of antibiotic locks, or the antibodies may be to peritoneal dialysis solutions, etc. Treating chronic infection, acute infection or prophylactic may use different doses and dosing schedules.

### EXAMPLES

### Example 1

### Bacterial Culture and Antigenic Preparation

Staphylococcus aureus (ATCC #BAA-1556), Streptococcus pyogenes (ATCC #19615) and Escherichia coli 0157 (ATCC #43895) bacterial cells were cultured separately in Bacto™ Tryptic Soy Broth containing 17.0 g/L pancreatic digest of casein; 3.0 g/L enzymatic digest of soybean meal, 5.0 g/L NaCl, 2.5 g/L K₂HPO₄ and 2.5 g/L dextrose (VWR Cat. No. 90000-378; Becton Dickinson Cat. No. 211825; 30% w/v in de-ionized H₂O) at 37° C. on a rotator, e.g., a 2 liter roller bottle that was half filled. Every 12 hours, a 2.5 mL sample was removed from each bacterial culture to determine bacterial counts (OD measurement and serial dilution on blood agar plates) and total protein concentrations (BCA and Lowry protein assays). Bacterial growth was plotted for each culture to determine when the cultures reached saturation. Saturation was typically observed after about 72 hours.

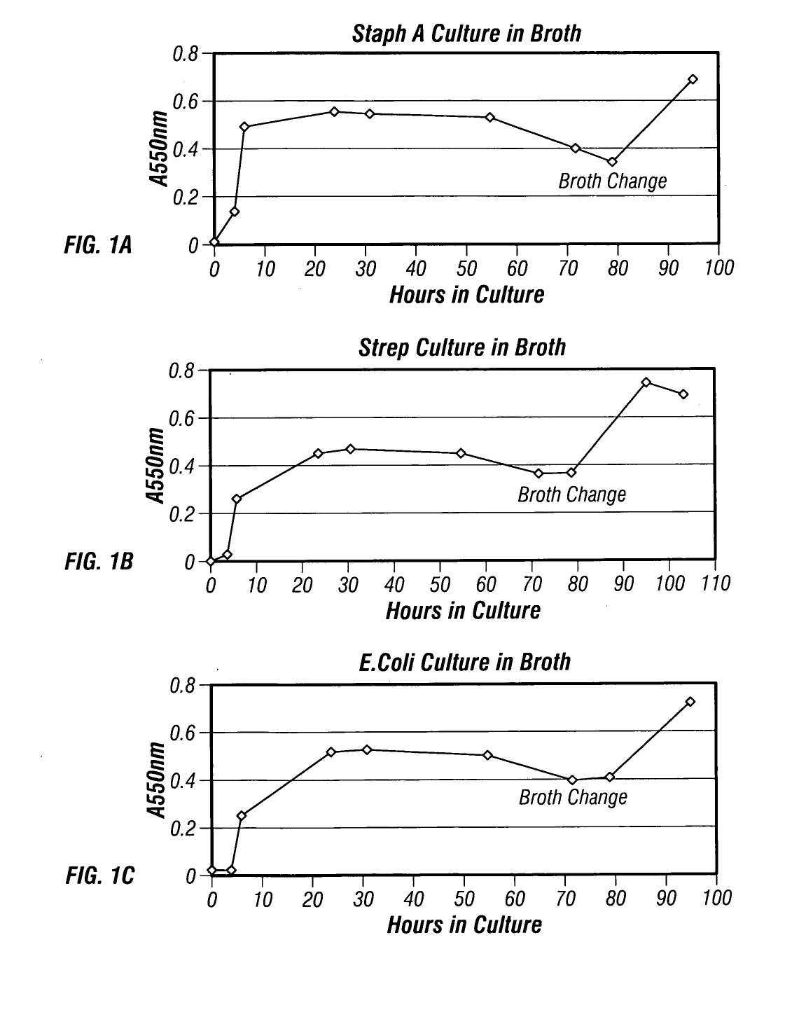

When the cultures reached saturation, the bacteria were washed (involving centrifugation and resuspension) separately three times in a protein-free phosphate-buffered 0.9% NaCl solution (Baxter Cat. No. 2E7125) and precipitated by centrifugation. The pellets were resuspended in 500 mL of the protein-free phosphate-buffered 0.9% NaCl solution (Baxter Cat. No. 2E7125) supplemented with 2 g/L D-(+)-glucose (dextrose) (Sigma Cat. No. G5146), and the bacteria were grown separately for approximately 24 hours until they reached saturation. Bacterial counts and protein concentrations were measured every 12 hours as described above. FIGS. 1A-C show bacterial growth charts for Staphylococcus, Streptococcus and E. coli, respectively.

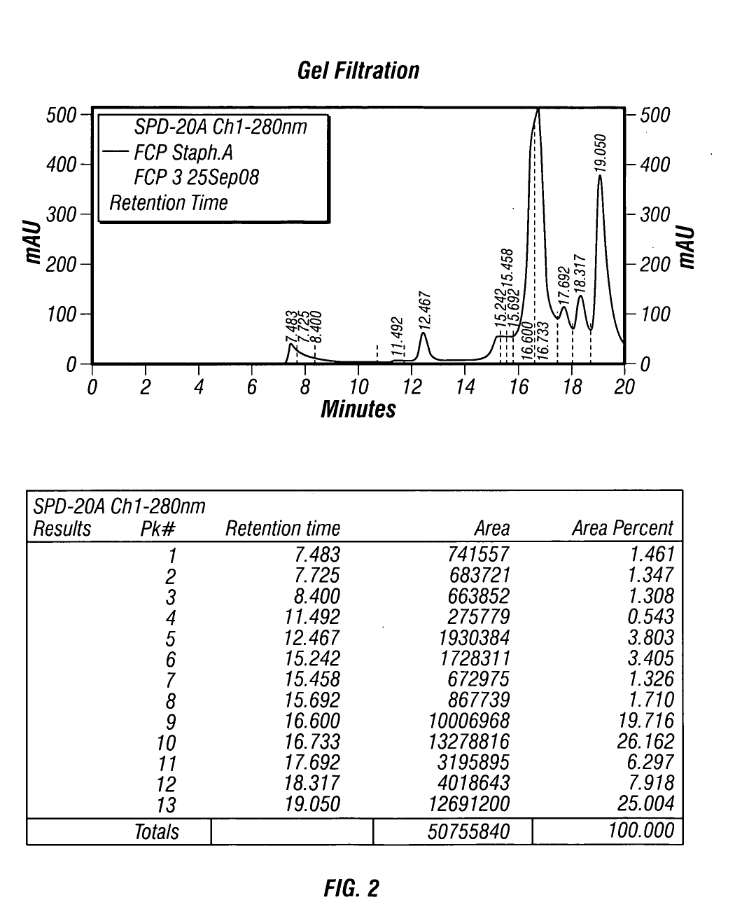

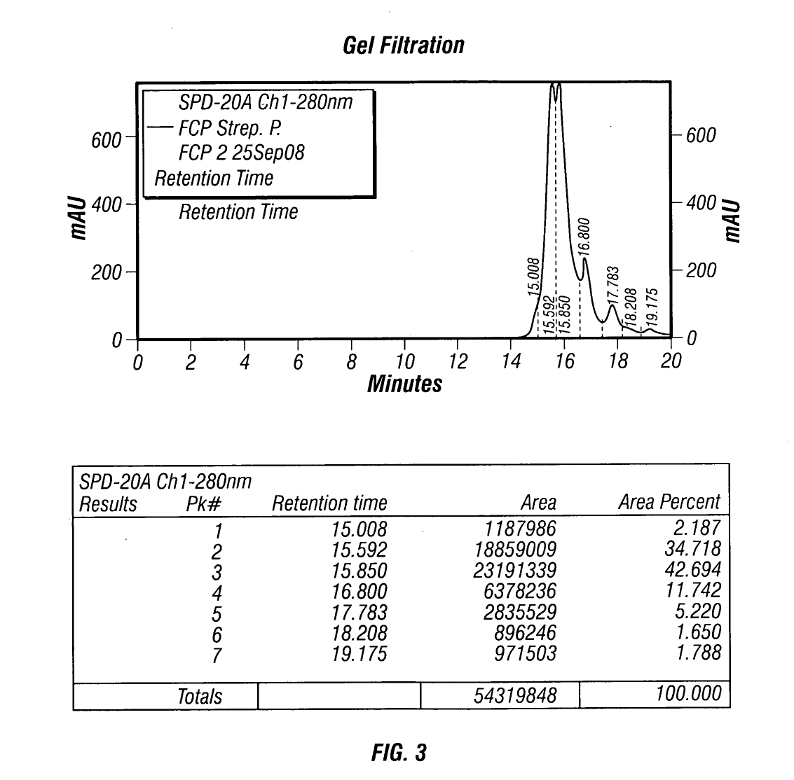

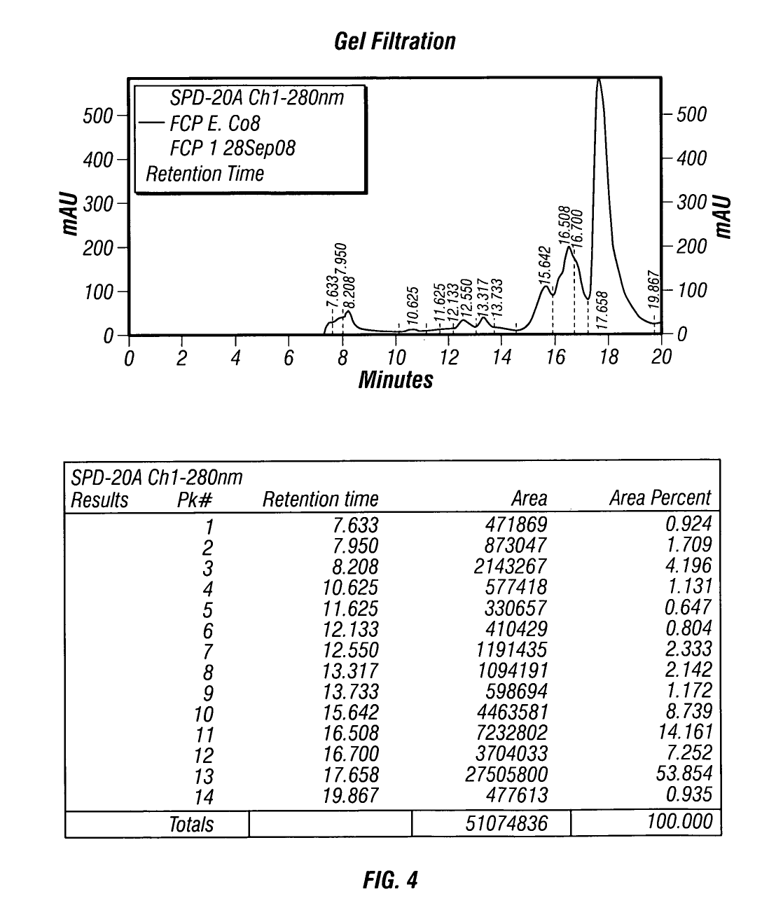

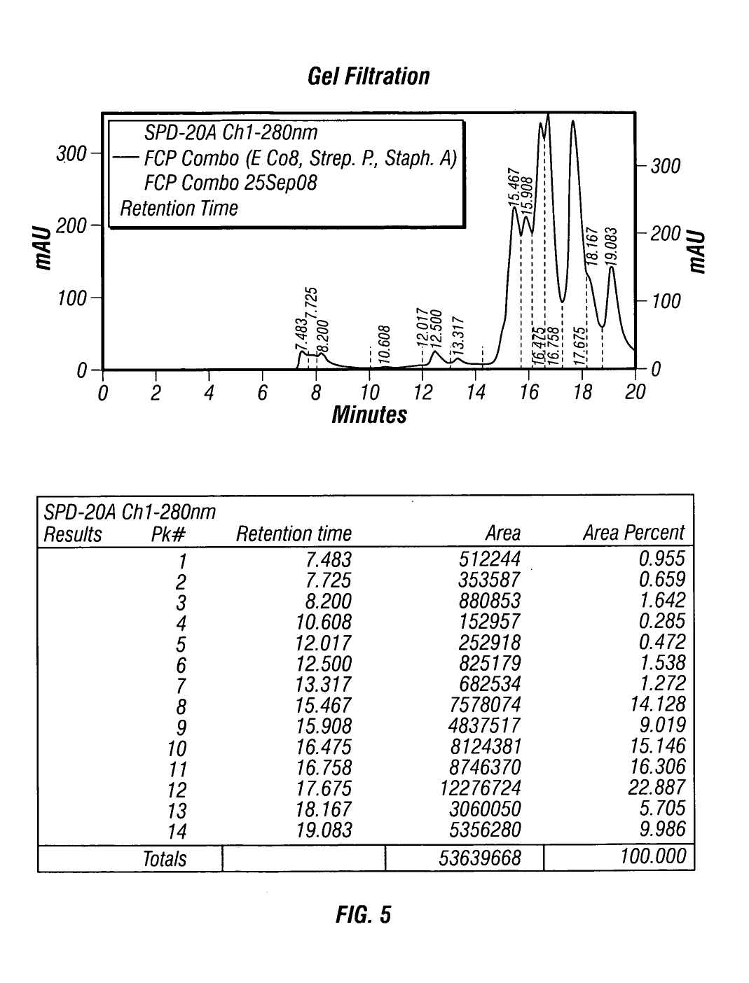

When the cultures reached saturation again, they were freeze-thawed twice, sonicated and homogenized using a Potter-Elvehjem homogenizer to break up the bacterial walls and cell membranes. The homogenates were then precipitated at 3,000 rpm for 30 minutes at 2-8° C. The supernatants were filtered using a 0.2 micron filter to eliminate bacterial contamination. A sample of each supernatant was plated on blood agar plates to determine the presence of live bacteria. Protein concentrations of each supernatant were measured, and the supernatants were combined so as to contain equal amounts of each bacterial antigenic preparation by weight. HPLC gel filtration analysis was carried out for each supernatant and for the combined antigenic preparation. FIGS. 2-5 show HPLC chromatograms for the Staphylococcus, Streptococcus and E. coli antigenic preparations, and for the combined antigenic preparation, respectively.

### Example 2

### Affinity Purification of Human Polyclonal Antibodies

The combined antigenic preparation purified using a 0.2 μM filter is immobilized on sterilized CNBr-activated Sepharose 4B by direct immobilization of the combined antigenic preparation to the sterile, activated gel by overnight incubation at pH 9.0 at 2-8° C. in a rotator. A wash with phosphate buffer removes the uncoupled antigen and any remaining active sites are blocked by glycine. Any suitable substances can be used for the blocking step. In some embodiments, proteins, e.g., serum albumin can be used. Bovine serum albumin can be used. Preferably, human derived proteins, e.g., human serum albumin, are used for the blocking step.

A 25 L volume of lipid-stripped normal human immune plasma is applied to the affinity chromatography column. The immune plasma is charged over the antigen column The antibodies specific to the column bind to the immobilized antigens. The non-specific plasma components are washed off the column by a wash with phosphate buffer. The bound antibodies are eluted at pH 2.5-2.75 and neutralized with phosphate buffer, pH 8.5. The affinity-purified human polyclonal antibodies are then subjected to a solvent/detergent treatment to inactivate enveloped viruses. The inactivation is performed in 1% Triton X-100 and 0.3% Tri-N-butyl phosphate at room temperature for 30 minutes. The solvent/detergent is removed by buffer exchange of the affinity-purified human polyclonal antibodies in an ultra-filtration system against phosphate buffer, pH 5.0-6.0, and concentrated to approximately 9.0 mg/mL.

The concentrated affinity-purified human polyclonal antibodies are further purified using a Planova™ 20 nm filter (Asahi Kasei Medical Cat. Nos. 20N4-000, 20N1-000, 20NZ-300 or 20NZ-120) in order to eliminate any remaining viral particles. Ion exchange chromatography is applied to remove any possible aggregates from the affinity-purified human polyclonal antibodies. Briefly, the affinity purified antibodies are buffer-exchanged into low salt buffer and charged onto an anion or cation exchange column The loosely bound antibodies are removed by an isocratic wash, followed by a linear gradient in the same wash buffer but with elevated salt concentration which separates the monomeric antibodies from the aggregated antibodies and other contaminants. The ion exchange media applied can be Poros HQ from Perseptive BioSystems (Boston, Md.), Capto adhere and Capto S from GE Healthcare (Sweden), or Ceramic Hydroxyapatite (CHT) from Bio-Rad (Hercules, Calif.). Finally, the concentration of the column-processed affinity-purified human polyclonal antibodies is adjusted to about 1.8-2.2 mg/mL, preferably about 2.0 mg/mL, and the product is bottled under sterile conditions at 30 mL per vial so that each vial contains approximately 60 mg of the affinity-purified human polyclonal antibodies.

### Example 3

### Titer Determination of Affinity-Purified Human Polyclonal Antibodies

Human polyclonal antibodies against A. baumannii, P. aeruginosa and S. aureus whole cell extracts were prepared by affinity purification of lipid-stripped normal human immune plasma substantially as described above in Example 2. The concentration of the antibodies was adjusted to 2.0 mg/ml in 10% maltose and 0.03% Polysorbate 80, pH 5.5, and several serial dilutions were prepared in a 2% solution of bovine serum albumin (BSA) in phosphate buffered saline (PBS), pH 7.4, for a titer determination experiment (1:10, 1:100, 1:1,000 and 1:10,000).

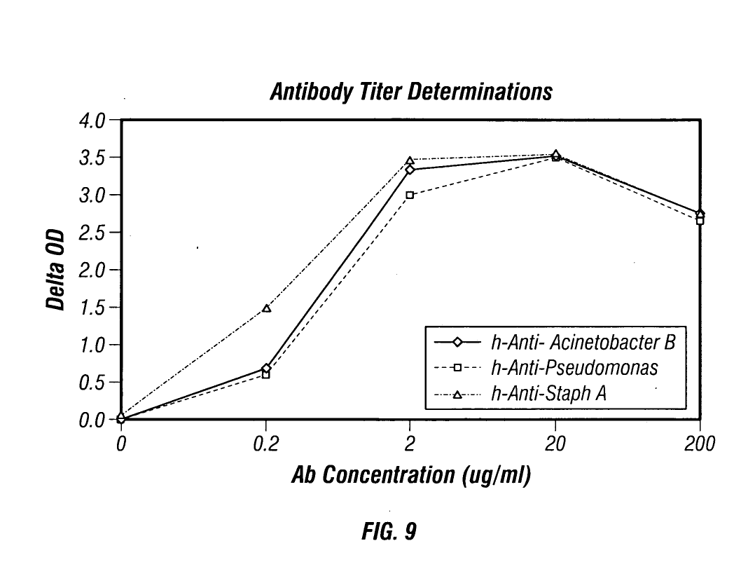

Several 96-well microtiter plates were blocked with 2% BSA in PBS, pH 7.4, and subsequently coated with the A. baumannii, P. aeruginosa and S. aureus antigenic preparations that were used for affinity purification of the human polyclonal antibodies. Each dilution of the antibodies was added to the coated plates, incubated at room temperature for 4 hours and washed with PBS. Anti-human IgG conjugated to horse radish peroxidase (HRP) was then applied to the plates for detection of the captured human antibodies. Color signal was developed using 3,3′,5,5′-tetramethylbenzidine (TMB), and optical density was determined at 450 nm Results of this study are summarized in Table 1 and FIG. 9. Each of the affinity-purified human polyclonal antibodies against A. baumannii, P. aeruginosa and S. aureus was found to have a titer greater than 1:10,000.

TABLE 1
Titer determination of affinity-purified human polyclonal antibodies.

| Antibody | | | | | | | | | | |
|---|---|---|---|---|---|---|---|---|---|---|
| dilution | Antibody | Optical density at 450 nm (OD₄₅₀) | | | | | | | | |
| (from 2 | conc. | α-A. baumannii Ab | | | α-P. aeruginosa Ab | | | α-S. aureus Ab | | |
| mg/ml) | (μg/ml) | Cont. | Test | Delta | Cont. | Test | Delta | Cont. | Test | Delta |
| Blank | 0 | 0.052 | 0.054 | 0.002 | 0.053 | 0.056 | 0.003 | 0.052 | 0.09 | 0.038 |
| 1:10,000 | 0.2 | 0.059 | 0.741 | 0.682 | 0.063 | 0.641 | 0.578 | 0.059 | 1.541 | 1.482 |
| 1:1,000 | 2 | 0.128 | 3.452 | 3.324 | 0.132 | 3.121 | 2.989 | 0.113 | 3.565 | 3.452 |
| 1:100 | 20 | 0.483 | 3.996 | 3.513 | 0.497 | 3.991 | 3.494 | 0.369 | 3.903 | 3.534 |
| 1:10 | 200 | 1.258 | 4.000 | 2.742 | 1.359 | 4.000 | 2.641 | 1.264 | 4.000 | 2.736 |

### Example 4

### Effects of Additional Treatments on the Yield and Titer of Antibodies

Human polyclonal antibodies against S. aureus whole cell extract were prepared by affinity purification of lipid-stripped normal human immune plasma substantially as described above in Example 2, with the sole difference that the antibodies were subjected to additional treatments as described in Table 2. The yields of the antibodies were determined, and the concentration was adjusted to 2.0 mg/ml in 10% maltose and 0.03% Polysorbate 80, pH 5.5. Serial dilutions were then prepared in a 2% solution of bovine serum albumin (BSA) in phosphate buffered saline (PBS), pH 7.4, for a titer determination experiment (1:10,000 and 1,100,000).

Several 96-well microtiter plates were blocked with 2% BSA in PBS, pH 7.4, and subsequently coated with the S. aureus antigenic preparation that was used for affinity purification of the human polyclonal antibodies. Each dilution of the antibodies was added to the coated plates, incubated at room temperature for 4 hours and washed with PBS. Anti-human IgG conjugated to horse radish peroxidase (HRP) was then applied to the plates for detection of the captured human antibodies. Color signal was developed using 3,3′,5,5′-tetramethylbenzidine (TMB), and optical density was determined at 450 nm Results of this study are summarized in Table 2. Each preparation of the affinity-purified human polyclonal antibodies against S. aureus was found to have highly similar yields between 30 and 40 mg/L and titers greater than 1:100,000, regardless of the additional treatments applied.

TABLE 2
Effects of additional treatments on the yield and titer of antibodies.

| | | Optical density at 450 nm (OD₄₅₀) | | | | | |
|---|---|---|---|---|---|---|---|
| Additional | Antibody | S. aureus antibody at | | | S. aureus antibody at | | |
| plasma | yield | 1:10,000 dilution | | | 1:100,000 dilution | | |
| treatment | (mg/ml) | Cont. | Test | Delta | Cont. | Test | Delta |
| Blank | N/A | 0.051 | 0.113 | 0.062 | 0.058 | 0.117 | 0.059 |
| No treatment | 0.033 | 0.058 | 2.626 | 2.568 | 0.049 | 0.503 | 0.454 |
| 0.3% Caprylic acid | 0.037 | 0.058 | 2.267 | 2.209 | 0.053 | 0.525 | 0.472 |
| 7.6% Caprylic acid | 0.039 | 0.070 | 3.884 | 3.814 | 0.052 | 1.051 | 0.999 |
| Ammonium sulfate | 0.036 | 0.063 | 2.147 | 2.084 | 0.052 | 0.434 | 0.382 |
| Caprylic acid and | 0.035 | 0.059 | 2.917 | 2.858 | 0.062 | 0.950 | 0.888 |
| Ammonium sulfate | | | | | | | |
| (CAAS) | | | | | | | |

### Example 5

### Protective Effect of Anti-Staphylococcus Human Polyclonal Antibodies

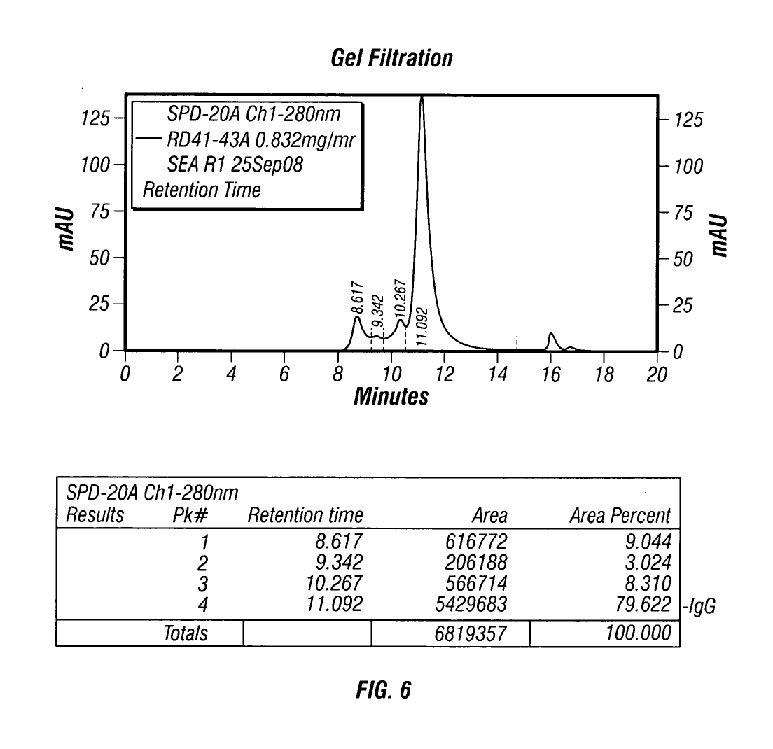

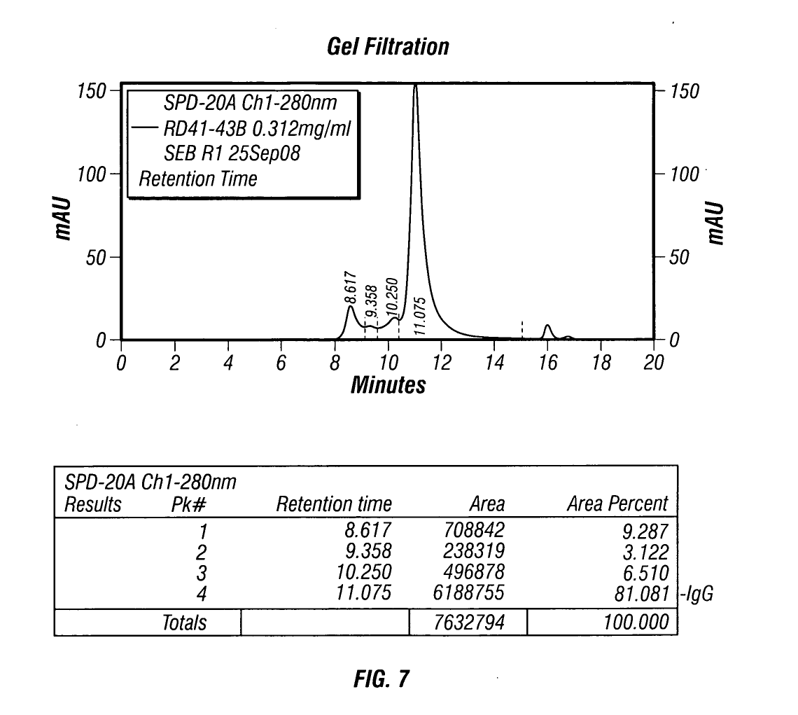

Purified S. aureus enterotoxin A (SEA) and S. aureus enterotoxin B (SEB) were obtained from Sigma-Aldrich, and 3 mg of each toxin was immobilized separately on 5 ml of CNBr-activated Sepharose 4B as described in Example 2. Lipid-stripped human serum was affinity purified as described above. Each cycle of purification yielded 11-30 mg of human polyclonal antibodies specific for SEA and 13-34 mg of human polyclonal antibodies specific for SEB. The resulting affinity-purified human polyclonal antibodies were analyzed by HPLC using the Zorbax GF-250 gel-filtration column FIGS. 6 and 7 demonstrate the chromatographic profiles of the affinity-purified human polyclonal antibodies against SEA and SEB, respectively. The predominant peak in each figure corresponded to immunoglobulin G (IgG), and accounted for approximately 80% of the total protein.

Male BALB/c mice were used to evaluate the protective effect of the affinity-purified human polyclonal antibodies against SEB. It was previously shown in the art that an intraperitoneal administration of 0.1 mg of purified SEB to laboratory mice kills approximately 50% of the animals. Accordingly, 0.1 mg of purified SEB (Sigma) was administered intraperitoneally to male BALB/c mice in a protection, rescue and safety experiments.

Results of this study are summarized in Table 3. Each group included 10 animals. Group 1 was a control group that did not receive any protective antibodies against SEB but received 0.1 mg of purified SEB. This group exhibited 30% mortality after 24 hours. Group 2 was a rescue group that received 0.5 mg of the anti-SEB human polyclonal antibodies 30 minutes after an intraperitoneal administration of 0.1 mg SEB. This group exhibited zero mortality after 24 hours. Group 3 was a protection group that received 0.5 mg of the anti-SEB human polyclonal antibodies 30 minutes before an intraperitoneal administration of 0.1 mg SEB. This group similarly exhibited zero mortality after 24 hours. Finally, Group 4 was a safety group that received 0.5 mg of the anti-SEB human polyclonal antibodies but did not receive any SEB. Much like Groups 2 and 3, Group 4 exhibited zero mortality after 24 hours. The results indicate that treatment with affinity-purified human polyclonal antibodies against SEB before or after an intraperitoneal administration of 0.1 mg SEB reduced 24 hour mortality from 30% to zero, and such treatment is safe.

TABLE 3
Protective effect of human polyclonal
antibodies against SEB in BALB/c mice.

| | Group 1 | Group 2 | Group 3 | Group 4 |
|---|---|---|---|---|
| | (Control) | (Rescue) | (Protection) | (Safety) |
| Number of Animals | 10 | 10 | 10 | 10 |
| SEB (mg) | 0.1 | 0.1 | 0.0 | 0.0 |
| Protective Antibodies | 0.0 | 0.0 | 0.5 | 0.0 |
| to SEB (mg) | | | | |
| Pause (min.) | 30 | 30 | 30 | 30 |
| SEB (mg) | 0.0 | 0.0 | 0.1 | 0.0 |
| Protective Antibodies | 0.0 | 0.5 | 0.0 | 0.5 |
| to SEB (mg) | | | | |
| 24 hour Mortality | 3/10 | 0/10 | 0/10 | 0/10 |

### Example 6

### Protective Effect of Anti-Streptococcus Human Polyclonal Antibodies

Purified Streptococcus Streptolysin 0 toxin (SLO, 9,800 HU/mg) was obtained from Asahi Kasel Pharma Corporation (Japan) and coupled to CNBr-activated Sepharose 4B as described in Example 2. Lipid-stripped human serum was affinity purified as described above.

Male BALB/c mice were used to evaluate the protective effect of the affinity-purified human polyclonal antibodies against SLO. Since published reports show significant variation between lethal doses of SLO in mice, an experiment was conducted to determine SLO's LD₅₀ (a dose at which approximately 50% of the animals die within 24 hours). 36 male BALB/c mice were divided into six equal groups. Each group received an intraperitoneal dose of purified SLO ranging from zero to 10 mg, as shown in Table 4. Mortality of each group was evaluated 24 hours after the injection. Animals in Groups 4-6, which received 0.1 mg or less of the purified SLO, exhibited zero mortality, whereas animals in Groups 1-3, which received 0.5 mg or more of the purified SLO, exhibited 100% mortality. The results indicate that SLO LD₅₀ in BALB/c mice is between 0.1 and 0.5 mg. Accordingly, 0.5 mg of the purified SLO was administered intraperitoneally to male BALB/c mice in a protection and safety experiment similar to the one described in Example 5.

TABLE 4
Lethal dose determination of the Streptococcus
toxin (SLO) in BALB/c mice.

| | | Strep Toxin | |
|---|---|---|---|
| | Number of | (SLO) Dose | 24 Hr Mouse |
| Group | Animals | (mg) | Mortality |
| 1 | 6 | 10.0 | 6/6 |
| 2 | 6 | 1.0 | 6/6 |
| 3 | 6 | 0.5 | 6/6 |
| 4 | 6 | 0.1 | 0/6 |
| 5 | 6 | 0.01 | 0/6 |
| 6 | 6 | 0 | 0/6 |

Results of this study are summarized in Table 5. Each group included 10 animals. Group 1 was a control group that did not receive any protective antibodies against SLO but received 0.5 mg of purified SLO. This group exhibited 100% mortality after 24 hours. Group 2 was a protection group that received 5.0 mg of the anti-SLO human polyclonal antibodies 30 minutes before an intraperitoneal administration of 0.5 mg SLO. This group exhibited zero mortality after 24 hours. Finally, Group 3 was a safety group that received 5.0 mg of the anti-SLO human polyclonal antibodies but did not receive any SLO. Group 3 similarly exhibited zero mortality after 24 hours. The results indicate that treatment with affinity-purified human polyclonal antibodies against SLO before an intraperitoneal administration of 0.5 mg SLO reduced 24 hour mortality from 100% to zero, and such treatment is safe.

TABLE 5
Protective effect of human polyclonal antibodies against
the Streptococcus toxin (SLO) in BALB/c mice.

| | Group 1 | Group 2 | Group 3 |
|---|---|---|---|
| | (Control) | (Protection) | (Safety) |
| Number of Animals | 10 | 10 | 10 |
| Saline (mL) | 1.0 | 0.0 | 0.0 |
| Protective | 0.0 | 5.0 | 5.0 |
| Antibodies (mg) | | | |
| Pause (min.) | 30 | 30 | 30 |
| Streptococcus | 0.5 | 0.5 | 0.0 |
| Toxin (SLO) (mg) | | | |
| Saline (mL) | 0.0 | 0.0 | 1.0 |
| 24 hour Mortality | 10/10 | 0/10 | 0/10 |

### Example 7

### Protective Effect of Anti-E. coli Human Polyclonal Antibodies

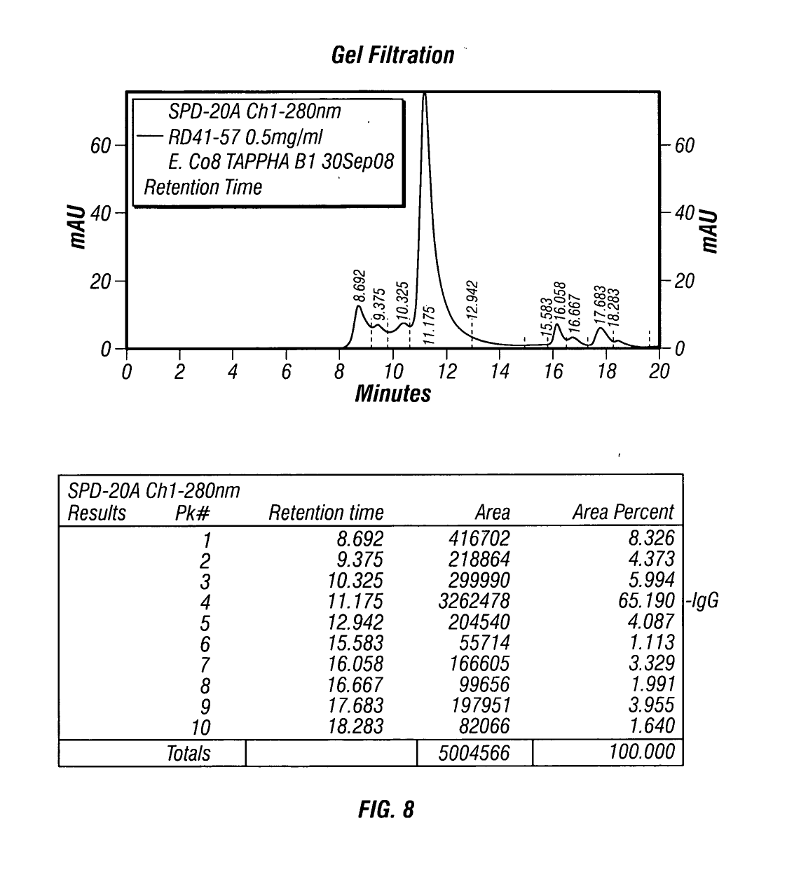

An E. coli antigenic preparation was prepared as described in Example 1 and coupled to CNBr-activated Sepharose 4B as described in Example 2. Lipid-stripped human serum was affinity purified as described above. Approximately 16 mg of affinity-purified human polyclonal antibodies against E. coli was purified from 7 liters of the lipid-stripped human immune serum. The affinity-purified human polyclonal antibodies against E. coli were analyzed by HPLC gel filtration to determine the retention time of the major peak. FIG. 8 shows the chromatographic profile of the anti-E. coli affinity-purified human polyclonal antibodies. The results indicate that the predominant peak approximately corresponds to immunoglobulin G (IgG).

Male Webster Swiss mice were used to evaluate the protective effect of the affinity-purified human polyclonal antibodies against SLO. First, an experiment was conducted to determine the LD₅₀ of the E. coli antigenic preparation. 10 male Swiss mice were split into five equal groups. Each group received an intraperitoneal dose of the E. coli antigenic preparation ranging from zero to 2 mg, as shown in Table 6. Mortality of each group was evaluated 24 hours after the injection. Animals in Group 1, which received no E. coli antigenic preparation, exhibited zero mortality, whereas animals in Groups 3-5, which received 0.5 mg or more of the E. coli antigenic preparation, exhibited 100% mortality. Animals in Group 2, which received 0.1 mg of the E. coli antigenic preparation, exhibited 50% mortality. The results indicate that the LD₅₀ of the E. coli antigenic preparation in Swiss mice is between 0.1 and 0.5 mg. Accordingly, 0.5 mg of the purified E. coli antigenic preparation was administered intraperitoneally to male Swiss mice in a protection and safety study similar to the studies described in Examples 5 and 6.

TABLE 6
Lethal dose determination of the E. coli
antigenic preparation in Swiss mice.

| | Group 1 | Group 2 | Group 3 | Group 4 | Group 5 |
|---|---|---|---|---|---|
| Number of Animals | 2 | 2 | 2 | 2 | 2 |
| Saline (mL) | 2.0 | 0.0 | 0.0 | 0.0 | 0.0 |
| E. coli Antigenic | 0.0 | 0.1 | 0.5 | 1.0 | 2.0 |
| Preparation (mL) | | | | | |
| 24 hour Mortality | 0/2 | 1/2 | 2/2 | 2/2 | 2/2 |

Results of this study are summarized in Table 7. Each group included 6 animals. Group 1 was a control group that did not receive any protective antibodies against E. coli antigens but received 0.5 mg of the purified E. coli antigenic preparation. This group exhibited 100% mortality after 24 hours. Groups 2-4 were protection groups that received between 0.2 mg and 2.0 mg of the anti-E. coli human polyclonal antibodies 60 minutes before an intraperitoneal administration of 0.5 mg of the purified E. coli antigenic preparation. Groups 2 and 3, which received 0.2 mg and 1.0 mg of the anti-E. coli human polyclonal antibodies, exhibited 100% mortality after 24 hours. Group 4, which received 2.0 mg of the anti-E. coli human polyclonal antibodies, exhibited only 50% mortality after 24 hours. Finally, Group 5 was a safety group that received 1.0 mg of the anti-E. coli human polyclonal antibodies but did not receive any E. coli antigenic preparation. Group 5 exhibited zero mortality after 24 hours. The results indicate that treatment with 2.0 mg of the anti-E. coli human polyclonal antibodies before an intraperitoneal administration of 0.5 mg of E. coli antigenic preparation reduced 24 hour mortality from 100% to 50%.

TABLE 7
Protective effect of human polyclonal antibodies
against E. coli antigens in mice.

| | Group 1 | Group 2 | Group 3 | Group 4 | Group 5 |
|---|---|---|---|---|---|
| | (Control) | (Protection) | (Protection) | (Protection) | (Safety) |
| Number of | 6 | 6 | 6 | 6 | 6 |
| Animals | | | | | |
| Saline (mL) | 1.0 | 0.0 | 0.0 | 0.0 | 0.0 |
| Protective | 0.0 | 0.2 | 1.0 | 2.0 | 1.0 |
| Antibodies (mL) | | | | | |
| Pause (min.) | 60 | 60 | 60 | 60 | 60 |
| Antigenic | 0.5 | 0.5 | 0.5 | 0.5 | 0.0 |
| Preparation (mL) | | | | | |
| 24 hour | 6/6 | 6/6 | 6/6 | 3/6 | 0/6 |
| Mortality | | | | | |

The results summarized in Examples 5-7 indicate that affinity-purified human polyclonal antibodies against various bacterial antigens exhibit significant therapeutic and prophylactic properties against these antigens in laboratory animals, and such treatments are safe. Accordingly, it is contemplated that such affinity-purified human polyclonal antibodies will demonstrate similarly significant therapeutic and prophylactic properties in human subjects.

The present invention is further illustrated by the following exemplary embodiments:

1. A method for treating or preventing a bacterial infection, which method comprises administering to a human suffering, suspected of suffering or at risk of suffering from Staphylococcus aureus (S. aureus) infection, a Streptococcus infection, Escherichia coli (E. coli) infection, Pseudomonas aeruginosa (P. aeruginosa) infection, Acinetobacter baumannii (A. baumannii) infection, Enterococcus faecium (E. faecium) infection and/or Clostridium difficile (C. difficile) infection, an effective amount of human polyclonal antibodies affinity purified from a human blood sample with an antigenic preparation comprising cellular and secreted antigens from bacterial cells selected from the group consisting of S. aureus, a Streptococcus, E. coli, P. aeruginosa, A. baumannii, E. faecium, C. difficile and a combination thereof, and optionally, wherein said affinity purified human polyclonal antibodies are purified (e.g., as made more concentrated as compared to the starting or unpurified material) relative to the same human polyclonal antibodies in the unpurified or non-affinity-purified human blood sample, e.g., intravenous immunoglobulin (IVIG) sample, and/or also optionally, the affinity purified human polyclonal antibodies are specific for the bacterial antigen(s) used in the affinity purification, and/or further optionally, said affinity purified human polyclonal antibodies are substantially free of human antibodies that specifically bind to non-bacterial antigens in said human blood sample.

2. The method of embodiment 1, wherein said antigenic preparation comprises cellular and secreted antigens from S. aureus.

3. The method of embodiment 1, wherein said antigenic preparation comprises cellular and secreted antigens from a Streptococcus.

4. The method of embodiment 1, wherein said antigenic preparation comprises cellular and secreted antigens from E. coli.

5. The method of embodiment 1, wherein said antigenic preparation comprises cellular and secreted antigens from P. aeruginosa.

6. The method of embodiment 1, wherein said antigenic preparation comprises cellular and secreted antigens from A. baumannii.

7. The method of embodiment 1, wherein said antigenic preparation comprises cellular and secreted antigens from E. faecium.

8. The method of embodiment 1, wherein said antigenic preparation comprises cellular and secreted antigens from C. difficile.

9. The method of embodiment 1, wherein said antigenic preparation comprises cellular and secreted antigens from any two different bacterial species selected from the group consisting of S. aureus, a Streptococcus, E. coli, P. aeruginosa, A. baumannii, E. faecium and C. difficile.

10. The method of embodiment 1, wherein said antigenic preparation comprises cellular and secreted antigens from any three different bacterial species selected from the group consisting of S. aureus, a Streptococcus, E. coli, P. aeruginosa, A. baumannii, E. faecium and C. difficile.

11. The method of embodiment 1, wherein said antigenic preparation comprises cellular and secreted antigens from any four different bacterial species selected from the group consisting of S. aureus, a Streptococcus, E. coli, P. aeruginosa, A. baumannii, E. faecium and C. difficile.

12. The method of embodiment 1, wherein said antigenic preparation comprises cellular and secreted antigens from any five different bacterial species selected from the group consisting of S. aureus, a Streptococcus, E. coli, P. aeruginosa, A. baumannii, E. faecium and C. difficile.

13. The method of embodiment 1, wherein said antigenic preparation comprises cellular and secreted antigens from any six different bacterial species selected from the group consisting of S. aureus, a Streptococcus, E. coli, P. aeruginosa, A. baumannii, E. faecium and C. difficile.

14. The method of embodiment 1, wherein said antigenic preparation comprises cellular and secreted antigens from each of S. aureus, a Streptococcus, E. coli, P. aeruginosa, A. baumannii, E. faecium and C. difficile.

15. The method of embodiment 1, wherein said antigenic preparation comprises cellular and secreted antigens from each of S. aureus, Streptococcus pyogenes (S. pyogenes), Streptococcus pneumoniae (S. pneumoniae), E. coli, P. aeruginosa, A. baumannii, E. faecium and C. difficile.

16. The method of embodiment 1, wherein the human for treatment is selected from the group consisting of a healthy individual, an infant, a nursing mother, a surgical patient, an individual with a foreign implanted medical device or part, a patient with a fistula, an immunocompromised patient, a patient with a chronic illness, a patient being cared for in a health care facility, a patient with an indwelling catheter, and a patient who has previously suffered from S. aureus infection, a Streptococcus infection, E. coli infection, P. aeruginosa infection, A. baumannii infection, E. faecium infection and/or C. difficile infection.

17. The method of embodiment 16, wherein the implanted medical device or part is selected from the group consisting of a catheter, a prosthesis, an artificial hip, knee or limb, a dialysis access graft, a pacemaker and an implantable defibrillator.

18. The method of embodiment 16, wherein the immunocompromised patient is a chemotherapy patient, a patient receiving a steroid treatment or a patient taking an immunosuppressive drug.

19. The method of embodiment 1, wherein the human suffers, is suspected of suffering or is at risk of suffering from bacteremia.

20. The method of embodiment 1, wherein the S. aureus infection is caused by a S. aureus strain that is resistant to an antibiotic.

21. The method of embodiment 20, wherein the S. aureus infection is caused by a methicillin-resistant strain (MRSA), a vancomycin intermediate strain (VISA) or vancomycin resistant strain (VRSA).

22. The method of embodiment 1, wherein the antigenic preparation comprises S. aureus, Streptococcus, E. coli, P. aeruginosa, A. baumannii, E. faecium and/or C. difficile antigens comprising a peptide, a protein, a polynucleotide, a nucleic acid, a vitamin, a polysaccharide, a carbohydrate, a lipid and/or a complex thereof.

23. The method of embodiment 22, wherein the lipid or the lipid component in the complex is substantially removed in the antigenic preparation.

24. The method of embodiment 1, wherein the polysaccharide, carbohydrate, or the polysaccharide or carbohydrate component in the complex is substantially removed in the antigenic preparation.

25. The method of embodiment 1, wherein S. aureus Protein A is substantially removed in the antigenic preparation.

26. The method of embodiment 1, wherein the antigenic preparation comprises a S. aureus capsular polysaccharide antigen.

27. The method of embodiment 26, wherein the S. aureus capsular polysaccharide antigen is selected from the group consisting of the Type 5 antigen, the Type 8 antigen, and the 336 antigen.

28. The method of embodiment 1, wherein the antigenic preparation comprises a S. aureus toxin.

29. The method of embodiment 28, wherein the S. aureus toxin is selected from the group consisting of a pyrogenic toxin superantigen (PTSAg), an exfoliative toxin and a Staphylococcal toxin.

30. The method of embodiment 29, wherein the pyrogenic toxin superantigen (PTSAg) is the toxic shock syndrome toxin 1 (TSST-1) and/or a S. aureus enterotoxin.

31. The method of embodiment 30, wherein the S. aureus enterotoxin is S. aureus enterotoxin A (SEA) and/or S. aureus enterotoxin B (SEB).

32. The method of embodiment 29, wherein the Staphylococcal toxin is selected from the group consisting of alpha-toxin, beta-toxin, delta-toxin and a bicomponent toxin.

33. The method of embodiment 32, wherein the bicomponent toxin is Panton-Valentine leukocidin (PVL).

34. The method of embodiment 1, wherein the antigenic preparation comprises staphyloxanthin.

35. The method of embodiment 1, wherein the antigenic preparation comprises a S. aureus antigen that confers antibiotic resistance.

36. The method of embodiment 35, wherein the antigen is selected from the group consisting of penicillinase, an altered penicillin-binding protein (PBP2a or PBP2′) encoded by the mecA gene, an aminoglycoside modifying enzyme and an enzyme encoded by the vanA gene.

37. The method of embodiment 1, wherein the antigenic preparation comprises two or more antigens selected from the group consisting of a S. aureus capsular polysaccharide antigen, a S. aureus toxin, staphyloxanthin, and a S. aureus antigen that confers antibiotic resistance.

38. The method of embodiment 1, wherein the antigenic preparation comprises two or more antigens selected from the group consisting of a S. aureus toxin, staphyloxanthin, and a S. aureus antigen that confers antibiotic resistance.

39. The method of embodiment 1, wherein the human suffers, is suspected of suffering or is at risk of suffering from bacterial pneumonia, bacterial meningitis, otitis media, streptococcal pharyngitis (strep throat), scarlet fever, acute rheumatic fever, endocarditis, streptococcal toxic shock syndrome, streptococcal bacteremia or perinatal Group B streptococcal disease.

40. The method of embodiment 1, wherein the Streptococcus infection is caused by Streptococcus pneumoniae (S. pneumoniae), a Group A Streptococcus (GAS) or a Group B Streptococcus (GB S).

41. The method of embodiment 40, wherein the GAS is Streptococcus pyogenes (S. pyogenes).

42. The method of embodiment 40, wherein the GBS is Streptococcus agalactiae (S. agalactiae).

43. The method of embodiment 1, wherein the Streptococcus is selected from the group consisting of Streptococcus pneumoniae (S. pneumoniae), Streptococcus pyogenes (S. pyogenes), Streptococcus agalactiae (S. agalactiae) and a combination thereof.

44. The method of embodiment 40, wherein the Streptococcus infection is caused by a S. pneumoniae strain that is resistant to an antibiotic.

45. The method of embodiment 44, wherein the antibiotic is selected from the group consisting of penicillin, tetracycline, clindamycin, a cephalosporin, a macrolide and a quinolone.

46. The method of embodiment 40, wherein the antigenic preparation comprises two or more S. pneumoniae virulence factors selected from the group consisting of a S. pneumoniae capsular polysaccharide antigen, a S. pneumoniae toxin, autolysin (LytA) and choline binding protein A/pneumococcal surface protein A (CbpA/PspA).

47. The method of embodiment 46, wherein the S. pneumoniae toxin is pneumolysin (Ply).

48. The method of embodiment 41, wherein the antigenic preparation comprises two or more S. pyogenes virulence factors selected from the group consisting of S. pyogenes capsular polysaccharide antigen, a S. pyogenes toxin, M protein, lipoteichoic acid (LTA), a fibronectin-binding protein (protein F), streptokinase, hyaluronidase, streptodornase A-D, C5a peptidase and streptococcal chemokine protease (ScpC).

49. The method of embodiment 48, wherein the S. pyogenes toxin is a streptolysin and/or a streptococcal pyrogenic exotoxin (Spe).

50. The method of embodiment 49, wherein the streptolysin is streptolysin 0 and/or streptolysin S.

51. The method of embodiment 49, wherein the Spe is selected from SpeA, SpeB and/or SpeC.

52. The method of embodiment 42, wherein the antigenic preparation comprises two or more S. agalactiae virulence factors selected from the group consisting of a S. agalactiae capsular polysaccharide antigen, a S. agalactiae toxin, hyaluronidase, C5a peptidase, alpha C protein and glyceraldehyde 3-phosphate dehydrogenase (GAPDH).

53. The method of embodiment 52, wherein the S. agalactiae toxin is (3-hemolysin (cytolysin) and/or CAMP factor (protein B).

54. The method of embodiment 1, wherein the human suffers, is suspected of suffering or is at risk of suffering from gastroenteritis, a urinary tract infection, neonatal meningitis, hemolytic-uremic syndrome (HUS), peritonitis, mastitis, septicemia or Gram-negative pneumonia.

55. The method of embodiment 1, wherein the E. coli infection is caused by E. coli selected from the group consisting of enterotoxigenic E. coli (ETEC), enteropathogenic E. coli (EPEC), enteroinvasive E. coli (EIEC), enterohemorrhagic E. coli (EHEC), enteroaggregative E. coli (EAggEC) and uropathogenic E. coli (UPEC).

56. The method of embodiment 55, wherein the EHEC is a Shiga toxin-producing E. coli (STEC).

57. The method of embodiment 56, wherein the STEC is strain O157:H7.

58. The method of embodiment 55, wherein the antigenic composition comprises two or more E. coli virulence factors selected from the group consisting of an E. coli capsular polysaccharide antigen, K antigen, an enterotoxin, an adhesin, a hemolysin and a Shiga toxin.

59. The method of embodiment 58, wherein the enterotoxin is heat-labile LT enterotoxin and/or heat-stable ST enterotoxin.

60. The method of embodiment 58, wherein the adhesin is a fimbrial adhesin and/or intimin.

61. The method of embodiment 58, wherein the hemolysin is alpha-hemolysin and/or beta-hemolysin.

62. The method of embodiment 1, wherein the E. coli infection is caused by E. coli that is resistant to an antibiotic.

63. The method of embodiment 62, wherein the antibiotic is selected from the group consisting of penicillin, streptomycin, chloramphenicol, ampicillin, cephalosporin and tetracycline.

64. The method of embodiment 1, wherein the antigenic preparation comprises a whole cell extract and a secreted antigen of S. aureus, Streptococcus, E. coli, P. aeruginosa, A. baumannii, E. faecium and/or C. difficile.

65. The method of embodiment 64, wherein the S. aureus antigenic preparation is prepared by the following steps:

a) growing S. aureus cells in a first protein containing S. aureus culture medium for a first period of time;

b) collecting and resuspending the S. aureus cells in a second non-protein containing S. aureus culture medium;

c) growing the S. aureus cells in said second non-protein containing S. aureus culture medium for a second period of time;

d) disrupting the S. aureus cells and collecting a whole cell extract from disrupted S. aureus cells; and

e) collecting a secreted antigen from said second non-protein containing S. aureus culture medium in which the S. aureus cells have grown for said second period of time.

66. The method of embodiment 65, wherein the first protein containing S. aureus culture medium comprises a pancreatic digest of casein, an enzymatic digest of soybean meal, NaCl, K₂HPO₄ and dextrose.

67. The method of embodiment 65, wherein the first period of time is from about 10 hours to about 72 hours.

68. The method of embodiment 65, wherein the second non-protein containing S. aureus culture medium comprises an aqueous solution comprising sodium chloride, sodium phosphate, and optionally comprising a source of carbon.

69. The method of embodiment 65, wherein the second period of time is from about 10 hours to about 48 hours.

70. The method of embodiment 65, wherein the S. aureus cells are disrupted by homogenization, freeze thaw and/or sonication.

71. The method of embodiment 65, wherein the steps d) and e) are performed in one step, the S. aureus cells are disrupted in the second non-protein containing S. aureus culture medium, and insoluble cellular debris are removed to collect whole cell extract and secreted antigens of S. aureus.

72. The method of embodiment 71, wherein the S. aureus cells are disrupted by homogenization, freeze thaw and/or sonication.

73. The method of embodiment 71, wherein the insoluble S. aureus cellular debris are removed by centrifugation or filtration.

74. The method of embodiment 64, wherein the Streptococcus antigenic preparation is prepared by the following steps:

a) growing Streptococcus cells in a first protein containing Streptococcus culture medium for a third period of time;

b) collecting and resuspending the Streptococcus cells in a second non-protein containing Streptococcus culture medium;

c) growing the Streptococcus cells in said second non-protein containing Streptococcus culture medium for a fourth period of time;

d) disrupting the Streptococcus cells and collecting a whole cell extract from disrupted Streptococcus cells; and

e) collecting a secreted antigen from said second non-protein containing Streptococcus culture medium in which the Streptococcus cells have grown for said fourth period of time.

75. The method of embodiment 74, wherein the first protein containing Streptococcus culture medium comprises a pancreatic digest of casein, an enzymatic digest of soybean meal, NaCl, K₂HPO₄ and dextrose.

76. The method of embodiment 74, wherein the third period of time is from about 10 hours to about 72 hours.

77. The method of embodiment 74, wherein the second non-protein containing Streptococcus culture medium comprises an aqueous solution comprising sodium chloride, sodium phosphate, and optionally comprising a source of sugar or carbon.

78. The method of embodiment 74 wherein the fourth period of time is from about 10 hours to about 48 hours.

79. The method of embodiment 74, wherein the Streptococcus cells are disrupted by homogenization, freeze thaw and/or sonication.

80. The method of embodiment 74, wherein the steps d) and e) are performed in one step, the Streptococcus cells are disrupted in the second non-protein containing Streptococcus culture medium, and insoluble cellular debris are removed to collect whole cell extract and secreted antigens of Streptococcus.

81. The method of embodiment 80, wherein the Streptococcus cells are disrupted by homogenization, freeze thaw and/or sonication.

82. The method of embodiment 80, wherein the insoluble Streptococcus cellular debris are removed by centrifugation or filtration.

83. The method of embodiment 64, wherein the E. coli antigenic preparation is prepared by the following steps:

a) growing E. coli cells in a first protein containing E. coli culture medium for a fifth period of time;

b) collecting and resuspending the E. coli cells in a second non-protein containing E. coli culture medium;

c) growing the E. coli cells in said second non-protein containing E. coli culture medium for a sixth period of time;

d) disrupting the E. coli cells and collecting a whole cell extract from disrupted E. coli cells; and

e) collecting a secreted antigen from said second non-protein containing E. coli culture medium in which the E. coli cells have grown for said sixth period of time.

84. The method of embodiment 83, wherein the first protein containing E. coli culture medium comprises a pancreatic digest of casein, an enzymatic digest of soybean meal, NaCl, K₂HPO₄ and dextrose.

85. The method of embodiment 83, wherein the fifth period of time is from about 10 hours to about 72 hours.

86. The method of embodiment 83, wherein the second non-protein containing E. coli culture medium comprises an aqueous solution comprising sodium chloride, sodium phosphate, and optionally comprising a source of carbon.

87. The method of embodiment 83, wherein the sixth period of time is from about 10 hours to about 48 hours.

88. The method of embodiment 83, wherein the E. coli cells are disrupted by homogenization, freeze thaw and/or sonication.

89. The method of embodiment 83, wherein the steps d) and e) are performed in one step, the E. coli cells are disrupted in the second non-protein containing E. coli culture medium, and insoluble cellular debris are removed to collect whole cell extract and secreted antigens of E. coli.

90. The method of embodiment 89, wherein the E. coli cells are disrupted by homogenization, freeze thaw and/or sonication.

91. The method of embodiment 89, wherein the insoluble E. coli cellular debris are removed by centrifugation or filtration.

92. The method of embodiment 1, wherein the secreted antigens of S. aureus comprise S. aureus enterotoxin A (SEA) and/or S. aureus enterotoxin B (SEB).

93. The method of embodiment 92, wherein the SEA has a concentration from about 0.01 μg/ml to about 5 μg/ml.

94. The method of embodiment 92, wherein the SEB has a concentration from about 10 μg/ml to about 400 μg/ml.

95. The method of embodiment 1, wherein the antigenic preparation comprises a S. aureus whole cell extract and S. aureus enterotoxin A (SEA) and/or S. aureus enterotoxin B (SEB).

96. The method of embodiment 95, wherein the SEA has a concentration from about 0.01 μg/ml to about 5 μg/ml.

97. The method of embodiment 95, wherein the SEB has a concentration from about 10 μg/ml to about 400 μg/ml.

98. The method of embodiment 1, wherein the secreted antigens of Streptococcus comprise Streptococcal pyrogenic exotoxin A (SpeA) and/or Streptococcal pyrogenic exotoxin C (SpeC).

99. The method of embodiment 98, wherein the SpeA has a concentration from about 5 μg/ml to about 20 μg/ml.

100. The method of embodiment 98, wherein the SpeC has a concentration from about 0.01 μg/ml to about 10 μg/ml.

101. The method of embodiment 1, wherein the antigenic preparation comprises a Streptococcus whole cell extract and Streptococcal pyrogenic exotoxin A (SpeA) and/or Streptococcal pyrogenic exotoxin C (SpeC).

102. The method of embodiment 101, wherein the SpeA has a concentration from about 5 μg/ml to about 20 μg/ml.

103. The method of embodiment 101, wherein the SpeC has a concentration from about 0.01 μg/ml to about 10 μg/ml.

104. The method of embodiment 1, wherein the secreted antigens of E. coli comprise a Shiga-like toxin.

105. The method of embodiment 104, wherein the Shiga-like toxin has a concentration from about 0.25 μg/ml to about 4 μg/ml.

106. The method of embodiment 1, wherein the antigenic preparation comprises an E. coli whole cell extract and a Shiga-like toxin.

107. The method of embodiment 106, wherein the Shiga-like toxin has a concentration from about 0.25 μg/ml to about 4 μg/ml.

108. The method of embodiment 1, wherein the affinity purified human polyclonal antibodies specific to the bacterial antigen(s) have a concentration ranging from about 10 μg/ml to about 10 mg/ml.

109. The method of embodiment 1, wherein the affinity purified human polyclonal antibodies are purified from about 2 fold to about 50,000 fold relative to the same human polyclonal antibodies in the in the unpurified or non-affinity-purified human blood sample, e.g., intravenous immunoglobulin (IVIG) sample.

110. The method of embodiment 1, wherein the human blood sample is a serum, plasma or whole blood sample.

111. The method of embodiment 1, wherein the human blood sample is collected from a geographic area in which the anti-bacterial treatment is administered, a geographic area in which a recipient of the anti-bacterial treatment resides, or a geographic area to which a recipient of the anti-bacterial treatment intends to travel.

112. The method of embodiment 1, wherein the human blood sample is from a normal human.

113. The method of embodiment 1, wherein the human blood sample is pooled from at least 2 humans.

114. The method of embodiment 1, wherein the human blood sample is pooled from at least 2 normal humans.

115. The method of embodiment 1, further comprising, prior to administering the affinity purified human polyclonal antibodies to the human, conducting an immunotest to determine the presence, absence and/or amount of bacterial antigens in a sample, preferably a blood sample, of the human using the same affinity purified human polyclonal antibodies, to assess the suitability of the human for the therapeutic, removal or preventive treatment, wherein a positive immunotest result indicates that the human is suitable for therapy, removal or prevention of bacterial infection using the affinity purified human polyclonal antibodies.

116. The method of embodiment 115, wherein the immunotest is conducted as a precipitation or an agglutination assay.

117. The method of embodiment 115, wherein the immunotest is conducted in a format selected from the group consisting of an enzyme-linked immunosorbent assay (ELISA), immunoblotting, immunoprecipitation, radioimmunoassay (RIA), immunostaining, latex agglutination, indirect hemagglutination assay (IHA), complement fixation, indirect immunofluorescent assay (IFA), nephelometry, flow cytometry assay, plasmon resonance assay, chemiluminescence assay, lateral flow immunoassay, μ-capture assay, inhibition assay and avidity assay.

118. The method of embodiment 115, wherein the immunotest is conducted in a homogeneous or a heterogeneous assay format.

119. The method of embodiment 1, further comprising, before and after administering the affinity purified human polyclonal antibodies to the human, conducting an immunotest to determine the presence, absence and/or amount of bacterial antigens in a sample, preferably a blood sample, of the human using the same affinity purified human polyclonal antibodies, to monitor the efficacy of the therapeutic, removal or preventive treatment, wherein the absence or reduction in the bacterial antigens after administering the affinity purified human polyclonal antibodies to the human relative to the amount of bacterial antigens before the administration indicates efficacy of the therapeutic, removal or preventive treatment.

120. The method of embodiment 119, wherein the immunotest is conducted as a precipitation or an agglutination assay.

121. The method of embodiment 119, wherein the immunotest is conducted in a format selected from the group consisting of an enzyme-linked immunosorbent assay (ELISA), immunoblotting, immunoprecipitation, radioimmunoassay (RIA), immunostaining, latex agglutination, indirect hemagglutination assay (IHA), complement fixation, indirect immunofluorescent assay (IFA), nephelometry, flow cytometry assay, plasmon resonance assay, chemiluminescence assay, lateral flow immunoassay, μ-capture assay, inhibition assay and avidity assay.

122. The method of embodiment 119, wherein the immunotest is conducted in a homogeneous or a heterogeneous assay format.

123. The method of embodiment 1, further comprising, before and after administering the affinity purified human polyclonal antibodies to the human, conducting an immunotest to determine the presence, absence and/or amount of bacterial antigens in a sample, preferably a blood sample, of the human using the same affinity purified human polyclonal antibodies, to determine an optimal therapeutic or preventive dose of the affinity purified human polyclonal antibodies, wherein the optimal therapeutic, removal or preventive dose is determined based on the amount of the bacterial antigens remaining after administering the affinity purified human polyclonal antibodies to the human and the extent of reduction in the bacterial antigens after administering the affinity purified human polyclonal antibodies to the human relative to the amount of bacterial antigens before the administration.

124. The method of embodiment 123, wherein the immunotest is conducted as a precipitation or an agglutination assay.

125. The method of embodiment 123, wherein the immunotest is conducted in a format selected from the group consisting of an enzyme-linked immunosorbent assay (ELISA), immunoblotting, immunoprecipitation, radioimmunoassay (RIA), immunostaining, latex agglutination, indirect hemagglutination assay (IHA), complement fixation, indirect immunofluorescent assay (IFA), nephelometry, flow cytometry assay, plasmon resonance assay, chemiluminescence assay, lateral flow immunoassay, μ-capture assay, inhibition assay and avidity assay.

126. The method of embodiment 123, wherein the immunotest is conducted in a homogeneous or a heterogeneous assay format.

127. The method of embodiment 1, further comprising conducting an immunotest to determine the presence, absence and/or amount of bacterial antigens in a sample, preferably a blood sample, of the human using the same affinity purified human polyclonal antibodies to assess the suitability of the human for the therapeutic, removal or preventive treatment, to monitor the efficacy of the therapeutic, removal or preventive treatment or to determine an optimal therapeutic or preventive dose, wherein the antigenic preparation comprises a whole cell extract and secreted antigens of S. aureus, a Streptococcus, E. coli, P. aeruginosa, A. baumannii, E. faecium and/or C. difficile.

128. The method of embodiment 1, wherein the affinity purified human polyclonal antibodies specific for the bacterial antigens are administered in a dosage from about 0.3 mg/kg bodyweight to about 1 mg/kg bodyweight.

129. The method of embodiment 1, wherein the affinity purified human polyclonal antibodies are administered approximately biweekly.

130. The method of embodiment 1, wherein the affinity purified human polyclonal antibodies are administered via intravenous, intraperitoneal, intracorporeal, intra-articular, intraventricular, intrathecal, intramuscular, subcutaneous, intranasal, intravaginal, topical or oral administration.

131. The method of embodiment 1, further comprising administering a pharmaceutically acceptable carrier or excipient to the human.

132. The method of embodiment 1, further comprising administering an additional therapeutic or preventive agent.

133. The method of embodiment 132, wherein the additional therapeutic or preventive agent is an antibiotic, an antimicrobial agent, a bactericidal agent, a bacteriostatic agent, or an immunostimulatory compound.

134. The method of embodiment 133, wherein the antibiotic is penicillin, a penicillinase-resistant penicillin, a glycopeptide or an aminoglycoside.

135. The method of embodiment 134, wherein the penicillinase-resistant penicillin is selected from the group consisting of methicillin, oxacillin, cloxacillin, dicloxacillin and flucloxacillin.

136. The method of embodiment 134, wherein the glycopeptide is vancomycin.

137. The method of embodiment 134, wherein the aminoglycoside is selected from the group consisting of kanamycin, gentamicin and streptomycin.

138. The method of embodiment 133, wherein the immunostimulatory compound is a beta-glucan or GM-CSF.

139. The method of embodiment 133, wherein the antimicrobial agent is lysostaphin

140. A pharmaceutical composition for treating or preventing a bacterial infection, which composition comprises an effective amount of human polyclonal antibodies affinity purified from a human blood sample with an antigenic preparation comprising cellular and secreted antigens from bacterial cells selected from the group consisting of S. aureus, a Streptococcus, E. coli, P. aeruginosa, A. baumannii, E. faecium, C. difficile and a combination thereof, and optionally, wherein said affinity purified human polyclonal antibodies are purified (e.g., as made more concentrated as compared to the starting or unpurified material) relative to the same human polyclonal antibodies in the unpurified or non-affinity-purified human blood sample, e.g., intravenous immunoglobulin (IVIG) sample, and/or also optionally, wherein said affinity purified human polyclonal antibodies are specific for the bacterial antigen(s) used in the affinity purification, and/or further optionally wherein said affinity purified human polyclonal antibodies are substantially free of human antibodies that specifically bind to non-bacterial antigens in said human blood sample.

141. The pharmaceutical composition of embodiment 140, wherein said antigenic preparation comprises cellular and secreted antigens from S. aureus.

142. The pharmaceutical composition of embodiment 140, wherein said antigenic preparation comprises cellular and secreted antigens from a Streptococcus.

143. The pharmaceutical composition of embodiment 140, wherein said antigenic preparation comprises cellular and secreted antigens from E. coli.

144. The pharmaceutical composition of embodiment 140, wherein said antigenic preparation comprises cellular and secreted antigens from P. aeruginosa.

145. The pharmaceutical composition of embodiment 140, wherein said antigenic preparation comprises cellular and secreted antigens from A. baumannii.

146. The pharmaceutical composition of embodiment 140, wherein said antigenic preparation comprises cellular and secreted antigens from E. faecium.

147. The pharmaceutical composition of embodiment 140, wherein said antigenic preparation comprises cellular and secreted antigens from C. difficile.

148. The pharmaceutical composition of embodiment 140, wherein said antigenic preparation comprises cellular and secreted antigens from any two different bacterial species selected from the group consisting of S. aureus, a Streptococcus, E. coli, P. aeruginosa, A. baumannii, E. faecium and C. difficile.

149. The pharmaceutical composition of embodiment 140, wherein said antigenic preparation comprises cellular and secreted antigens from any three different bacterial species selected from the group consisting of S. aureus, a Streptococcus, E. coli, P. aeruginosa, A. baumannii, E. faecium and C. difficile.

150. The pharmaceutical composition of embodiment 140, wherein said antigenic preparation comprises cellular and secreted antigens from any four different bacterial species selected from the group consisting of S. aureus, a Streptococcus, E. coli, P. aeruginosa, A. baumannii, E. faecium and C. difficile.

151. The pharmaceutical composition of embodiment 140, wherein said antigenic preparation comprises cellular and secreted antigens from any five different bacterial species selected from the group consisting of S. aureus, a Streptococcus, E. coli, P. aeruginosa, A. baumannii, E. faecium and C. difficile.

152. The pharmaceutical composition of embodiment 140, wherein said antigenic preparation comprises cellular and secreted antigens from any six different bacterial species selected from the group consisting of S. aureus, a Streptococcus, E. coli, P. aeruginosa, A. baumannii, E. faecium and C. difficile.

153. The pharmaceutical composition of embodiment 140, wherein said antigenic preparation comprises cellular and secreted antigens from each of S. aureus, a Streptococcus, E. coli, P. aeruginosa, A. baumannii, E. faecium and C. difficile.

154. The pharmaceutical composition of embodiment 140, wherein said antigenic preparation comprises cellular and secreted antigens from each of S. aureus, Streptococcus pyogenes (S. pyogenes), Streptococcus pneumoniae (S. pneumoniae), E. coli, P. aeruginosa, A. baumannii, E. faecium and C. difficile.

155. The pharmaceutical composition of embodiment 140, wherein the antigenic preparation comprises a whole cell extract and secreted antigens of S. aureus, a Streptococcus, E. coli, P. aeruginosa, A. baumannii, E. faecium and C. difficile.

156. The pharmaceutical composition of embodiment 140, which further comprises a pharmaceutically acceptable carrier or excipient.

157. The pharmaceutical composition of embodiment 140, which further comprises an additional therapeutic or preventive agent.

158. A method for treating or preventing a bacterial infection, which method comprises administering to a human suffering, suspected of suffering or at risk of suffering from Staphylococcus aureus (S. aureus) infection, a Streptococcus infection, Escherichia coli (E. coli) infection, Pseudomonas aeruginosa (P. aeruginosa) infection, Acinetobacter baumannii (A. baumannii) infection, Enterococcus faecium (E. faecium) infection and/or Clostridium difficile (C. difficile) infection, an effective amount of the pharmaceutical composition of any of embodiments 140-157, 162 and 218-228.

159. The method of embodiment 1, wherein the human is suffering, suspected of suffering or at risk of suffering from an additional bacterial infection.

160. The method of embodiment 159, wherein the additional bacterial infection is selected from the group consisting of a Bacillus infection, a Campylobacter infection, an Enterococcus infection, a Helibacter infection, a Listeria infection, a Mycobacterium infection, a Salmonella infection, a Shigella infection, and a combination thereof.

161. The method of embodiment 1, further comprising a step of substantially inactivating and/or removing a virus.

162. The method of embodiment 161, wherein the virus to be substantially inactivated and/or removed is a lipid-enveloped or non-enveloped virus.

163. The method of embodiment 161, wherein a lipid-enveloped virus is substantially inactivated and/or removed by a filtration based on the virus size, using a Planova™ filter and/or a solvent/detergent treatment step.

164. The pharmaceutical composition of embodiment 140, wherein a virus is substantially inactivated and/or removed.

165. The method of embodiment 1, wherein the antigenic preparation comprises two or more antigens selected from the group consisting of a P. aeruginosa adhesin, a P. aeruginosa invasin and a P. aeruginosa toxin.

166. The method of embodiment 165, wherein the P. aeruginosa adhesin is a fimbrial adhesin, a capsular polysaccharide antigen or a mucoid exopolysaccharide antigen.

167. The method of embodiment 166, wherein the fimbrial adhesin comprises N-methyl-phenylalanine.

168. The method of embodiment 166, wherein the capsular polysaccharide antigen is glycocalyx.

169. The method of embodiment 166, wherein the mucoid exopolysaccharide antigen is alginate.

170. The method of embodiment 165, wherein the P. aeruginosa invasin is a protease, a cytotoxin, a hemolysin, or a diffusible pigment.

171. The method of embodiment 170, wherein the protease is an elastase or an alkaline protease.

172. The method of embodiment 170, wherein the cytotoxin is leukocidin.

173. The method of embodiment 170, wherein the hemolysin is a phospholipase or a lecithinase.

174. The method of embodiment 170, wherein the diffusible pigment is pyocyanin or pyochelin.

175. The method of embodiment 165, wherein the P. aeruginosa toxin is lipopolysaccharide (LPS) endotoxin or an extracellular toxin.

176. The method of embodiment 175, wherein the extracellular toxin is P. aeruginosa exoenzyme S (PES) or P. aeruginosa exotoxin A (PEA).

177. The method of embodiment 1, wherein the antigenic preparation comprises two or more C. difficile virulence factors selected from an enterotoxin, a cytotoxin and a binary toxin.

178. The method of embodiment 177, wherein the enterotoxin is C. difficile toxin A.

179. The method of embodiment 177, wherein the cytotoxin is C. difficile toxin B.

180. The method of embodiment 64, wherein the P. aeruginosa antigenic preparation is prepared by the following steps:

a) growing P. aeruginosa cells in a first protein containing P. aeruginosa culture medium for a seventh period of time;

b) collecting and resuspending the P. aeruginosa cells in a second non-protein containing P. aeruginosa culture medium;

c) growing the P. aeruginosa cells in said second non-protein containing P. aeruginosa culture medium for an eighth period of time;

d) disrupting the P. aeruginosa cells and collecting a whole cell extract from disrupted P. aeruginosa cells; and

e) collecting a secreted antigen from said second non-protein containing P. aeruginosa culture medium in which the P. aeruginosa cells have grown for said eighth period of time.

181. The method of embodiment 180, wherein the first protein containing P. aeruginosa culture medium comprises a pancreatic digest of casein, an enzymatic digest of soybean meal, NaCl, K₂HPO₄ and dextrose.

182. The method of embodiment 180, wherein the seventh period of time is from about 10 hours to about 72 hours.

183. The method of embodiment 180, wherein the second non-protein containing P. aeruginosa culture medium comprises an aqueous solution comprising sodium chloride, sodium phosphate, and optionally comprising a source of sugar or carbon.

184. The method of embodiment 180, wherein the eighth period of time is from about 10 hours to about 48 hours.

185. The method of embodiment 180, wherein the P. aeruginosa cells are disrupted by homogenization, freeze thaw and/or sonication.

186. The method of embodiment 180, wherein the steps d) and e) are performed in one step, the P. aeruginosa cells are disrupted in the second non-protein containing P. aeruginosa culture medium, and insoluble cellular debris are removed to collect whole cell extract and secreted antigens of P. aeruginosa.

187. The method of embodiment 186, wherein the P. aeruginosa cells are disrupted by homogenization, freeze thaw and/or sonication.

188. The method of embodiment 186, wherein the insoluble P. aeruginosa cellular debris are removed by centrifugation or filtration.

189. The method of embodiment 64, wherein the C. difficile antigenic preparation is prepared by the following steps:

a) growing C. difficile cells in a first protein containing C. difficile culture medium for a ninth period of time;

b) collecting and resuspending the C. difficile cells in a second non-protein containing C. difficile culture medium;

c) growing the C. difficile cells in said second non-protein containing C. difficile culture medium for a tenth period of time;

d) disrupting the C. difficile cells and collecting a whole cell extract from disrupted C. difficile cells; and

e) collecting a secreted antigen from said second non-protein containing C. difficile culture medium in which the C. difficile cells have grown for said tenth period of time.

190. The method of embodiment 189, wherein the first protein containing C. difficile culture medium comprises a pancreatic digest of casein, proteose peptone #3, beef extract, yeast extract, NaCl, soluble starch, dextrose, cysteine HCl and sodium acetate.

191. The method of embodiment 189, wherein the ninth period of time is from about 10 hours to about 72 hours.

192. The method of embodiment 189, wherein the second non-protein containing C. difficile culture medium comprises an aqueous solution comprising sodium chloride, sodium phosphate, and optionally comprising a source of carbon.

193. The method of embodiment 189, wherein the tenth period of time is from about 10 hours to about 48 hours.

194. The method of embodiment 189, wherein the C. difficile cells are disrupted by homogenization, freeze thaw and/or sonication.

195. The method of embodiment 189, wherein the steps d) and e) are performed in one step, the C. difficile cells are disrupted in the second non-protein containing C. difficile culture medium, and insoluble cellular debris are removed to collect whole cell extract and secreted antigens of C. difficile.

196. The method of embodiment 195, wherein the C. difficile cells are disrupted by homogenization, freeze thaw and/or sonication.

197. The method of embodiment 195, wherein the insoluble C. difficile cellular debris are removed by centrifugation or filtration.

198. The method of embodiment 1, wherein the secreted antigens of P. aeruginosa comprise P. aeruginosa exoenzyme S (PES) and/or P. aeruginosa exotoxin A (PEA).

199. The method of embodiment 198, wherein the PES has a concentration from about 0.01 μg/ml to about 400 μg/ml.

200. The method of embodiment 198, wherein the PEA has a concentration from about 0.01 μg/ml to about 400 μg/ml.

201. The method of embodiment 1, wherein the antigenic preparation comprises a P. aeruginosa whole cell extract and P. aeruginosa exoenzyme S (PES) and/or P. aeruginosa exotoxin A (PEA).

202. The method of embodiment 201, wherein the PES has a concentration from about 0.01 μg/ml to about 400 μg/ml.

203. The method of embodiment 201, wherein the PEA has a concentration from about 0.01 μg/ml to about 400 μg/ml.

204. The method of embodiment 1, wherein the secreted antigens of C. difficile comprise C. difficile toxin A (CTA) and/or C. difficile toxin B (CTB).

205. The method of embodiment 204, wherein the CTA has a concentration from about 0.01 μg/ml to about 400 μg/ml.

206. The method of embodiment 204, wherein the CTB has a concentration from about 0.01 μg/ml to about 400 μg/ml.

207. The method of embodiment 1, wherein the antigenic preparation comprises a C. difficile whole cell extract and C. difficile toxin A (CTA) and/or C. difficile toxin B (CTB).

208. The method of embodiment 207, wherein the CTA has a concentration from about 0.01 μg/ml to about 400 μg/ml.

209. The method of embodiment 207, wherein the CTB has a concentration from about 0.01 μg/ml to about 400 μg/ml.

210. A method for treating or preventing a bacterial infection, which method comprises administering to a human suffering, suspected of suffering or at risk of suffering from Staphylococcus aureus (S. aureus) infection, a Streptococcus infection, Escherichia coli (E. coli) infection, Pseudomonas aeruginosa (P. aeruginosa) infection, Acinetobacter baumannii (A. baumannii) infection, Enterococcus faecium (E. faecium) infection and/or Clostridium difficile (C. difficile) infection, an effective amount of human polyclonal antibodies affinity purified from a human blood sample with an antigenic preparation comprising cellular and/or secreted antigens from two or more different bacterial species selected from the group consisting of S. aureus, a Streptococcus, E. coli, P. aeruginosa, A. baumannii, E. faecium and C. difficile, and optionally, wherein said affinity purified human polyclonal antibodies are purified (e.g., as made more concentrated as compared to the starting or unpurified material) relative to the same human polyclonal antibodies in the unpurified or non-affinity-purified human blood sample, e.g., intravenous immunoglobulin (IVIG) sample, and/or also optionally, wherein said affinity purified human polyclonal antibodies are specific for the bacterial antigen(s) used in the affinity purification, and/or further optionally wherein said affinity purified human polyclonal antibodies are substantially free of human antibodies that specifically bind to non-bacterial antigens in said human blood sample.

211. The method of embodiment 210, wherein said antigenic preparation comprises cellular and/or secreted antigens from any two different bacterial species selected from the group consisting of S. aureus, a Streptococcus, E. coli, P. aeruginosa, A. baumannii, E. faecium and C. difficile.

212. The method of embodiment 210, wherein said antigenic preparation comprises cellular and/or secreted antigens from any three different bacterial species selected from the group consisting of S. aureus, a Streptococcus, E. coli, P. aeruginosa, A. baumannii, E. faecium and C. difficile.

213. The method of embodiment 210, wherein said antigenic preparation comprises cellular and/or secreted antigens from any four different bacterial species selected from the group consisting of S. aureus, a Streptococcus, E. coli, P. aeruginosa, A. baumannii, E. faecium and C. difficile.

214. The method of embodiment 210, wherein said antigenic preparation comprises cellular and/or secreted antigens from any five different bacterial species selected from the group consisting of S. aureus, a Streptococcus, E. coli, P. aeruginosa, A. baumannii, E. faecium and C. difficile.

215. The method of embodiment 210, wherein said antigenic preparation comprises cellular and/or secreted antigens from any six different bacterial species selected from the group consisting of S. aureus, a Streptococcus, E. coli, P. aeruginosa, A. baumannii, E. faecium and C. difficile.

216. The method of embodiment 210, wherein said antigenic preparation comprises cellular and/or secreted antigens from each of S. aureus, a Streptococcus, E. coli, P. aeruginosa, A. baumannii, E. faecium and C. difficile.

217. The method of embodiment 210, wherein said antigenic preparation comprises cellular and/or secreted antigens from each of S. aureus, Streptococcus pyogenes (S. pyogenes), Streptococcus pneumoniae (S. pneumoniae), E. coli, P. aeruginosa, A. baumannii, E. faecium and C. difficile.

218. A pharmaceutical composition for treating or preventing a bacterial infection, which composition comprises an effective amount of human polyclonal antibodies affinity purified from a human blood sample with an antigenic preparation comprising cellular and/or secreted antigens from two or more different bacterial species selected from the group consisting of S. aureus, a Streptococcus, E. coli, P. aeruginosa, A. baumannii, E. faecium and C. difficile, and optionally, wherein said affinity purified human polyclonal antibodies are purified (e.g., as made more concentrated as compared to the starting or unpurified material) relative to the same human polyclonal antibodies in the unpurified or non-affinity-purified human blood sample, e.g., intravenous immunoglobulin (IVIG) sample, and/or also optionally, wherein said affinity purified human polyclonal antibodies are specific for the bacterial antigen(s) used in the affinity purification, and/or further optionally wherein said affinity purified human polyclonal antibodies are substantially free of human antibodies that specifically bind to non-bacterial antigens in said human blood sample.

219. The pharmaceutical composition of embodiment 218, wherein said antigenic preparation comprises cellular and/or secreted antigens from any two different bacterial species selected from the group consisting of S. aureus, a Streptococcus, E. coli, P. aeruginosa, A. baumannii, E. faecium and C. difficile.

220. The pharmaceutical composition of embodiment 218, wherein said antigenic preparation comprises cellular and/or secreted antigens from any three different bacterial species selected from the group consisting of S. aureus, a Streptococcus, E. coli, P. aeruginosa, A. baumannii, E. faecium and C. difficile.

221. The pharmaceutical composition of embodiment 218, wherein said antigenic preparation comprises cellular and/or secreted antigens from any four different bacterial species selected from the group consisting of S. aureus, a Streptococcus, E. coli, P. aeruginosa, A. baumannii, E. faecium and C. difficile.

222. The pharmaceutical composition of embodiment 218, wherein said antigenic preparation comprises cellular and/or secreted antigens from any five different bacterial species selected from the group consisting of S. aureus, a Streptococcus, E. coli, P. aeruginosa, A. baumannii, E. faecium and C. difficile.

223. The pharmaceutical composition of embodiment 218, wherein said antigenic preparation comprises cellular and/or secreted antigens from any six different bacterial species selected from the group consisting of S. aureus, a Streptococcus, E. coli, P. aeruginosa, A. baumannii, E. faecium and C. difficile.

224. The pharmaceutical composition of embodiment 218, wherein said antigenic preparation comprises cellular and/or secreted antigens from each of S. aureus, a Streptococcus, E. coli, P. aeruginosa, A. baumannii, E. faecium and C. difficile.

225. The pharmaceutical composition of embodiment 218, wherein said antigenic preparation comprises cellular and/or secreted antigens from each of S. aureus, Streptococcus pyogenes (S. pyogenes), Streptococcus pneumoniae (S. pneumoniae), E. coli, P. aeruginosa, A. baumannii, E. faecium and C. difficile.

226. The pharmaceutical composition of embodiment 218, wherein the antigenic preparation comprises a whole cell extract and/or secreted antigens of S. aureus, a Streptococcus, E. coli, P. aeruginosa, A. baumannii, E. faecium and C. difficile.

227. The pharmaceutical composition of embodiment 218, which further comprises a pharmaceutically acceptable carrier or excipient.

228. The pharmaceutical composition of embodiment 218, which further comprises an additional therapeutic or preventive agent.

229. A method for treating or preventing a bacterial infection, which method comprises administering to a human suffering, suspected of suffering or at risk of suffering from Staphylococcus aureus (S. aureus) infection, a Streptococcus infection, Escherichia coli (E. coli) infection, Pseudomonas aeruginosa (P. aeruginosa) infection, Acinetobacter baumannii (A. baumannii) infection, Enterococcus faecium (E. faecium) infection and/or Clostridium difficile (C. difficile) infection, an effective amount of human polyclonal antibodies affinity purified from a human blood sample with an antigenic preparation comprising two or more secreted antigens from bacterial cells selected from the group consisting of S. aureus, a Streptococcus, E. coli, P. aeruginosa, A. baumannii, E. faecium, C. difficile, and a combination thereof, and optionally, wherein said affinity purified human polyclonal antibodies are purified (e.g., as made more concentrated as compared to the starting or unpurified material) relative to the same human polyclonal antibodies in the unpurified or non-affinity-purified human blood sample, e.g., intravenous immunoglobulin (IVIG) sample, and/or also optionally, wherein said affinity purified human polyclonal antibodies are specific for the bacterial antigen(s) used in the affinity purification, and/or further optionally wherein said affinity purified human polyclonal antibodies are substantially free of human antibodies that specifically bind to non-bacterial antigens in said human blood sample.

230. The method of embodiment 229, wherein said antigenic preparation comprises two or more secreted antigens from S. aureus.

231. The method of embodiment 229, wherein said antigenic preparation comprises two or more secreted antigens from a Streptococcus.

232. The method of embodiment 231, wherein the Streptococcus is selected from S. pyogenes and S. pneumoniae.

233. The method of embodiment 229, wherein said antigenic preparation comprises two or more secreted antigens from E. coli.

234. The method of embodiment 229, wherein said antigenic preparation comprises two or more secreted antigens from P. aeruginosa.

235. The method of embodiment 229, wherein said antigenic preparation comprises two or more secreted antigens from A. baumannii.

236. The method of embodiment 229, wherein said antigenic preparation comprises two or more secreted antigens from E. faecium.

237. The method of embodiment 229, wherein said antigenic preparation comprises two or more secreted antigens from C. difficile.

238. A pharmaceutical composition for treating or preventing a bacterial infection, which composition comprises an effective amount of human polyclonal antibodies affinity purified from a human blood sample with an antigenic preparation comprising two or more secreted antigens from bacterial cells selected from the group consisting of S. aureus, a Streptococcus, E. coli, P. aeruginosa, A. baumannii, E. faecium, C. difficile, and a combination thereof, and optionally, wherein said affinity purified human polyclonal antibodies are purified (e.g., as made more concentrated as compared to the starting or unpurified material) relative to the same human polyclonal antibodies in the unpurified or non-affinity-purified human blood sample, e.g., intravenous immunoglobulin (IVIG) sample, and/or also optionally, wherein said affinity purified human polyclonal antibodies are specific for the bacterial antigen(s) used in the affinity purification, and/or further optionally wherein said affinity purified human polyclonal antibodies are substantially free of human antibodies that specifically bind to non-bacterial antigens in said human blood sample.

239. The pharmaceutical composition of embodiment 238, wherein said antigenic preparation comprises two or more secreted antigens from S. aureus.

240. The pharmaceutical composition of embodiment 238, wherein said antigenic preparation comprises two or more secreted antigens from a Streptococcus.

241. The pharmaceutical composition of embodiment 240, wherein the Streptococcus is selected from S. pyogenes and S. pneumoniae.

242. The pharmaceutical composition of embodiment 238, wherein said antigenic preparation comprises two or more secreted antigens from E. coli.

243. The pharmaceutical composition of embodiment 238, wherein said antigenic preparation comprises two or more secreted antigens from P. aeruginosa.

244. The pharmaceutical composition of embodiment 238, wherein said antigenic preparation comprises two or more secreted antigens from A. baumannii.

245. The pharmaceutical composition of embodiment 238, wherein said antigenic preparation comprises two or more secreted antigens from E. faecium.

246. The pharmaceutical composition of embodiment 238, wherein said antigenic preparation comprises two or more secreted antigens from C. difficile.

247. A method for treating or preventing a bacterial infection, which method comprises administering to a human suffering, suspected of suffering or at risk of suffering from Streptococcus pneumoniae (S. pneumoniae) infection, Escherichia coli (E. coli) infection, Pseudomonas aeruginosa (P. aeruginosa) infection, Acinetobacter baumannii (A. baumannii) infection, Enterococcus faecium (E. faecium) infection and/or Clostridium difficile (C. difficile) infection, an effective amount of human polyclonal antibodies affinity purified from a human blood sample with an antigenic preparation comprising a cellular and/or secreted antigen from bacterial cells selected from the group consisting of S. pneumoniae, E. coli, P. aeruginosa, A. baumannii, E. faecium, C. difficile, and a combination thereof, and optionally, wherein said affinity purified human polyclonal antibodies are purified (e.g., as made more concentrated as compared to the starting or unpurified material) relative to the same human polyclonal antibodies in the unpurified or non-affinity-purified human blood sample, e.g., intravenous immunoglobulin (IVIG) sample, and/or also optionally, wherein said affinity purified human polyclonal antibodies are specific for the bacterial antigen(s) used in the affinity purification, and/or further optionally wherein said affinity purified human polyclonal antibodies are substantially free of human antibodies that specifically bind to non-bacterial antigens in said human blood sample.

248. The method of embodiment 247, wherein said antigenic preparation comprises a cellular and/or secreted antigen from S. pneumoniae.

249. The method of embodiment 247, wherein said antigenic preparation comprises a cellular and/or secreted antigen from E. coli.

250. The method of embodiment 247, wherein said antigenic preparation comprises a cellular and/or secreted antigen from P. aeruginosa.

251. The method of embodiment 247, wherein said antigenic preparation comprises a cellular and/or secreted antigen from A. baumannii.

252. The method of embodiment 247, wherein said antigenic preparation comprises a cellular and/or secreted antigen from E. faecium.

253. The method of embodiment 247, wherein said antigenic preparation comprises a cellular and/or secreted antigen from C. difficile.

254. A pharmaceutical composition for treating or preventing a bacterial infection, which composition comprises an effective amount of human polyclonal antibodies affinity purified from a human blood sample with an antigenic preparation comprising a cellular and/or secreted antigen from bacterial cells selected from the group consisting of S. pneumoniae, E. coli, P. aeruginosa, A. baumannii, E. faecium, C. difficile, and a combination thereof, and optionally, wherein said affinity purified human polyclonal antibodies are purified (e.g., as made more concentrated as compared to the starting or unpurified material) relative to the same human polyclonal antibodies in the unpurified or non-affinity-purified human blood sample, e.g., intravenous immunoglobulin (IVIG) sample, and/or also optionally, wherein said affinity purified human polyclonal antibodies are specific for the bacterial antigen(s) used in the affinity purification, and/or further optionally wherein said affinity purified human polyclonal antibodies are substantially free of human antibodies that specifically bind to non-bacterial antigens in said human blood sample.

255. The pharmaceutical composition of embodiment 254, wherein said antigenic preparation comprises a cellular and/or secreted antigen from S. pneumoniae.

256. The pharmaceutical composition of embodiment 254, wherein said antigenic preparation comprises a cellular and/or secreted antigen from E. coli.

257. The pharmaceutical composition of embodiment 254, wherein said antigenic preparation comprises a cellular and/or secreted antigen from P. aeruginosa.

258. The pharmaceutical composition of embodiment 254, wherein said antigenic preparation comprises a cellular and/or secreted antigen from A. baumannii.

259. The pharmaceutical composition of embodiment 254, wherein said antigenic preparation comprises a cellular and/or secreted antigen from E. faecium.

260. The pharmaceutical composition of embodiment 254, wherein said antigenic preparation comprises a cellular and/or secreted antigen from C. difficile.

The above examples are included for illustrative purposes only and are not intended to limit the scope of the invention. Many variations to those described above are possible. Since modifications and variations to the examples described above will be apparent to those of skill in this art, it is intended that this invention be limited only by the scope of the claims.

Unless indicated otherwise, all publications and documents cited herein are incorporated by reference in their entireties. Citation of publications or documents is not intended as an admission that any of such publications or documents are pertinent prior art, nor does it constitute any admission as to the contents or date of these publications or documents.

1. A pharmaceutical composition for treating or preventing a bacterial infection, which composition comprises an effective amount of human polyclonal antibodies affinity purified from a human blood sample with an antigenic preparation comprising cellular and secreted antigens from bacterial cells selected from the group consisting of Staphylococcus aureus (S. aureus), a Streptococcus, Escherichia coli (E. coli), Pseudomonas aeruginosa (P. aeruginosa), Clostridium difficile (C. difficile) and a combination thereof, and optionally, wherein said affinity purified human polyclonal antibodies are purified relative to the same human polyclonal antibodies in the unpurified or non-affinity-purified human blood sample, and/or also optionally, wherein said affinity purified human polyclonal antibodies are specific for the bacterial antigens used in the affinity purification, and/or further optionally wherein said affinity purified human polyclonal antibodies are substantially free of human antibodies that specifically bind to non-bacterial antigens in said human blood sample.

2. The pharmaceutical composition of claim 1, wherein the affinity purified human polyclonal antibodies specific to the bacterial antigens have a concentration ranging from about 10 μg/ml to about 10 mg/ml.

3. The pharmaceutical composition of claim 1, wherein the affinity purified human polyclonal antibodies are purified from about 2 fold to about 50,000 fold relative to the same human polyclonal antibodies in the in the unpurified or non-affinity-purified human blood sample.

4. The pharmaceutical composition of claim 1, wherein the human blood sample is from a normal human.

5. The pharmaceutical composition of claim 1, wherein the human blood sample is pooled from at least 2 humans.

6. The pharmaceutical composition of claim 1, wherein said antigenic preparation comprises cellular and secreted antigens from:
a) any two different bacterial species selected from the group consisting of S. aureus, a Streptococcus, E. coli, P. aeruginosa and C. difficile; or
b) any three different bacterial species selected from the group consisting of S. aureus, a Streptococcus, E. coli, P. aeruginosa and C. difficile; or
c) any four different bacterial species selected from the group consisting of S. aureus, a Streptococcus, E. coli, P. aeruginosa and C. difficile; or
d) each of S. aureus, a Streptococcus, E. coli, P. aeruginosa and C. difficile; or
e) each of S. aureus, Streptococcus pyogenes (S. pyogenes), Streptococcus pneumoniae (S. pneumoniae), E. coli, P. aeruginosa and C. difficile.

7. The pharmaceutical composition of claim 1, wherein the antigenic preparation comprises two or more antigens selected from the group consisting of a S. aureus capsular polysaccharide antigen, a S. aureus toxin, staphyloxanthin, and a S. aureus antigen that confers antibiotic resistance.

8. The pharmaceutical composition of claim 1, wherein the antigenic preparation comprises a S. aureus capsular polysaccharide antigen.

9. The pharmaceutical composition of claim 1, wherein the antigenic preparation comprises a S. aureus toxin.

10. The pharmaceutical composition of claim 1, wherein the antigenic preparation comprises a whole cell extract and a secreted antigen of S. aureus, a Streptococcus, E. coli, P. aeruginosa and/or C. difficile.

11. The pharmaceutical composition of claim 10, wherein the antigenic preparation comprises a S. aureus whole cell extract and S. aureus enterotoxin A (SEA) and/or S. aureus enterotoxin B (SEB).

12. The pharmaceutical composition of claim 10, wherein the antigenic preparation comprises a Streptococcus whole cell extract and Streptococcal pyrogenic exotoxin A (SpeA) and/or Streptococcal pyrogenic exotoxin C (SpeC).

13. The pharmaceutical composition of claim 10, wherein the antigenic preparation comprises an E. coli whole cell extract and a Shiga-like toxin.

14. The pharmaceutical composition of claim 10, wherein the antigenic preparation is prepared by the following steps:
a) growing bacterial cells in a first protein containing culture medium;
b) collecting and resuspending the bacterial cells in a second non-protein containing culture medium;
c) growing the bacterial cells in the second non-protein containing culture medium;
d) disrupting the bacterial cells and collecting a whole cell extract from the disrupted bacterial cells; and
e) collecting a secreted antigen from said second non-protein containing culture medium in which the bacterial cells have grown.

15. A method for treating or preventing a bacterial infection, which method comprises administering to a human suffering, suspected of suffering or at risk of suffering from S. aureus infection, a Streptococcus infection, E. coli infection, P. aeruginosa infection and/or C. difficile infection, an effective amount of the pharmaceutical composition of claim 1.

16. The method of claim 15, wherein said antigenic preparation comprises cellular and secreted antigens from:
a) any two different bacterial species selected from the group consisting of S. aureus, a Streptococcus, E. coli, P. aeruginosa and C. difficile; or
b) any three different bacterial species selected from the group consisting of S. aureus, a Streptococcus, E. coli, P. aeruginosa and C. difficile; or
c) any four different bacterial species selected from the group consisting of S. aureus, a Streptococcus, E. coli, P. aeruginosa and C. difficile; or
d) each of S. aureus, a Streptococcus, E. coli, P. aeruginosa and C. difficile; or
e) each of S. aureus, Streptococcus pyogenes (S. pyogenes), Streptococcus pneumoniae (S. pneumoniae), E. coli, P. aeruginosa and C. difficile.

17. The method of claim 15, wherein the human for treatment is selected from the group consisting of a healthy individual, an infant, a nursing mother, a surgical patient, an individual with a foreign implanted medical device or part, a patient with a fistula, an immunocompromised patient, a patient with a chronic illness, a patient being cared for in a health care facility, a patient with an indwelling catheter, and a patient who has previously suffered from S. aureus infection, a Streptococcus infection, E. coli infection, P. aeruginosa infection and/or C. difficile infection.

18. The method of claim 15, wherein the human suffers, is suspected of suffering or is at risk of suffering from bacteremia.

19. The method of claim 15, wherein the S. aureus infection is caused by a S. aureus strain that is resistant to an antibiotic.

20. The method of claim 19, wherein the S. aureus infection is caused by a methicillin-resistant strain (MRSA), a vancomycin intermediate strain (VISA) or vancomycin resistant strain (VRSA).

21. The method of claim 15, wherein the human suffers, is suspected of suffering or is at risk of suffering from bacterial pneumonia, bacterial meningitis, otitis media, streptococcal pharyngitis (strep throat), scarlet fever, acute rheumatic fever, endocarditis, streptococcal toxic shock syndrome, streptococcal bacteremia or perinatal Group B streptococcal disease.

22. The method of claim 15, wherein the Streptococcus infection is caused by Streptococcus pneumoniae (S. pneumoniae), a Group A Streptococcus (GAS) or a Group B Streptococcus (GBS).

23. The method of claim 15, wherein the Streptococcus is selected from the group consisting of Streptococcus pneumoniae (S. pneumoniae), Streptococcus pyogenes (S. pyogenes), Streptococcus agalactiae (S. agalactiae) and a combination thereof.

24. The method of claim 15, wherein the human suffers, is suspected of suffering or is at risk of suffering from gastroenteritis, a urinary tract infection, neonatal meningitis, hemolytic-uremic syndrome (HUS), peritonitis, mastitis, septicemia or Gram-negative pneumonia.

25. The method of claim 15, wherein the E. coli infection is caused by E. coli selected from the group consisting of enterotoxigenic E. coli (ETEC), enteropathogenic E. coli (EPEC), enteroinvasive E. coli (EIEC), enterohemorrhagic E. coli (EHEC), enteroaggregative E. coli (EAggEC) and uropathogenic E. coli (UPEC).

26. The method of claim 15, further comprising, prior to administering the affinity purified human polyclonal antibodies to the human, conducting an immunotest to determine the presence, absence and/or amount of bacterial antigens in a blood sample of the human using the same affinity purified human polyclonal antibodies, to assess the suitability of the human for the therapeutic, removal or preventive treatment, wherein a positive immunotest result indicates that the human is suitable for therapy, removal or prevention of bacterial infection using the affinity purified human polyclonal antibodies.

27. The method of claim 15, further comprising, before and after administering the affinity purified human polyclonal antibodies to the human, conducting an immunotest to determine the presence, absence and/or amount of bacterial antigens in a blood sample of the human using the same affinity purified human polyclonal antibodies, to monitor the efficacy of the therapeutic, removal or preventive treatment, wherein the absence or reduction in the bacterial antigens after administering the affinity purified human polyclonal antibodies to the human relative to the amount of bacterial antigens before the administration indicates efficacy of the therapeutic, removal or preventive treatment.

28. The method of claim 15, further comprising, before and after administering the affinity purified human polyclonal antibodies to the human, conducting an immunotest to determine the presence, absence and/or amount of bacterial antigens in a blood sample of the human using the same affinity purified human polyclonal antibodies, to determine an optimal therapeutic or preventive dose of the affinity purified human polyclonal antibodies, wherein the optimal therapeutic, removal or preventive dose is determined based on the amount of the bacterial antigens remaining after administering the affinity purified human polyclonal antibodies to the human and the extent of reduction in the bacterial antigens after administering the affinity purified human polyclonal antibodies to the human relative to the amount of bacterial antigens before the administration.

29. The method of claim 15, further comprising conducting an immunotest to determine the presence, absence and/or amount of bacterial antigens in a blood sample of the human using the same affinity purified human polyclonal antibodies to assess the suitability of the human for the therapeutic, removal or preventive treatment, to monitor the efficacy of the therapeutic, removal or preventive treatment or to determine an optimal therapeutic or preventive dose, wherein the antigenic preparation comprises a whole cell extract and a secreted antigen of S. aureus, a Streptococcus, E. coli, P. aeruginosa and/or C. difficile.

30. A pharmaceutical composition for treating or preventing a bacterial infection, which composition comprises an effective amount of human polyclonal antibodies affinity purified from a human blood sample with an antigenic preparation comprising cellular and/or secreted antigens from two or more different bacterial species selected from the group consisting of Staphylococcus aureus (S. aureus), a Streptococcus, Escherichia coli (E. coli), Pseudomonas aeruginosa (P. aeruginosa) and Clostridium difficile (C. difficile), and optionally, wherein said affinity purified human polyclonal antibodies are purified relative to the same human polyclonal antibodies in the unpurified or non-affinity-purified human blood sample, and/or also optionally, wherein said affinity purified human polyclonal antibodies are specific for the bacterial antigens used in the affinity purification, and/or further optionally wherein said affinity purified human polyclonal antibodies are substantially free of human antibodies that specifically bind to non-bacterial antigens in said human blood sample.

31. A method for treating or preventing a bacterial infection, which method comprises administering to a human suffering, suspected of suffering or at risk of suffering from S. aureus infection, a Streptococcus infection, E. coli infection, P. aeruginosa infection and/or C. difficile infection, an effective amount of the pharmaceutical composition of claim 30.

32. A pharmaceutical composition for treating or preventing a bacterial infection, which composition comprises an effective amount of human polyclonal antibodies affinity purified from a human blood sample with an antigenic preparation comprising two or more secreted antigens from bacterial cells selected from the group consisting of Staphylococcus aureus (S. aureus), a Streptococcus, Escherichia coli (E. coli), Pseudomonas aeruginosa (P. aeruginosa), Clostridium difficile (C. difficile) and a combination thereof, and optionally, wherein said affinity purified human polyclonal antibodies are purified relative to the same human polyclonal antibodies in the unpurified or non-affinity-purified human blood sample, and/or also optionally, wherein said affinity purified human polyclonal antibodies are specific for the bacterial antigens used in the affinity purification, and/or further optionally wherein said affinity purified human polyclonal antibodies are substantially free of human antibodies that specifically bind to non-bacterial antigens in said human blood sample.

33. A method for treating or preventing a bacterial infection, which method comprises administering to a human suffering, suspected of suffering or at risk of suffering from S. aureus infection, a Streptococcus infection, E. coli infection, P. aeruginosa infection and/or C. difficile infection, an effective amount of the pharmaceutical composition of claim 32.

34. A pharmaceutical composition for treating or preventing a bacterial infection, which composition comprises an effective amount of human polyclonal antibodies affinity purified from a human blood sample with an antigenic preparation comprising a cellular and/or secreted antigen from bacterial cells selected from the group consisting of Streptococcus pneumoniae (S. pneumoniae), Escherichia coli (E. coli), Pseudomonas aeruginosa (P. aeruginosa), Clostridium difficile (C. difficile) and a combination thereof, and optionally, wherein said affinity purified human polyclonal antibodies are purified relative to the same human polyclonal antibodies in the unpurified or non-affinity-purified human blood sample, and/or also optionally, wherein said affinity purified human polyclonal antibodies are specific for the bacterial antigens used in the affinity purification, and/or further optionally wherein said affinity purified human polyclonal antibodies are substantially free of human antibodies that specifically bind to non-bacterial antigens in said human blood sample.

35. A method for treating or preventing a bacterial infection, which method comprises administering to a human suffering, suspected of suffering or at risk of suffering from S. pneumoniae infection, E. coli infection, P. aeruginosa infection and/or C. difficile infection, an effective amount of the pharmaceutical composition of claim 34.

36. A pharmaceutical composition for treating or preventing a bacterial infection, which composition comprises an effective amount of human polyclonal antibodies affinity purified from a human blood sample with an antigenic preparation comprising cellular and secreted antigens from bacterial cells selected from the group consisting of Staphylococcus aureus (S. aureus), a Streptococcus, Escherichia coli (E. coli), Pseudomonas aeruginosa (P. aeruginosa), Acinetobacter baumannii (A. baumannii), Enterococcus faecium (E. faecium), Clostridium difficile (C. difficile) and a combination thereof, and optionally, wherein said affinity purified human polyclonal antibodies are purified relative to the same human polyclonal antibodies in the unpurified or non-affinity-purified human blood sample, and/or also optionally, wherein said affinity purified human polyclonal antibodies are specific for the bacterial antigens used in the affinity purification, and/or further optionally wherein said affinity purified human polyclonal antibodies are substantially free of human antibodies that specifically bind to non-bacterial antigens in said human blood sample.

37. The pharmaceutical composition of claim 36, wherein said antigenic preparation comprises cellular and secreted antigens from:
a) any two different bacterial species selected from the group consisting of S. aureus, a Streptococcus, E. coli, P. aeruginosa, A. baumannii, E. faecium and C. difficile; or
b) any three different bacterial species selected from the group consisting of S. aureus, a Streptococcus, E. coli, P. aeruginosa, A. baumannii, E. faecium and C. difficile; or
c) any four different bacterial species selected from the group consisting of S. aureus, a Streptococcus, E. coli, P. aeruginosa, A. baumannii, E. faecium and C. difficile; or
d) any five different bacterial species selected from the group consisting of S. aureus, a Streptococcus, E. coli, P. aeruginosa, A. baumannii, E. faecium and C. difficile; or
e) any six different bacterial species selected from the group consisting of S. aureus, a Streptococcus, E. coli, P. aeruginosa, A. baumannii, E. faecium and C. difficile; or
f) each of S. aureus, a Streptococcus, E. coli, P. aeruginosa, A. baumannii, E. faecium and C. difficile; or
g) each of S. aureus, Streptococcus pyogenes (S. pyogenes), Streptococcus pneumoniae (S. pneumoniae), E. coli, P. aeruginosa, A. baumannii, E. faecium and C. difficile.

38. The pharmaceutical composition of claim 36, wherein the antigenic preparation comprises a whole cell extract and a secreted antigen of S. aureus, a Streptococcus, E. coli, P. aeruginosa, A. baumannii, E. faecium and/or C. difficile.

39. The pharmaceutical composition of claim 38, wherein the antigenic preparation is prepared by the following steps:
a) growing bacterial cells in a first protein containing culture medium;
b) collecting and resuspending the bacterial cells in a second non-protein containing culture medium;
c) growing the bacterial cells in the second non-protein containing culture medium;
d) disrupting the bacterial cells and collecting a whole cell extract from the disrupted bacterial cells; and
e) collecting a secreted antigen from said second non-protein containing culture medium in which the bacterial cells have grown.

40. A method for treating or preventing a bacterial infection, which method comprises administering to a human suffering, suspected of suffering or at risk of suffering from S. aureus infection, a Streptococcus infection, E. coli infection, P. aeruginosa infection, A. baumannii infection, E. faecium infection and/or C. difficile infection, an effective amount of the pharmaceutical composition of claim 36.
